(19) United States
(12) Patent Application Publication
(10) Pub. No.: US 2007/0281312 A1
(43) Pub. Date: Dec. 6, 2007

(54) NUCLEIC ACID-BINDING CHIPS FOR THE DETECTION OF PHOSPHATE DEFICIENCY CONDITIONS IN THE FRAMEWORK OF BIOPROCESS MONITORING

(76) Inventors: Thomas SCHWEDER, Greifswald (DE); Britta JURGEN, Greifswald (DE); Stefan EVERS, Mettmann (DE); Karl-Heinz MAURER, Erkrath (DE); Le Thi HOI, Greifswald (DE); Michael HECKER, Greifswald (DE); Birgit VOIGT, Greifswald (DE); Jorg FEESCHE, Erkrath (DE)

Correspondence Address: PAUL & PAUL, 2000 MARKET STREET, PHILADELPHIA, PA, 19103-3229, US

(21) Appl. No.: 11/764,926

(22) Filed: Jun. 19, 2007

Related U.S. Application Data
(63) Continuation of application No. PCT/EP05/13499, filed on Dec. 15, 2005.

(30) Foreign Application Priority Data
Dec. 22, 2004 (DE) 10 2004 061 664.7

(51) Int. Cl. C12Q 1/68; C12M 3/00

(52) U.S. Cl. 435/6; 435/287.2; 977/924

(57) ABSTRACT

The present application relates to nucleic acid-binding chips for monitoring bioprocesses, especially for detecting phosphate deficiency conditions. Said chips support probes for at least three of the following 47 genes: yhcR, tatCD, ctaC, gene for a putative acetoin reductase, spoIIGa, nasE, pstA, spoIIAA, gene for a hypothetical protein, yhbD, cotE, gene for a conserved hypothetical protein, yurl, spoVID, gene for a putative aromatic-specific dioxygenase, yhbE, gene for a putative benzoate transport protein, pstBB, spoIIIAH, gene for a hypothetical protein, spoIIQ, spoIIIAG, yvmA, gene for a putative ribonuclease, dhaS, yrbE, gene for a putative decarboxylase/dehydratase, htpG, yfkH, spoIIAB, spoIIIAF, alsD, gdh, yfkN, pstC, yfmQ, pstBA, dhaS homolog, gene for a putative phosphatase, phy, cypX, alsS, phoD, pstS, phoB, yvnA, yvmC, the total number of phosphate metabolism-specific different probes on the nucleic acid-binding chips not exceeding 100. The present application further relates to the use of corresponding gene probes, in particular on such chips, and to corresponding methods and possible uses.

### CROSS-REFERENCE TO RELATED APPLICATIONS

This is a § 365 continuation application of PCT/EP2005/013499 filed Dec. 15, 2005, which claims the priority of German patent application DE 10 2004 061 644.7, filed Dec. 22, 2004. Each of the foregoing applications is incorporated herein by reference.

### FIELD OF THE INVENTION

The present invention relates to nucleic acid-binding chips for monitoring bioprocesses, especially for detecting phosphate deficiency conditions and to the use of corresponding gene probes, in particular on said chips, and to methods and possible uses based on such probes.

### BACKGROUND OF THE INVENTION

The technical utilization of biological processes faces the very fundamental problem of monitoring the course thereof, in order to obtain the desired result, to save resources and/or to achieve an optimal result within a given period of time. Biological processes means, for example, culturing microorganisms on an agar plate or in a shaker culture, but in particular fermentation thereof, and obtaining raw materials by fermentation of microorganisms. There is an extensive prior art relating to this, with respect to both unicellular eukaryotes such as yeasts or streptomycetes and Gram-negative or Gram-positive bacteria.

The monitoring of such processes is carried out firstly by observing the properties and requirements of the observed organisms, which change during the course of said process, and this change is reflected, for example, in the optical density and viscosity of the medium, in gases taken up or released, in pH changes or in changing nutrient requirements. Measuring enzymatic activities via suitable assays, for example detecting activities of interest in the culture supernatant, may also be included here.

Secondly, various techniques have been developed over recent years in order to monitor the metabolic processes of the organisms in question at the level of gene expression. A common method for this is the use of genes for readily detectable proteins as indicators for the activity of the promoters of the actual genes of interest (promoter analysis, gene expression analysis). To this end, corresponding apparatuses (“(bio)sensors”) have also been developed.

Other techniques are concerned with the detection of the proteins of interest or of the mRNA coding for said proteins. These include (1.) proteom analysis, i.e. observing the change in provision of the cells in question with proteins, which is usually carried out by way of 2-dimensional gel electrophoresis of cell lysates, (2.) analysis of the mRNA formed (transcriptome) by way of an “genomic DNA array” generated in a similar way, and (3.) chip technology.

The latter is in a comparatively early stage of development. While the first two methods are based ultimately on quantitative isolations and time-consuming analyses of the macromolecules in question, chip technology is based on the principle of attaching probes for proteins or for nucleic acids on physically readable supports (chips), which probes react immediately to the presence of the proteins or nucleic acids in question. Compared to the former two technologies, chips of this kind promise to provide on-line analysis of the observed process. Another advantage is the requirement of comparatively small quantities of samples.

The principle of chip-based measurements is introduced, for example, diagrammatically in FIG. 2 of the article “Real-time electrochemical monitoring: toward green analytical chemistry” by J. Wang (Acc. Chem. Res., ISSN 0001-4842; Rec. Sept. 12, 2001, pages A-F). According to this, the sample to be analyzed is contacted with a biorecognition layer which may be, for example, enzyme, antibody, receptor of DNA; the signal received thereby is put out via a transducer, for example an amperometric or potentiometric electrode, by an amplifier (amplification/processing) as voltage or electric potential. The study in question also makes mention of optical systems which, with regard to miniaturizability and other advantages, appeared to be more favorable to the author than the electronically analyzable systems.

Owing to the present invention, the protein-specific chips may be put to one side. mRNA-recognizing chips are usually doped with complementary DNA molecules or DNA analogs. WO 95/11995 A1, for example, describes the preparation and utilization thereof for very detailed questions, such as, for example, the differentiation of point mutations. DNA-chip analyses include those with a PCR amplification of the target sequence and those without amplification. There are also those with optical evaluation of the signals caused by the recognition and those with electrical evaluation.

The optical detection methods partly require a mechanism of amplifying the signals. To this end, for example, fluorophores, acridinium esters or indirect detection via secondary binding processes, for example via biotin, avidin/streptavidin or digoxigenin, have been described. In the latter case, optical detection is carried out by using digoxigenin-specific antibodies which are labeled with an enzyme. Enzyme activity is then detected either colorimetrically or via luminescence. According to Westin et al. (2000), Nature Biotechnol., 18, pp. 199-204, hybridization can be coupled with a PCR on the DNA chip in order to be able to carry out the entire detection reaction on a chip (“lab on a chip concept”).

Other studies describe the development of DNA chips miniaturizing the principle of capillary electrophoresis for DNA sequencing or separation (Woolley and Mathies (1994), Proc. Natl. Acad. Sci., 91, pp. 11348-11352; Liu et al. (2000), Proc. Natl. Acad. Sci., 97, pp. 5369-5374).

Some publications have in principle already introduced electrically readable DNA chips (Hoheisel (1999), DECHEMA Jahresbericht [Annual Report] 1999, pp. 8-11; Hintsche et al. (1997), EXS, 80, pp. 267-283). Wright et al. (2000; Anal. Biochem., 282, pp. 70-79) utilize an ion channel sensor (ICS) for DNA detection, as has been described for the first time by Cornell et al. (1997; Nature, 387, pp. 580-583). This is a method in which the conductivity of molecular ion channels is detected by a binding reaction. The sensor is essentially an impedance element. According to Cheng et al. (1998; Nat. Biotechnol., 16, pp. 541-546), electric pulses may be utilized for amplifying the hybridization reaction on optical DNA chips. Fritsche et al. (2002; Laborwelt II) proposed an electrical chip system operating with metallic nanoparticles which are bound, for example, to oligonucleotides. In this system, a “metallic amplification” during the hybridization reaction causes a decrease in electric resistance on the electrode, which can then be measured as a signal.

Another approach is based on an electrical detection principle using DNA probes which, due to labeling by a suitable enzyme (for example alkaline phosphatase) result in an electrically active substrate after hybridization, which is then detectable by way of a redox reaction at the electrode (Hintsche et al. (1997), EXS, 80, pp. 267-283).

When the decision for a particular nucleic acid-recognizing chip type has been made with regard to the principle construction and the evaluation system, the more specific problem arises, as to which gene activities are to be observed. The fact that, for technical reasons, there are limits to the number of genes which can be analyzed simultaneously using one type of nucleic acid chip must be considered here. Thus, optically readable chips are currently superior to the electrically analyzable ones with regard to the number of probes which can be applied on the chip. The limits of the latter chips are set by the miniaturizability of the electronic measurement units.

Therefore the biological problem arises as to which selection of gene activities depicts the observed process in a suitable manner. This also includes monitoring product formation, if a product is produced, for example, fermentatively. It should at the same time also include control genes which indicate if the process develops in a direction which is not intended. For reasons of practicability, the number of different genes observed in the course of said monitoring should not be too high.

Biotechnological processes with Gram-positive bacteria are of particular industrial interest, since said bacteria are employed for industrial production of valuable substances particularly due to their secretion ability. Among these, those of the genus Bacillus, and among those in turn the species B. subtilis, B. amyloliquefaciens, B. agaradherens, B. licheniformis, B. lentus and B. globigil, are currently most important economically.

The studies introduced hereinbelow, for example, are concerned with the simultaneous observation of the activity of multiple genes in bacteria (multiparameter recording). The article “Monitoring of genes that respond to process-related stress in large-scale bioprocesses” by Schweder et al. (1999), Biotech. Bioeng., 65, pp. 151-159 describes the change in mRNA levels of various stress factor-inducible genes, namely clpB, dnaK (induced by heat shock), uspA (glucose deficiency), proU (osmotic stress), pfl and frd (O₂ deficiency) and ackA (excess glucose) in the course of an E. coli fermentation and in the subsequent concentration phase. They were recorded by a PCR-based method carried out in the usual manner. Here, different rates of expression were found already at various sites of the reactor and reactions within seconds to altered conditions were found.

Another fermentation of E. coli is described in the study “Monitoring of genes that respond to overproduction of an insoluble recombinant protein in Escherichia coli glucose-limited fed-batch fermentations” by Jürgen et al. (2000), Biotech. Bioeng., 70, pp. 217-224. This study observes expression of the genes Ion, dnaK, ibpB, htrA, ppiB, groEL, tig, s6, I9 and dps, partly at the mRNA level, partly at the protein level and partly at both levels. The study was carried out by way of 2D PAGE and DNA array technique. In view of the results it is suggested to monitor recombinant bioprocesses such as heterologous protein production via (directly) process-relevant proteins and reporter genes such as ibpB.

The study “Genomic analysis of high-cell-density recombinant Escherichia coli fermentation and “cell conditioning” for improved recombinant protein yield” by R. T. Gill et al. (2001; Biotech. Bioeng., 72, pp. 85-95) is concerned with another observation of the fermentation process during expression of a recombinant protein by E. coli. It describes the fact that the stress genes degP, uvrB, alpA, mltB, recA, ftsH, ibpA, aceA and groEL are expressed more strongly at high cell density compared to low cell density under said conditions. Said genes formed groups of certain clusters according to the strength of the reaction. This was determined by an approach based on RT-PCR and DNA microarray, which was supplemented by dot-blot analysis and which was applied to samples from two points in time of the fermentation, namely at the start, at low cell density, and toward the end, at high cell density. From this, cell conditioning approaches were developed in order to reduce the stress response of the cells.

Fundamental differences in the expression patterns of Gram-positive organisms to those of Gram-negative bacteria are uncovered in the study “Proteome and transcriptome based analysis of Bacillus subtilis cells overproducing an insoluble heterologous protein” by Jürgen et al. (2001), Appl. Microbiol. Biotechnol., 55, pp. 326-332. Here, expression of, inter alia, the genes dnak, groEL, grpE, clpP, clpC, clpX, rpsB and rplJ in B. subtilis are described, as can be determined by the DNA-macroarray technique, or by two-dimensional polyacrylamide gel electrophoresis. According to this, the genes for purine and pyrimidine syntheses and those of particular ribosomal proteins are expressed more strongly in Gram-positive bacteria employed for overexpression than was expected on the basis of the findings for Gram-negative bacteria. Another difference relates to the proteases Lon and Clp.

Several publications have meanwhile disclosed or at least indicated the possible preparation of some of these genes or even of nucleic acid-binding chips containing some of these genes. Thus, for example, the two patent applications DE 10136987 A1 and DE 10108841 A1 disclose in each case a Corynebacterium glutamicum gene, namely clpC and citB, respectively. Both genes are described as being relevant to the amino acid metabolism, and this is the reason for a commercially interesting utilization of said genes which is said to comprise inactivating or at least attenuating them in order to optimize fermentative production of amino acids by said microorganism. According to said applications, further possible applications may comprise providing probes for the gene products in question on nucleic acid-binding chips.

On the other hand, more and more genomic data of various organisms are published, which contain such an abundance of sequence data that a representative selection therefrom seems desirable. Thus, the patent application WO 02/055655 A2 discloses more than 1800 DNA sequences which have been determined by completely sequencing the genome of the microorganism Methylococcus capsulatus.

Meanwhile, for example, the complete genome of the Gram-positive Bacillus licheniformis has also been sequenced. It is described in the publication “The Complete Genome Sequence of Bacillus licheniformis DSM13, an Organism with Great Industrial Potential” (2004) by B. Veith et al. in J. Mol. Microbiol. Biotechnol., Volume 7(4), pages 204 to 211 and, in addition, is accessible under the entry AE017333 (bases 1 to 4 222 645) in the GenBank database (National Center for Biotechnology Information NCBI, National Institute of Health, Bethesda, Md., USA; http://www.ncbi.nlm.nih.gov; as of 12.2.2004).

Using the technique of optically analyzable chips, it is meanwhile even possible to prepare nucleic acid-binding chips which cover virtually a complete genome or the corresponding transcriptor (genomic DNA chips).

The application WO 2004/027092 A2 provides a representative cross section with a manageable number of genes, in order to identify various physiological states which an observed microorganism can go through during culturing. These include, for example, starvation conditions with regard to various nutrients or stress situations such as, for example, heat or cold shock, shearing stress, oxidative stress or oxygen limitation. Said genes are as follows: acoA, ahpC, ahpF, citB, clpC, clpP, codY, cspA, cspB, des, dnak, eno, glnR, groEL, groL, gsiB, ibpA, ibpB, katA, katE, lctP, ldh, opuAB, phoA, phoD, pstS, purC, purN, pyrB, pyrP, sigB, tnrA, trxA and ydjF. This application also reveals the corresponding DNA sequences from B. subtilis, E. coli and/or B. licheniformis. As a result, it has become possible to prepare also corresponding nucleic acid-binding chips which, when monitoring a bioprocess based on microorganisms, in particular Gram-positive or Gram-negative bacteria, indicate changes in the metabolic activities which characterize said process.

Nucleic acid-binding chips based on this selection of genes provide a certain, but overall rather only coarse, overview of the particular readable situation. They are usually not able to specifically illuminate an individual partial problem; however, an individual positive signal can result from various situations or else be only false-positive, and it is therefore often—and in particular in such an unclear situation—sensible to analyze a selected metabolic aspect separately. On the other hand, especially with electrically readable nucleic acid-binding chips which have the advantage of on-line analysis, the number of simultaneously occupiable places is limited so that it is not possible to simply apply additional gene probes for recording additional, special metabolic situations.

Particular metabolic situations, and among them even deficiency conditions, are utilized in biotechnology. Thus, the application DE 10012283 A1 discloses an application utilizing the inducibility of genes by phosphate defiency. The study described therein uses the promoters of the B. subtilis genes pstS, phoD, phoB or glpQ for the regulation of transgenes which are to be activated by Gram-positive host bacteria for heterologous gene expression. For this, the B. subtilis PhoP-PhoR regulatory system must be made available at the same time in order to activate the relevant promoters. According to this application, it is intended to artificially cause phosphate deficiency in order to obtain induction of the promoter chosen in each case via PhoP and PhoR. Thus, it is not assumed that intrinsic regulatory systems of the cell are capable of inducing the artificially introduced promoters of the B. subtilis genes pstS, phoD, phoB and glpQ. Thus it is known, for example, from other studies (not shown here) that the pho genes in B. licheniformis are organized differently, i.e. also regulated differently.

However, as explained above, it is usually not desired to expose the cells to a stress situation in the course of a bioprocess, since phosphate deficiency in particular is a metabolic situation which may be critical for microorganisms and therefore limiting to a corresponding bioprocess.

There is therefore a particular need for carrying out a chip-based on-line analysis on this matter and for being able to intervene in the running bioprocess on time and therefore even more specifically, owing to the result which can be quickly obtained by said analysis. This prevents a loss of yield which would result from a phosphate bottleneck which is recognized too late or not at all.

The prior art described further/other genes whose expression is increased during phosphate deficiency, albeit not at the same level in all of the microorganisms relevant to biotechnology. Thus, the publication by Ishige et al. in J. Bacteriol., Volume 185 (No. 15), pages 4519 to 4529, deals with a DNA microarray analysis of the genes activated by phosphate deficiency (the “phosphate stimulon”) in Corynebacterium glutamicum. In this study, the stimulus is caused by going from orthophosphate as the sole phosphate source to a state of phosphate deficiency. This clearly induces some genes associated with phosphate metabolism, in agreement with the data for other microorganisms. In contrast, the induction of further genes is attributed to mere growth effects. It was also observed that only a few rather than all of the known phosphate metabolism genes are induced. Conversely, some proteins are increasingly produced whose homologs in other microorganisms are not increasingly expressed following said stimulus, for example a nucleotidase whose homolog in E. coli has an unaltered level of expression, and a ferritin-like protein and also a probable extracellular nuclease, NucH, whose levels of expression are not significantly elevated in B. licheniformis, for example (data not shown).

The publication by Antelmann et al. in J. Bacteriol., Volume 182 (No. 16), pp. 4478 to 4490 investigates the proteins inducible by phosphate deficiency in B. subtilis at the proteome and transcriptome levels with the aid of two-dimensional gel electrophoresis; the microarray technology is here discussed merely as a to some extent possibly supplementing technology. FIG. 4 discloses ten proteins in total which are increasingly produced in this organism upon a phosphate deficiency stimulus. The strongest signals are those of GlpQ, PhoD and PstS, followed by PhoB and Pel. In contrast to this, some studies in connection with the present application found that glpQ and pel in B. licheniformis are only negligibly overexpressed under phosphate deficiency. Instead, for example, the phytase gene in B. licheniformis (see examples) produced a surprisingly strong signal, which is not the case in B. subtilis.

Thus there are several fundamental difficulties with the idea of designing RNA-recognizing chips suitable for monitoring bioprocesses. Firstly, with each gene, there is the question of transferability to other organisms: genes must be selected which produce distinct signals in as many microorganisms as possible that are relevant to such bioprocesses. Secondly, there is the question of specificity: the strong signals must also be assignable very clearly to the metabolic situation in question. Those signals which respond to a plurality of different metabolic situations and/or are general stress signals should be excluded as much as possible.

### SUMMARY OF THE INVENTION

The approach to a solution chosen for the present invention consists of making a selection of a whole number of genes which is as representative as possible, with usually not all, but according to the invention a plurality, of the probes giving signals in the organism observed in each case. On the other hand, those genes which give likewise strong or even stronger signals in metabolic situations other than that of phosphate deficiency should be excluded.

The object of the present invention is therefore that of identifying genes which can be linked as clearly as possible to the stress signal of phosphate deficiency in organisms, in particular microorganisms. It was the aim to develop probes for these genes in order to be able to employ them for monitoring corresponding bioprocesses.

This should enable nucleic acid-binding chips to be occupied with gene probes for some or a plurality of these genes, whereby nucleic acid-binding chips can be obtained which indicate reliably the signal “phosphate deficiency” in the course of a monitored bioprocess (phosphate deficiency sensors). This was the object in particular for those nucleic acid-binding chips whose number of occupiable places is comparatively low due to their design, in particular the electrically analyzable chips, since these, on the other hand, have the advantages of rapid readability and therefore make an on-line analysis possible. This ensures early intervention, where appropriate, in order to optimize the bioprocess in question with respect to phosphate supply.

A DNA-binding chip of this kind should be usable for a plurality of comparable processes and be able to be adapted to specific possible uses with comparatively small variations. It should preferably be directed to bioprocesses on the basis of Bacillus species, in particular B. subtilis, B. amyloliquefaciens, B. lentus, B. globigii, and very particularly to B. licheniformis. Among bioprocesses, fermentations were to the fore, in particular industrial production of products, very particularly of overexpressed proteins.

Such a phosphate deficiency sensor should also make possible corresponding methods of measuring the physiological state of the observed cells and also corresponding possible uses for monitoring the observed biological processes.

Said object was solved by studying a multiplicity of genes from the biotechnologically important bacterium B. licheniformis with respect to their activatability by transfer of the culture in question to a state of phosphate deficiency (Example 1). In this connection, it was surprisingly found that by no means all genes involved in phosphate metabolism produce a clear signal in this respect. In addition—and also surprisingly—an activation of those genes was observed which have previously not necessarily been readily linked to phosphate metabolism, for example sporulation genes; according to the invention, these should now, independently of their previously known function, likewise be regarded as phosphate metabolism genes. Both points are covered in Example 2 of the present application. In this connection, inductions of very different strength were observed. According to the teaching of the present invention, the suitability of genes as indicators should increase as a function of the strength of this response. According to the invention, those genes which give a distinct signal which is significantly above a particular threshold level are therefore selected as phosphate deficiency indicators. The further the result exceeds this level, the more they are preferred according to the invention, and this explains a corresponding grading with respect to preferred aspects of the invention. At the same time, those genes which have likewise produced strong or even stronger signals in situations other than that of phosphate deficiency were removed again (data not shown).

Table 1 depicts all 235 genes of Bacillus licheniformis DSM13, which were determined in Example 1 and whose induction under phosphate deficiency was observed, with a factor of at least three being considered significant. Of these, Table 2 lists all 47 genes whose induction by phosphate deficiency has been at least a factor of 10 at any measured point in time and for which it was possible, from parallel studies not shown herein, to assume that they were comparatively specific for said signal. These genes are listed again in Table 3 with respect to the strength of their observed maximum induction. Their DNA and amino acid sequences are listed in the sequence listing of the present application, with the odd numbers representing DNA sequences and the subsequent even numbers representing the amino acid sequences derived in each case. The respective SEQ ID numbers in Tables 2 and 3 also refer to these sequences. The genes are as follows, in the order of decreasing strength of the induction caused by phosphate deficiency (compare Tables 2 and 3):
yvmC (similar to proteins of unknown function; SEQ ID No. 75, 76);
yvnA (similar to proteins from B. subtilis; SEQ ID No. 77, 78);
phoB (alkaline phosphatase III; SEQ ID No. 21, 22);
pstS (phosphate ABC transporter/binding protein; SEQ ID No. 47, 48);
phoD (phosphodiesterase/alkaline phosphatase; SEQ ID No. 23, 24);
alsS (alpha-acetolactate synthase; SEQ ID No. 29, 30);
cypx(cytochrome P450-like enzyme; SEQ ID No. 3, 4);
phy (phytase; SEQ ID No. 33, 34);
gene for a putative phosphatase (SEQ ID No. 61, 62);
dhaS homolog (DhaS aldehyde dehydrogenase homolog; SEQ ID No. 17,18);
pstBA (phosphate ABS transporter; SEQ ID No. 87, 88);
yfmQ (unknown function; SEQ ID No. 69, 70);
pstC (phosphate ABC transporter/permease; SEQ ID No. 91, 92);
yfkN (similar to 2′,3′-cyclo-nucleotide 2′-phosphodiesterase; SEQ ID No. 11,12);
gdh (glucose 1-dehydrogenase; SEQ ID No. 31, 32);
alsD (alpha-acetolactate decarboxylase; SEQ ID No. 27, 28);
spoIIIAF (sporulation factor III AF; SEQ ID No. 79, 80);
spoIIAB (anti-sigma F factor/stage II sporulation protein AB; SEQ ID No. 37, 38);
yfkH (similar to proteins; SEQ ID No. 67, 68);
htpG (class III heat-shock protein; SEQ ID No. 1, 2);
gene for a putative decarboxylase/dehydratase (SEQ ID No. 57, 58);
yrbE (similar to dehydrogenase; SEQ ID No. 9,10);
dhaS (aldehyde dehydrogenase; SEQ ID No. 19, 20);
gene for a putative ribonuclease (SEQ ID No. 93, 94);
yvmA (similar to multidrug transporter SEQ ID No. 51, 52);
spoIIIAG (sporulation factor III AG; SEQ ID No. 81, 82);
spoIIQ (sporulation factor II Q; SEQ ID No. 43, 44);
gene for a hypothetical protein (SEQ ID No. 63, 64);
spoIIIAH (sporulation factor III AH; SEQ ID No. 83, 84);
pstBB (phosphate ABC transporter/ATP-binding protein; SEQ ID No. 89, 90);
gene for a putative benzoate transport protein (SEQ ID No. 49, 50);
yhbE (similar to proteins from B. subtilis; SEQ ID No. 73, 74);
gene for a putative aromatics-specific dioxygenase (SEQ ID No. 55, 56);
spoVID (sporulation factor VI D; SEQ ID No. 45, 46);
yurl (similar to ribonuclease; SEQ ID No. 15,16);
gene for a conserved hypothetical protein (SEQ ID No. 59, 60);
cotE (outer spore coat protein; SEQ ID No. 39, 40);
yhbD (similar to proteins from B. subtilis; SEQ ID No. 71, 72);
gene for a hypothetical protein (SEQ ID No. 65, 66);
spoIIAA (anti-sigma F factor antagonist/stage 11 sporulation protein AA; SEQ ID No. 35, 36);
pstA (phosphate ABC transporter/permease; SEQ ID No. 85, 86);
nasE (subunit of assimilatory nitrite reductase; SEQ ID No. 7, 8);
spoIIGA (sporulation factor 11 GA; SEQ ID No. 41, 42);
gene for a putative acetoin reductase (SEQ ID No. 53, 54);
ctaC (cytochrome CAA3 oxidase/subunit 11; SEQ ID No. 5, 6);
tatCD (component of the twin-arginine translocation pathway; SEQ ID No. 25, 26);
yhcR (similar to 5′-nucleotidase; SEQ ID No. 13,14).

One solution to the stated object is a nucleic acid-binding chip doped with probes for at least three of the following 47 genes: yhcR, tatCD, ctaC, gene for a putative acetoin reductase (SEQ ID No. 53 homolog), spoIIGA, nasE, pstA, spoIIAA, gene for a hypothetical protein (SEQ ID No. 65 homolog), yhbD, cotE, gene for a conserved hypothetical protein (SEQ ID No. 59 homolog), yurl, spoVID, gene for a putative aromatic-specific dioxygenase (SEQ ID No. 55 homolog), yhbE, gene for a putative benzoate transport protein (SEQ ID No. 49 homolog), pstBB, spoIIIAH, gene for a hypothetical protein (SEQ ID No. 63 homolog), spoIIQ, spoIIIAG, yvmA, gene for a putative ribonuclease (SEQ ID No. 93 homolog), dhaS, yrbE, gene for a putative decarboxylase/dehydratase (SEQ ID No. 57 homolog), htpG, yfkH, spoIIAB, spoIIIAF, alsD, gdh, yfkN, pstC, yfmQ, pstBA, dhaS homolog (DhaS aldehyde dehydrogenase homolog; SEQ ID No. 17 homolog), gene for a putative phosphatase (SEQ ID No. 61 homolog), phy, cypx, alsS, phoD, pstS, phoB, yvnA, yvmC, with the total number of all phosphate metabolism-specific different probes not exceeding 100.

Detailed information about these genes can be found in Examples 1 to 3 and in Tables 1 to 3 of the present application. The sequence listing discloses said genes as obtainable from B. licheniformis DSM 13. Most of these have also been described from other species (see below). Some, however, are putative (suspected) genes or genes coding for putative (suspected) enzymes. These are defined according to the invention as far as possible, owing to database comparisons, as those having a putative function and in addition as homologs of the B. licheniformis genes found. Two points must be explained in this context:
firstly, a biochemical analysis of some or other genes could find that said putative function does not correspond to the actual function. In this case the putative function is not held onto. Rather, these findings do not question the invention insofar as, according to the invention, only the observation of increased transcription in connection with the phosphate deficiency matters, so that the gene activity in question may well serve as an indicator for phosphate deficiency independently of the ultimately exerted enzyme activity.
secondly, in the absence of a gene name, it was not possible to find a more suitable definition for said genes than that of the gene itself. As a result, said genes are referred to as homologs. Thus the homologous genes in other species than B. licheniformis can be assumed to be activated under phosphate deficiency. Should it turn out that in an observed species a plurality of homologs of any of these genes exist, which are capable of transcript formation in vivo, the information “SEQ ID No. . . . homolog” then refers to in each case the most similar of these different possible genes.

According to the invention, at least three of these genes are selected in order to obtain as reliable a message as possible, i.e. in order to rule out an individual false-positive signal caused by only one type of probe.

According to the invention, a nucleic acid-binding chip means any subject matters which are provided with nucleic acid-specific probes and which produce in each case an analyzable signal upon binding of one or more specifically recognized nucleic acids.

### BRIEF DESCRIPTION OF THE FIGURE

FIG. 1: is a diagrammatic representation of on-line monitoring of a bioprocess with electrical DNA chips of the invention

### DETAILED DESCRIPTION

On-line monitoring of the bioprocess is advantageously carried out by way of the following steps:
1. sampling, for example from fermentation of a microorganism;
2. cell disruption by routine methods;
3. RNA isolation by routine methods;
4. hybridization on a chip charged according to the invention with nucleic acids (for example DNA) or nucleic acid analogs (for example structurally similar compounds which are difficult to hydrolyze);
5. recording the electric signals of an appropriately constructed electro-chip; alternatively, the recording of optical signals from an optical DNA chip would also be possible;
6. preferably computer-assisted data evaluation.

With the current state of development, the use of electrical chips results in an approximate total analysis time of less than 2 h, with the use of conventional optical DNA chips resulting in a time of approx. 12 h.

The designing of chips doped with nucleic acids as probes is known from the prior art illustrated at the outset. In principle, all of them may be utilized for embodiments of the present invention. They are based on the principle of nucleic acid hybridization of the mRNA to be detected (or of a molecule derived therefrom) with the probe presented on the chip. Depending on the system for evaluating the signal caused by said hybridization, a distinction is made between chips with an optical and with an electrical analytical system. According to the invention, both systems are applicable in principle.

Such chips are used as follows for controlling (monitoring) the bioprocess observed in each case: a sample containing the biological material to be analyzed is removed from the process at a particular time. RNA, in particular mRNA, is isolated from said material by methods known per se, for example with cell disruption and the use of a denaturing buffer. Said RNA is labeled itself or used as a starting molecule for a molecule introduced to the measurement (for example cDNA obtained by reverse transcription), and the molecules obtained are advantageously passed in a buffer across/through the chip. Hybridization (sandwich labeling) of a prepared RNA or the derivative thereof with the homologous (i.e. congruent with respect to its sequence) probe provided on the chip (target nucleic acid, for example target DNA or target nucleic acid analog) results in a corresponding optically or electronically analyzable signal. The latter is based, for example, on the labeling of the binding mRNA or a transcript thereof with a chromogenic or fluorescent marker, a hybridization with a second probe or on a secondary detection reaction, for example via RT-PCR.

Since usually in each case multiple molecules of the same probe are bound to the chip, the strength of the hybridization signal is, over a certain range—which can be optimized in the individual case, where appropriate—, proportional to the number of specific mRNA present in the sample at the time of sampling. In this way, the strength of the signal is a direct measure of the activity of the gene in question at the time of sampling.

The time interval between sampling and measurement should be kept here as short as possible, for example by a substantially automated sampling, processing thereof and passing thereof across/through the sensor.

Suitable organisms observed (monitored) with the aid of a chip of the invention are in principle any plants, animals and microorganisms, in particular those which are utilized commercially. Thus, for example, the application DE 19860313 A1 entitled “Verfahren zur Erkennung und Charakterisierung von Wirkstoffen gegen Pflanzen-Pathogene” [Method of recognizing and characterizing active compounds for plant pathogens] reveals metabolic situations in plants, in particular crops, which must be observed. It is also possible to observe, for example, useful animals or laboratory animals. Eukaryotic cell cultures are quite interesting commercially, for example for producing monoclonal antibodies, and in particular for fermentative production of food, for example by alcoholic fermentation carried out by yeasts. Bacteria are utilized in particular for industrial production of proteins or low molecular-weight variable substances (biotransformation), for example vitamins or antibiotics.

According to the invention, probes mean any molecules capable of interacting in each case substantially specifically with nucleic acids (binding them). This interaction is utilized according to the invention in order to obtain a substantially unambiguously assignable, analyzable signal within the framework of a corresponding arrangement (chip).

From a chemical point of view, a probe of the invention is usually a compound capable of binding mRNA molecules or nucleic acids derived therefrom via hydrogen bonds, as is the case, for example, also for the interaction of the two strands of a DNA or for DNA-RNA interaction. Said compound may be, for example, a DNA which is more stable to hydrolysis than RNA.

In addition, the prior art has disclosed further molecules, in particular chemically synthesized ones, which biomimetically make possible the same interaction but are more stable than DNA, for example due to the fact that the phosphate ester bonds of the backbone have been replaced with less hydrolytically sensitive bonds. Such nucleic acid analog probes characterize preferred embodiments of the present application (see below). The respective specific probes would have to be synthesized correspondingly, for example according to the model of the sequence listing related to this application. This fits in with the aspect that chips of the invention should advantageously be usable several times, in particular during a single observed process in the course of which constant monitoring is desirable.

Limiting to the usability of a probe is in each case the extent of homology between the provided probe and the mRNA or the nucleic acid derived therefrom, which is to be recognized via hybridization. Ultimately, the extent of hybridization of the probe with the mRNA to be detected (see above) decides its usability as a probe and must, in the individual case, be optimized experimentally and/or taken into account by adapting the evaluation of the signal. Under the conditions determined by the construction of the measuring apparatus and other influences, a hybridization must take place which can be specifically attributed only to the gene of interest, is sufficiently strong in order to give a positive signal, and, on the other hand, is not too strong for the recognized molecule to diffuse off again after generation of the signal, in order to empty the binding site for the next molecule or to enable the signal to decay; the latter, where appropriate, via a corresponding washing step.

However, it is necessary, prior to using chips of the invention for an organism of interest, to estimate the extent of homology between the genes in question, then, if the affinity of the mRNAs to be detected for the presented probes is insufficient, to anchor such probes of the same genes of the invention from more closely related species on the chip and to carry out calibration measurements in order to obtain reliable information as to which signal strength corresponds to which concentration of special mRNA.

The identification of the 47 genes essential to the present invention is described in the examples of the present application. The sequences thereof obtainable from Bacillus licheniformis are indicated in the sequence listing of the present application (SEQ ID No. 1 to 94), wherein the odd-numbered sequences are DNA sequences and the sequences one number up are in each case the amino acid sequences derived therefrom. While the DNA sequences can be utilized immediately for preparing probes (see above), the amino acid sequences are used, for example, for checking gene function via sequence database comparisons and may further be used for generating similar nucleic acid-recognizing probes, for example by back translating the genetic code.

As illustrated in Example 1, numerous different gene transcripts, i.e. mRNA molecules, were studied, in particular those which were generally known to be involved in phosphate metabolism. The mRNA molecules were isolated at various points in time during the transition of B. licheniformis DSM 13 to a phosphate deficiency condition. Example 1 likewise describes the way in which the increase in concentration of said mRNA inside B. licheniformis cells was determined experimentally. Possible alternative determinations of this might be established in the prior art; the compilation in Table 1 (Example 2) is critical to the understanding of the present invention. It depicts the changes in the concentrations of 235 mRNAs in total linked to the transition. In this connection, the following thresholds of the ratio of the amount of RNA of the particular gene to the control value were considered significant: according to the invention, the genes whose RNA has a ratio of >3 (i.e. at least a three-fold increase) are considered induced; a distinct induction is present at a ratio of >10; genes having an RNA ratio of <0.3 (i.e. a decrease to less than 30%) are distinctly repressed. For the 235 genes listed in Table 1, at least a three-fold increase was observed at any of the observed points in time.

As Table 2 proves, there are surprisingly among these 235 genes only 47 genes having an at least 10 fold induction at any of the observed points in time under the conditions of phosphate deficiency described in Example 1. According to the invention, these 47 genes are considered representative indicators of a phosphate deficiency condition. Further information about these genes, for example about their function or deviating start codons, can be found in Tables 2 and 3 and the sequence listing; the particular English names for the corresponding proteins have already been listed above, in the order of the information in Table 3.

All of these genes have been described in each case individually in the prior art. They can be found in generally accessible databases for the various organisms. As mentioned above, the sequences indicated in the sequence listing for B. licheniformis DSM 13 have been determined from this microorganism and are virtually identical to the information described in the publication “The Complete Genome Sequence of Bacillus licheniformis DSM13, an Organism with Great Industrial Potential” (2004) by B. Veith et al. in J. Mol. Microbiol. Biotechnol., Volume 7(4), pages 204 to 211 and additionally accessible under the entry AE017333 (bases 1 to 4 222 645) in the GenBank database (see above). The B. licheniformis DSM 13 strain is generally obtainable via Deutsche Sammlung von Mikroorganismen und Zellkulturen GmbH, Mascheroder Weg 1b, 38124 Braunschweig, Germany (htt://www.dsmz.de). It has the deposition number ATCC 14580 at the American Type Culture Collection, 10801 University Boulevard, Manassas, Va. 20110-2209, USA (http;//www.atcc.org).

A large proportion of the genes from other organisms, which correspond to the 47 genes mentioned, have likewise been deposited in generally accessible databases, for example for the well-characterized species B. subtilis and E. coli which are generally regarded as model organisms of Gram-positive and Gram-negative bacteria, respectively. The corresponding sequences may be found, for example, in the databases of Institut Pasteur, 25, 28 rue du Docteur Roux, 75724 Paris CEDEX 15, France, which are accessible via the internet addresses http://genolist.pasteur.fr/Colibri/ (for E. coli) and http://genolist.pasteur.fr/Colibri/ (for B. subtilis), respectively (as of 12.2.2004). Other databases suitable for this are that of the EMBL-European Bioinformatics Institute (EBI) in Cambridge, United Kingdom (http://www.ebi.ac.uk), Swiss-Prot (Geneva Bioinformatics (GeneBio) S.A., Geneva, Switzerland (http://www.genebio.com/sprot.html) or GenBank (National Center for Biotechnology Information NCBI, National Institutes of Health, Bethesda, Md., USA).

Said “corresponding genes” mean those which in each case code for the proteins which catalyze the same chemical reaction in the observed organism or which are involved in the same physiological process as the mentioned 47 proteins in B. licheniformis DSM 13. Most of these have for other organisms similar names and abbreviations to those indicated for B. licheniformis in Tables 1 and 2 because said names represent the particular function. If in doubt, they can be recognized via their sequence which in each case is the next similar (most homologous) from the organism in question to the sequences mentioned herein. When assigning the function, the similarity of the amino acid sequences to one another is especially decisive because the amino acids are the carriers of the function of the protein and, owing to the degeneracy of the genetic code, various nucleotide sequences can code for the same amino acid sequence.

Particularly high degrees of relationship exist between closely related species. Thus it can be assumed in principle that for most of the 47 genes mentioned homologs can be found in all species, even in cyanobacteria, in eukaryotic cells such as, for example, fungi, or in Gram-negative species such as E. coli or Klebsiella. This probability is even higher for Gram-positive bacteria, in particular of the genus Bacillus, because B. licheniformis DSM 13 from which the sequences listed in the sequence listing are derived is such a Gram-positive bacterium. It can moreover be assumed that in increasingly related organisms the homologous genes are also increasingly subjected to the same regulatory mechanisms or regulatory mechanisms acting in the same way; therefore, these homologs should also indicate the same metabolic situation, in particular a phosphate deficiency. In this respect, B. licheniformis is a good choice of an exemplary organism because the commercially likewise particularly important species B. subtilis, B. amloliquefaciens, B. lentus, B. globigii are likewise Bacilli and therefore Gram-positive. This is in accordance with the aspect of the stated object in this regard.

It should be noted that, in order to practice the invention for a particular species, not all of the 47 genes mentioned must be known but that only a few of them (see below) are sufficient in order to reproduce phosphate metabolism and, in particular, to be able to detect the transition to a phosphate deficiency condition. Nevertheless, the reliability of the information about the phosphate supply state increases with an increasing number of probes. If a plurality of genes are known that appear to be suitable in principle on the basis of the present disclosure, it is recommended to carry out an expression study prior to preparing a corresponding chip, in order to check, whether the genes in question actually allow significant information, similarly to the representation in Example 1 or else via a Northern analysis. Shifts regarding the level of expression caused by phosphate deficiency may be more likely the less related the observed species is to B. licheniformis, so that subgroups (where appropriate other subgroups than the ones listed below) of these 47 genes prove to be particularly suitable and therefore preferred.

Thus the preparation of a nucleic acid-binding chip of the invention for an organism not specified herein must involve identifying the homologous genes corresponding to at least some of the genes specified for B. licheniformis, for example by comparing the known DNA sequences of the organism in question with the sequences indicated herein. These or parts thereof (see below) may then serve per se as probes or as a template for synthesizing corresponding probes which are applied to a nucleic acid-binding chip by methods known per se.

If it is the case that some homologous sequences have not been deposited in databases, it is possible for the skilled worker to synthesize particular probes on the basis of the sequences disclosed in the sequence listing of the present application, in order to screen with the aid thereof a gene library generated for the desired organism (genomic or preferably on the basis of the cDNA) for the homolog in question by common methods. As an alternative to this, it is also possible to synthesize on the basis of the DNA sequences indicated in the sequence listing oligonucleotides which serve as PCR primers in order to amplify the genes in question or parts thereof that can be used as probes from a DNA preparation of the complete genome or from a cDNA preparation of the organism of interest. These or parts thereof (see below) may be employed as probes on nucleic acid-specific chips of the invention.

An essential feature of the present invention is the fact that the total number of all the different phosphate metabolism-specific probes does not exceed 100. This feature correlates with the stated object, according to which it should mainly focus on those nucleic acid-binding chips whose number of occupiable places is comparatively low due to their design. These are in particular the electrically analyzable chips.

Increasing preference is therefore given to the total number of all the different phosphate metabolism-specific probes not exceeding 100, 95, 90, 85, 80, 75, 70, 65, 60, 55 or 50.

This may also include probes for other genes not discussed in the present application which are used for control purposes, for example those which are expressed only with adequate phosphate supply. The disappearance of a signal attributable thereto may likewise indicate the transition to the state of phosphate deficiency. If such a signal were to be retained, then this would serve as the control of the reliability of the phosphate deficiency signal to be determined according to the invention.

Further phosphate metabolism-specific probes may include, for example, those which are induced by an excess of phosphate, and may include also others which appear not to be directly associated with phosphate metabolism, but which may be defined as such due to said inducibility. As a result, such a chip also produces analyzable information useful in the observed process, after phosphate deficiency has been overcome, for example by carrying out appropriate countermeasures.

Nucleic acid-specific probes are furthermore usually in each case only fragments of the complete genes (see below). In individual cases, for example with regulation via splicing or with large, multifunctional polypeptides, it may therefore be sensible to detect the same gene with two or more different probes. Corresponding embodiments are therefore, where appropriate, characterized by more than 47 probes which, however, do not respond to more than said 47 genes.

Depending on the process to be observed, probes for further genes or gene products may also be present on chips of the invention (see below).

On the other hand, the core of the invention consists specifically of the specificity of the chip in question, with which a special metabolic situation is to be recorded. In the case of such a specific question, the preparation of a chip with more than 100 probes responding to various genes, or even of a chip which reproduces a large part of the genome of an organism, is not part of the invention described herein, due to the complexity associated therewith. Rather, it is possible to use both kinds of chips in an observed bioprocess in a useful manner in parallel: thus it is possible for the chips containing numerous various gene probes or a representative cross section of various, possibly relevant situations, as they are provided by the application WO 2004/027092 A2, to provide a rough overview of the condition of the organism in question, while a chip of the invention is used as a control, if there is reason to be concerned about the possibility of the cells in question entering a phosphate deficiency state.

A preferred embodiment is a nucleic acid-binding chip of the invention, which is doped increasingly preferably with the probes indicated above in the order indicated there.

This means the genes listed in Table 3 in reverse order. Accordingly, chips with probes for the yhcR gene (similar to 5′-nucleotidase; SEQ ID No. 13, 14) still are least preferred among the chips of the invention with respect to gene selection for the formation of probes, since this gene has the weakest induction among the 47 genes mentioned, whose transcription is enhanced in a significant manner due to phosphate deficiency. In contrast, most preference with respect to gene selection is given to those chips with a probe for the yvmC gene (similar to proteins of unknown function; SEQ ID No. 75, 76). This is because this gene is induced by a factor of nearly 150 over the starting level and therefore has the highest induction of all the genes measured. The signal linked thereto should thus be the most suitable of all the genes examined for indicating the metabolic situation “phosphate deficiency”.

A preferred embodiment is a nucleic acid-binding chip of the invention, wherein at least three of the probes are selected from the following 39 genes: gene for a hypothetical protein (SEQ ID No. 65 homolog), yhbD, cotE, gene for a conserved hypothetical protein (SEQ ID No. 59 homolog), yurl, spoVID, gene for a putative aromatic-specific dioxygenase (SEQ ID No. 55 homolog), yhbE, gene for a putative benzoate transport protein (SEQ ID No. 49 homolog), pstBB, spoIIIAH, gene for a hypothetical protein (SEQ ID No. 63 homolog), spoIIQ, spoIIIAG, yvmA, gene for a putative ribonuclease (SEQ ID No. 93 homolog), dhaS, yrbE, gene for a putative decarboxylase/dehydratase (SEQ ID No. 57 homolog), htpG, yfkH, spoIIAB, spoIIIAF, alsD, gdh, yfkN, pstC, yfmQ, pstBA, dhaS homolog (DhaS aldehyde dehydrogenase homolog; SEQ ID No. 17 homolog), gene for a putative phosphatase (SEQ ID No. 61 homolog), phy, cypX, alsS, phoD, pstS, phoB, yvnA, yvmC.

In the studies carried out in the examples on the basis of B. licheniformis DSM 13 and depicted in Examples 1 to 3, said genes exhibited gene inductions which were elevated by at least a factor of 13. Correspondingly preferred embodiments are thus characterized by the first 39 of the genes listed in Table 3.

Further preference is given to those nucleic acid-binding chips of the invention, wherein at least three of the probes are selected from the following 14 genes: yfkN, pstC, yfmQ, pstBA, dhaS homolog (DhaS aldehyde dehydrogenase homolog; SEQ ID No. 17 homolog), gene for a putative phosphatase (SEQ ID No. 61 homolog), phy, cypX, alsS, phoD, pstS, phoB, yvnA, yvmC.

In the studies carried out in the examples on the basis of B. licheniformis DSM 13 and depicted in Examples 1 to 3, said genes exhibited gene inductions which were elevated by at least a factor of 25.

Further preference is given to those nucleic acid-binding chips of the invention, wherein at least three of the probes are selected from the following 8 genes: phy, cypX, alsS, phoD, pstS, phoB, yvnA, yvmC.

In the studies carried out in the examples on the basis of B. licheniformis DSM 13 and depicted in Examples 1 to 3, said genes exhibited gene inductions which were elevated by at least a factor of 40.

Further preference is given to those nucleic acid-binding chips of the invention, wherein at least one, increasingly preferably two or three, of the probes is/are selected from the following 3 genes: phoB, yvnA, yvmC.

In the studies carried out in the examples on the basis of B. licheniformis DSM 13 and illustrated in Examples 1 to 3, these genes exhibited gene inductions which were elevated by at least a factor of 100, in the cases of yvnA and yvmC by more than 115, and in the latter case even by distinctly more than a factor of 140.

In preferred embodiments, nucleic acid-binding chips of the invention are doped with at least 4, 5, 6, 7, 8, 9,10, 11, 12, 13, 14, 15, 16, 17, 18, 19, 20, 22, 24, 26, 28, 30, 35, 40, 45 or 47 of the probes specified for the present invention.

The more said probes respond to a corresponding signal, the more reliable is the information related thereto about the supply or inadequate supply of phosphate at that moment. Thus it is also sensible to combine the particularly meaningful probes which therefore characterize preferred embodiments with apparently less meaningful ones in order to be able to rule out false-positive signals. A further advantage consists of combining, according to the information in Table 2, those probes with one another which produce signals of different strength at different points in time indicated there. Thus it is possible to estimate the time interval from the onset of phosphate deficiency to the sampling and the possibility of said deficiency being attributed to a particular environmental influence—with recording of the culturing conditions.

With respect to their specific sequences, various probes which, however, respond to the same mRNA, for example fragments which hybridize with different regions of the same mRNA (see below), may be present several times in order to increase the read-out reliability, but they are only counted once for the purposes of this aspect of the invention, since they respond, in the best case equally strongly to the same signal and would in principle be exchangeable with one another.

In preferred embodiments of nucleic acid-binding chips of the invention, the total number of all the different probes does with increasing preference not exceed 100, 95, 90, 85, 80, 75, 70, 65, 60, 55 or 50.

This corresponds to the inventive concept stated above, according to which the chips described herein are intended to monitor a special metabolic aspect so that it is not necessary to apply a larger number of probes to the chips in question than is required for recording this situation. This does not affect the situation in which it may be sensible in individual cases to employ more than one probe for detecting the same mRNA and/or to apply individual probes which are related to producing a variable substance of interest. Overall, the present invention is within the specified scope in order to be able to include within the scope of protection chips which for technical reasons have only a few binding sites.

In preferred embodiments of nucleic acid-binding chips of the invention, the probes referred to as being relevant to the invention are those which react to the relevant, or most homologous, in vivo-transcribable genes from the organism chosen for the bioprocess, preferably those which are derived from the relevant, or most homologous, in vivo-transcribable genes of this same organism.

To this end, it has already been stated above that the sequences disclosed in the present application have been obtained from B. licheniformis and should be suitable particularly for monitoring related species, in particular those of the genus Bacillus, owing to the commonly known relationships. This applies both to the identified genes to which it was possible to assign specific functions based on database comparisons and which should in principle encode the same function in other species, and to those which have been defined only by their sequences. To this end, the most closely related genes in the observed organism are used according to the invention for deriving corresponding probes. However, care must be taken here that probes are generated not for pseudo genes but for those which are actually transcribed to mRNA under in vivo conditions, i.e. which result in a nucleic acid signal which can be measured in the cytoplasm.

From a statistical point of view, however, such a chip should be usable more successfully with better interaction of the chosen probes with the nucleic acids to be measured. As a result, especially with a decreasing degree of relationship to B. licheniformis, it becomes increasingly necessary not to rely on the indicated sequences for this hybridization but—if sequence differences exist—to use those for the homologous genes from the species in question. The latter sequences can, as explained above, be obtained by methods per se, in particular gene library screening or PCR with primers based on the sequences disclosed herein (where appropriate in the form of “mismatch primers” containing certain, random sequence variations).

In preferred embodiments of nucleic acid-binding chips of the invention, the organism selected for the bioprocess is a representative of unicellular eukaryotes, Gram-positive or Gram-negative bacteria.

This is because these groups include the commercially most employed organisms, in particular if the bioprocess to be observed is a fermentation. This includes, for example, fermentative processes, for example for producing wine or beer, or biotechnological production of variable substances such as proteins or low molecular-weight compounds.

Various organisms are chosen for a biotechnological method, depending on the type of the desired product. They mean, for the purposes of the invention, not only the producer strains but also any organisms upstream of the production process, for example for the cloning of corresponding genes or the selection of suitable expression vectors. In this connection, the need for recording phosphate limitation is in principle present during each part of the process.

In preferred embodiments of nucleic acid-binding chips of the invention, the unicellular eukaryotes are protozoa or fungi, among these in particular yeast, very particularly Saccharomyces or Schizosaccharomyces.

This is because the latter are employed intensively as host cells, in particular for the gene products of eukaryotes, in addition to being employed for producing alcoholic beverages and other food obtained by fermentation. The former usage is particularly advantageous, if said gene products are to undergo special modifications which can only be carried out by these strains, such as glycosylations of proteins, for example.

This subject matter also includes chips of the invention which are directed to monitoring the course, in particular the growth, of cell cultures of higher eukaryotes, for example rodents or humans. In a certain sense, they may likewise be understood as meaning, at least substantially, unicellular eukaryotes which are of considerable commercial importance, in particular in immunology, for example for producing monoclonal antibodies.

In preferred embodiments of nucleic acid-binding chips of the invention, the Gram-positive bacteria are coryneform bacteria or those of the genera Staphylococcus, corynebacteria or Bacillus, in particular of the species Staphylococcus carnosus, Corynebacterium glutamicum, Bacillus subtilis, B. licheniformis, B. amloliquefaciens, B. agaradherens, B. stearothermophilus, B. globigii or B. lentus, and very particularly B. licheniformis.

This is because they are industrially particularly important producer strains. They are employed in particular for producing low molecular-weight chemical compounds, for example vitamins or antibiotics, or for producing proteins, in particular enzymes. Particular mention must be made here of amylases, cellulases, lipases, oxidoreductases and proteases. The particular orientation toward B. licheniforms can be explained by the fact that the sequences indicated in the sequence listing have been obtained from this species and that, as described in Examples 2 and 3, it was possible to prove their connection with the transition to phosphate deficiency.

In no less preferred embodiments of nucleic acid-binding chips of the invention, the Gram-negative bacteria are those of the genera E. coli and Klebsiella, in particular derivatives of Escherichia coli K1 2, of Escherichia coli B or Klebsiella planticola, and very particularly derivatives of the strains Escherichia coli BL21 (DE3), E. coli RV308, E. coli DH5α, E. coli JM109, E. coli XL-1 or Klebsiella planticola (Rf).

This is because they are used for producing biological valuable substances, both on the laboratory scale, for example cloning and expression analysis, and on the industrial scale.

In preferred embodiments of nucleic acid-binding acids of the invention, at least one, increasingly preferably a plurality, of the probes specified in connection with the invention described herein is/are derived from the sequences listed in the sequence listing under numbers SEQ ID No. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91 and 93.

This is because it was possible to prove the connection of these genes with the transition to phosphate deficiency in B. licheniformis, as described in Examples 2 and 3. These sequences should therefore be used in particular, if B. licheniformis or other, especially related, Bacillus species are to be monitored.

Preferred embodiments of nucleic acid-binding chips of the invention are those which are additionally doped with at least one probe for an additional gene, in particular one which is metabolically associated with the gene(s) additionally expressed depending on the process, very particularly for one of these or this one itself.

As explained above, the observed processes serve an industrial interest which is often related to further specific genes. This is, for example if a protein is to be produced, the gene for this protein and, if a low molecular-weight compound is to be produced, one or more gene products which are on the synthetic pathway of the compound in question or regulate said pathway. Other genes intrinsic to the cell may also be affected, for example metabolic genes which must be increasingly produced in the course of product production, for example an oxidoreductase intrinsic to the cell, if the product is to be obtained from a reactant or an intermediate by oxidation or reduction.

Moreover, particularly biological processes, in particular production of commercially relevant compounds by microorganisms, usually employ strains directed to the process in question rather than wild type strains. This includes, apart from transformation with the genes responsible for actual production of the product, provision with selection markers or further metabolic adjustments up to auxotrophies. Such strains have a particular profile of demands on the growth conditions, and some of them have metabolic genes which have been mutated compared with the wild type genes. Since chips of the invention are advantageously intended to be directed to exactly these strains, very particularly the observed bioprocess, these strain-specific peculiarities should be taken into account and may be reflected in the choice of the probes in question.

In preferred embodiments of such nucleic acid-binding chips of the invention, the gene additionally expressed depending on the process is that for a commercially usable protein, in particular an amylase, cellulose, lipase, oxidoreductase, a hemicellulase or protease, or one which is on a synthetic pathway of a low molecular-weight chemical compound or which at least partially regulates said pathway.

These are then directed particularly to those bioprocesses, especially fermentations, in which said proteins are produced. The latter are commercially particularly important enzymes which are used, for example, in the food industry or detergent industry. In the latter case in particular for removing soilings which are hydrolysable by amylases, cellulases, lipases, hemicellulases and/or proteases, for the treatment of the respective materials, in particular by cellulases, or for providing an enzymic bleaching system based on an oxidoreductase.

The latter variant falls within the area of biotransformation, according to which particular, where appropriate additionally introduced, metabolic activities of microorganisms are utilized for the synthesis of chemical compounds.

In preferred embodiments of nucleic acid-binding chips of the invention, one, preferably a plurality, of the probes specified in connection with the invention described herein is/are provided in single-stranded form, in the form of the codogenic strand.

This embodiment has the aim of improving hybridization between the probe and the sample to be detected. This applies in particular to the case in which the relevant mRNA content is actually determined from the sample. Since said mRNA is single-stranded and its sequence corresponds to the coding strand of the DNA, optimal hybridization with the complementary, i.e. codogenic, strand should ensue.

In preferred embodiments of nucleic acid-binding chips of the invention, one, preferably a plurality, of the probes specified in connection with the invention described herein is/are provided in single-stranded form, in the form of the codogenic strand.

This embodiment has the aim of improving hybridization between the probe and the sample to be detected. This applies in particular to the case in which the relevant mRNA content is actually determined from the sample. Since said mRNA is single-stranded and its sequence corresponds to the coding strand of the DNA, optimal hybridization with the complementary, i.e. codogenic, strand should ensue.

In preferred embodiments of nucleic acid-binding chips of the invention, one, preferably a plurality, of the probes referred to as being relevant to the invention is/are provided in the form of a DNA or a nucleic acid analog, preferably a nucleic acid analog.

This embodiment has the aim of improving the durability and multiple usability of the chips of the invention. This need arises in particular during a single observed process in the course of which constant monitoring is desirable. The durability of chips of the invention, in particular toward nucleic acid-hydrolyzing enzymes, is already increased by providing the probes in the form of a DNA, since the latter is hydrolytically less sensitive per se than an RNA, for example. Even more stable are nucleic acid analogs in which, for example, the phosphate of the sugar-phosphate backbone has been replaced with a chemically different building block which cannot be hydrolyzed, for example, by natural nucleases. Such compounds are known in principle in the prior art and are, if desired, commercially synthesized by companies specializing in this for desired sequences to be indicated in each case. The probes in question can be synthesized, for example, according to the template of the sequences indicated in the sequence listing.

In preferred embodiments of nucleic acid-binding chips of the invention, one, preferably a plurality of the probes referred to as being relevant to the invention comprises/comprise gene regions which are transcribed into mRNA by the organism to be studied, in particular the gene regions close to the 5′ end of said mRNA.

This takes into account the aspect that in many cases the regulatory DNA sections are also assigned to a special gene. However, the chip of the invention is actually intended to be employed for detecting the mRNA actually present in the observed cells, so that, for the purpose contemplated herein, only the gene section which is actually translated into mRNA is important. Secondly, it must also be considered that introns occur, in particular in eukaryotes, i.e. the coding region is interrupted by sections which are not translated into mRNA. Probes containing introns should therefore respond to the mRNA in question only poorly or not at all. To implement this aspect, it is advisable to use cDNA sequences rather than genomic DNA sequences, i.e. those sequences which have been obtained on the basis of the actual mRNA.

Furthermore, detection of an mRNA often does not require hybridization over the entire length of the sequence. The specific probes therefore need to comprise usually only a smaller of the gene transcribed to mRNA. Advantageous here is a selection of a region close to the 5′ end of the mRNA, since this region is the first to be transcribed to mRNA and therefore the first to be detectable after activation of the gene. This benefits on-line detection.

In preferred embodiments of nucleic acid-binding chips of the invention, one, preferably a plurality, of the probes referred to as being relevant to the invention reacts/react to fragments of the relevant nucleic acids, in particular to those whose respective mRNA has a low degree of secondary folding, based on the particular total mRNA.

This is another aspect in order to optimize hybridization between the probes and the mRNA to be detected. This is because mRNA molecules frequently have a secondary structure which is based on hybridization of individual mRNA regions with intrinsic, other regions. Thus, for example loop or stem-loop structures are formed. Such regions, however, usually hybridize less readily with other nucleic acid molecules, even if the latter are homologous. Regions of this kind can be calculated quite accurately by computer programs directed thereto (see below). Thus, to implement this aspect, the gene whose activity is desired to be determined for an organism of interest should be analyzed by such a program and, in order to obtain a suitable probe—usually comprising only a subsection (see below)—, sections should be used for which only a low degree of mRNA secondary structures is predicted.

In preferred embodiments of nucleic acid-binding chips of the invention, one, preferably a plurality, of the probes referred to as being relevant to the invention has/have a length of increasingly preferably less than 200, 150, 125 or 100 nucleotides, preferably from 20 to 60 nucleotides, particularly preferably from 45 to 55 nucleotides.

This is because the probes used for the detection reaction need to comprise only part of the mRNA to be detected, as long as the signal obtainable via them is still specific enough. This specificity, the distinguishability of different mRNAs, sets the lower limit of the length of the probes in question and must, where appropriate, be determined in preliminary experiments.

The identification of suitable probe lengths and probe regions is known per se to the skilled worker and is normally carried out with the aid of specialized software. Examples of such software are the programs Array Designer from Premier Biosoft International, USA, and Vector NTI® Suite, V. 7, obtainable from InforMax, Inc., Bethesda, USA. These software programs take into account, for example, also predefined probe lengths and melting temperatures, in addition to the secondary structures already mentioned.

In preferred embodiments of nucleic acid-binding chips of the invention, binding of the mRNA to the relevant probe referred to as being relevant to the invention triggers an electric signal.

The article by J. Wang (Acc. Chem. Res.; ISSN 0001-4842; Rec. Sept. 12, 2001, pp. A-F), mentioned above, discusses the advantages of an electrically analyzable system over an optical system. It also refers to various embodiments of such sensors, which have been developed in the prior art.

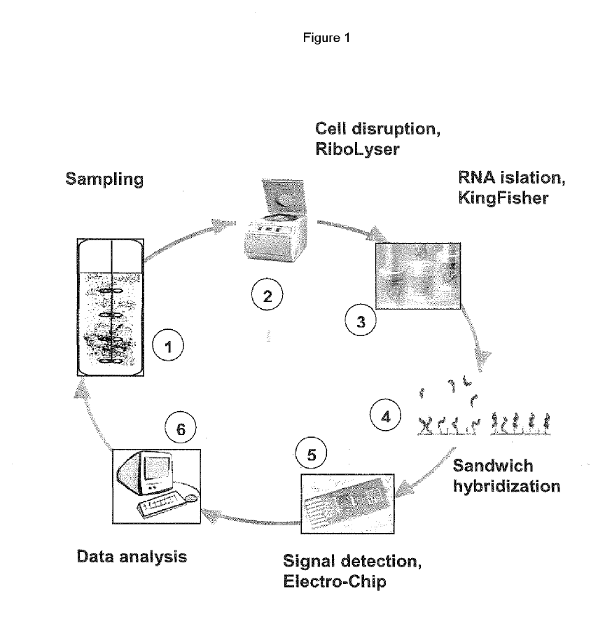

Thus the time interval from sampling to measuring of the signal is currently approximately 24 h for optically analyzable chips. With the aid of an electrical system, the required time is currently less than 2 h (cf. FIG. 1). In contrast to this, the number of simultaneously analyzable samples is currently in the two-digit range with electrically analyzable chips, but rapid development gives reason to believe that this order of magnitude may be exceeded soon. Limiting in this are the electronic evaluation units for the various signals.

An example of a method of quantifying mRNA, which is established in the prior art, is RT-PCT. This method is described in the article “Quantification of Bacterial mRNA by One-Step RT-PCR Using the LightCycler System” (2003) by S. Tobisch, T. Koburger, B. Jürgen, S. Leja, M. Hecker and T. Schweder in BIOCHEMICA, Volume 3, pages 5 to 8. In comparison with this, detection via electrochips has another advantage, namely higher reliability of the data, since these have a distinctly smaller range of fluctuation compared to RT-PCR.

The preparation of corresponding electronically analyzable chips is described, for example, in the patent applications WO 00/62048 A2, WO 00/67026 A1 and WO 02/41992, whose entire disclosure is incorporated into the present application.

The way in which electrically readable chips of a particularly preferred embodiment function can be described as follows: the gene-specific probes are bound covalently in a manner known per se to magnetic beads located in specifically designed chambers of said chips. The corresponding mRNA specifically hybridizes to the particular beads in this hybridization chamber whose temperature can be controlled and which can be flushed by the solutions in question. The beads are retained in this chamber by a magnet. After hybridization of the RNA samples to the beads-bound DNA probes, unbound RNA is removed in a washing step, as a result of which only specific hybrids are still present in the incubation chamber, bound to the magnetic beads.

After washing, a detection probe labeled by a biotin-extravidin-bound alkaline phosphatase is introduced into the incubation chamber. This probe binds to a second free region of the hybridized mRNA. This hybrid is then washed again and incubated with the alkaline phosphatase substrate, para-aminophenol phosphate (pAPP). The enzymic reaction in the incubation chamber releases the redox-active product, para-aminophenol (pAP). The latter is then passed over the Red/Ox electrode on the electrical chip and the signal is transmitted to a potentiostat.

A system-specific software (for example MCDDE32) reads the data obtained, and the results may be evaluated and displayed on a computer with the aid of a further program (for example Origin).

This process can of course be varied with regard to both the technical design of the chips and evaluation. Thus, for example, the detection reaction may be carried out by way of a different reaction, but preferably a redox reaction due to the electrical principle of measurement.

One achievement of the present invention is to have identified phosphate metabolism-specific and thus process-critical genes and to have made them accessible to analysis via correspondingly designed biochips. The advantage of chips over conventional detection methods comprises, in addition to the time saved and higher accuracy, the possibility of detecting the activities of a plurality of different genes in the same sample at the same time by providing a plurality of probes on a single support and of said activities resulting in a more solid and more detailed picture, for example with regard to the time at which a phosphate deficiency has started, with the application to a special problem, described herein.

A separate subject matter of the invention is therefore the simultaneous use of nucleic acid probes or nucleic acid analog probes for at least three of the following 47 genes: yhcR, tatCD, ctaC, gene for a putative acetoin reductase (SEQ ID No. 53 homolog), spoIIGA, nasE, pstA, spoIIAA, gene for a hypothetical protein (SEQ ID No. 65 homolog), yhbD, cotE, gene for a conserved hypothetical protein (SEQ ID No. 59 homolog), yurl, spoVID, gene for a putative aromatic-specific dioxygenase (SEQ ID No. 55 homolog), yhbE, gene for a putative benzoate transport protein (SEQ ID No. 49 homolog), pstBB, spoIIIAH, gene for a hypothetical protein (SEQ ID No. 63 homolog), spoIIQ, spoIIIAG, yvmA, gene for a putative ribonuclease (SEQ ID No. 93 homolog), dhaS, yrbE, gene for a putative decarboxylase/dehydratase (SEQ ID No. 57 homolog), htpG, yfkH, spoIIAB, spoIIIAF, alsD, gdh, yfkN, pstC, yfmQ, pstBA, dhaS homolog (DhaS aldehyde dehydrogenase homolog; SEQ ID No. 17 homolog), gene for a putative phosphatase (SEQ ID No. 61 homolog), phy, cypX, alsS, phoD, pstS, phoB, yvnA, yvmC, bound to a nucleic acid-binding chip, preferably to the same chip, for determining the physiological state of an organism undergoing a biological process.

As explained above, these 47 genes are selected so as to deliver a picture of the situation of the phosphate metabolism of the observed organism, since they are, as described in Examples 1 to 3, significantly and comparatively specifically induced during transition of the Gram-positive bacterium B. licheniformis to a phosphate deficiency condition. A comparable statement can also be expected for other organisms which have the homologous genes or proteins with essentially the same metabolically relevant properties.

As likewise described in detail above, such gene activities may in principle be determined in various ways, for example by Northern hybridization.

However, analysis with the aid of a nucleic acid-binding chip, in particular an above-described chip, enables a plurality of gene activities to be determined very efficiently at the same time and, moreover, very much on-line. As a result, the metabolic alterations of an organism undergoing a biological process may be observed at line and, where appropriate, intervened in in a regulatory manner.

The above comments on nucleic acid-binding chips apply to the uses specified herein of the probes in question accordingly.

Accordingly, preference is given here to those uses, wherein the total number of all the different phosphate metabolism-specific probes does not exceed 100.

Increasing preference is given to the total number of all the different phosphate metabolism-specific probes not exceeding 95, 90, 85, 80, 75, 70, 65, 60, 55 or 50, again including positive controls from the remaining phosphate metabolism.

Accordingly, further preference is given here to those uses, wherein the probes indicated above as inventive are employed with increasing preference in the order indicated above as preferable (inverse to the order in Table 3).

According to the above comments, increasing preference is given to the following of the uses specified above of nucleic acid probes or nucleic acid analog probes:
the use, wherein at least three of the nucleic acid probes or nucleic acid analog probes is/are specific for genes from the following 39 genes: gene for a hypothetical protein (SEQ ID No. 65 homolog), yhbD, cotE, gene for a conserved hypothetical protein (SEQ ID No. 59 homolog), yurl, spoVID, gene for a putative aromatic-specific dioxygenase (SEQ ID No. 55 homolog), yhbE, gene for a putative benzoate transport protein (SEQ ID No. 49 homolog), pstBB, spoIIIAH, gene for a hypothetical protein (SEQ ID No. 63 homolog), spoIIQ, spoIIIAG, yvmA, gene for a putative ribonuclease (SEQ ID No. 93 homolog), dhaS, yrbE, gene for a putative decarboxylase/dehydratase (SEQ ID No. 57 homolog), htpG, yfkH, spoIIAB, spoIIIAF, alsD, gdh, yfkN, pstC, yfmQ, pstBA, dhaS homolog (DhaS aldehyde dehydrogenase homolog; SEQ ID No. 17 homolog), gene for a putative phosphatase (SEQ ID No. 61 homolog), phy, cypX, alsS, phoD, pstS, phoB, yvnA, yvmC;
among these preferably for at least three of the following 14 genes: yfkN, pstC, yfmQ, pstBA, dhaS homolog (DhaS aldehyde dehydrogenase homolog; SEQ ID No. 17 homolog), gene for a putative phosphatase (SEQ ID No. 61 homolog), phy, cypX, alsS, phoD, pstS, phoB, yvnA, yvmC;
among these particularly preferably for at least three of the following 8 genes: phy, cypX, alsS, phoD, pstS, phoB, yvnA, yvmC; and
among these very particularly preferably for at least one of the following 3 genes: phoB, yvnA, yvmC.

According to the above, the uses of the invention are preferably those of at least 4, 5, 6, 7, 8, 9, 10, 11, 12, 13, 14, 15, 16, 17, 18, 19, 20, 22, 24, 26, 28, 30, 35, 40, 45 or 47 of the specified probes at the same time.

According to the above, preferred uses of the invention are those, wherein the total number of all the different probes does, with increasing preference, not exceed 100, 95, 90, 80, 75, 70, 65, 60, 55, 50, 40, 30, 20 or 10.

According to the above, further preference is given to uses of the invention for determining a change in the phosphate metabolism of the organism undergoing the biological process, preferably for detecting a phosphate deficiency condition.

According to the above, further preference is given to uses of the invention, wherein at least one, increasingly preferably a plurality, of the specified probes is/are derived from the sequences listed in the sequence listing under the numbers SEQ ID No. 1, 3, 5, 7, 9, 11, 13, 15, 17, 19, 21, 23, 25, 27, 29, 31, 33, 35, 37, 39, 41, 43, 45, 47, 49, 51, 53, 55, 57,59, 61, 63, 65, 67, 69, 71, 73, 75, 77, 79, 81, 83, 85, 87, 89, 91 and 93.

A separate subject matter of the invention are methods of determining the physiological state of an organism under a biological process by using a nucleic acid-binding chip of the invention.

These methods in principle comprise removing samples of the observed organism during said process, without interrupting the latter, and isolating the mRNA from said samples. These compounds or, where appropriate, compounds derived therefrom, such as cDNA, for example, are passed across an above-described nucleic acid-binding chip which is treated taking into account the method steps indicated above—such as, for example, adequate incubation time or washing off nonspecifically binding nucleic acids and is finally delivered to the detection apparatus. A method protocol of this kind is depicted in principle in FIG. 1 on the basis of the example of electrically analyzable chips.

The above comments on nucleic acid-binding chips apply accordingly to the methods specified herein of determining the physiological state of an organism undergoing a biological process.

Preference is given to methods of the invention, wherein a change in the phosphate metabolism of the organism undergoing the biological process, preferably a phosphate deficiency condition, is determined.

With respect to this field of use, the genes described in the examples and listed above have been selected. Their significant induction, at least in B. licheniformis, is accompanied by the onset of a phosphate deficiency condition, resulting in the ability of said methods to detect said genes particularly reliably, and not only in B. licheniformis but, with increasingly improving prospects of success, also in increasingly related species (see above). Moreover, this metabolic situation represents a critical point in the lifecycle of many microorganisms. Thus, as illustrated in the examples, the particular onset of phosphate deficiency has always also been associated with a transition to the stationary growth phase. Methods which aid the early recognition of this point serve to delay said transition and, in particular with industrially utilized fermentations, prolong the phase of production of a valuable substance.

According to the above, preference is given to those methods of the invention, wherein the organism selected for the bioprocess is a representative of unicellular eukaryotes, Gram-positive or Gram-negative bacteria.

According to the above, preference is given among said methods to those methods of the invention, wherein the unicellular eukaryotes are protozoa or fungi, among these in particular yeast, very particularly Saccharomyces or Schizosaccharomyces.

According to the above, preference is given here also to those methods of the invention, wherein the Gram-positive bacteria are coryneform bacteria or those of the genera Staphylococcus, corynebacteria and Bacillus, in particular of the species Staphylococcus carnosus, Corynebacterium glutamicum, Bacillus subtilis, B. licheniformis, B. amloliquefaciens, B. agaradherens, B. stearothermophilus, B. globigii or B. lentus, and very particularly B. licheniformis.

According to the above, no less preference is given here also to those methods of the invention, wherein the Gram-negative bacteria are those of the genera E. coli and Klebsiella, in particular derivatives of Escherichia coli K12, of Escherichia coli B or Klebsiellia planticola, and very particularly derivatives of the strains Escherichia coli BL21 (DE3), E. coli RV308, E. coli DH5α, E. coli JM109, E. coli XL-1 or Klebsiella planticola (Rf).

According to the above, preference is given to those methods of the invention, wherein those specified probes are used, which are derived from the SEQ ID Nos. 1, 3, 5, 7, 9, 11, 15, 19, 23, 25, 29, 31, 33, 37, 43, 45, 49, 51, 53, 55, 59, 61, 65, 67, 69, 71, 73, 75, 77, 81, 83, 85, 87, 89, 91 or 93 indicated in the sequence listing.

Among these, preference is again given to those methods which employ the probes specified herein for the above-listed Gram-positive bacteria, in particular B. licheniformis, since these sequences have been isolated from this very organism and can therefore be applied most successfully to this species.

According to the above, preference is given to those methods of the invention, wherein the physiological state is determined at various points in time of the same process, preferably using a plurality of structurally identical nucleic acid-binding chips, particularly preferably of the same nucleic acid-binding chip.

According to the above, preference is furthermore given to those methods of the invention, wherein the process is a fermentation, in particular the fermentative production of a commercially usable product, particularly preferably the production of a protein or of a low molecular-weight chemical compound.

According to the above, preference is given among these methods to those methods of the invention, wherein the low molecular-weight chemical compound is a natural substance, a food supplement or a pharmaceutically relevant compound.

According to the above, preference is alternatively also given to those methods of the invention, wherein the protein is an enzyme, in particular one of the group of α-amylases, proteases, cellulases, lipases, oxidoreductases, peroxidases, laccases, oxidases and hemicellulases.

A separate subject matter of the invention is also the possible uses of nucleic acid-binding chips of the invention, as have been described in detail above, for determining the physiological state of an organism undergoing a biological process.

The above comments on nucleic acid-binding chips apply accordingly to the uses specified herein of determining the physiological state of an organism undergoing a biological process.

According to the above, preference is given to those uses of the invention, wherein a change in the phosphate metabolism of the organism undergoing the biological process, preferably a phosphate deficiency condition, is determined.

According to the above, preference is furthermore given to those uses of the invention, wherein the organism selected for the bioprocess is a representative of unicellular eukaryotes, Gram-positive or Gram-negative bacteria.

According to the above, preference is given among said uses to those uses of the invention, wherein the unicellular eukaryotes are protozoa or fungi, among these in particular yeast, very particularly Saccharomyces or Schizosaccharomyces.

According to the above, no less preference is given here also to those uses of the invention, wherein the Gram-positive bacteria are coryneform bacteria or those of the genera Staphylococcus, corynebacteria and Bacillus, in particular of the species Staphylococcus carnosus, Corynebacterium glutamicum, Bacillus subtilis, B. licheniformis, B. amloliquefaciens, B. agaradherens, B. stearothermophilus, B. globigii or B. lentus, and very particularly B. licheniformis.

According to the above, no less preference is given here also to those uses of the invention, wherein the Gram-negative bacteria are those of the genera E. coli and Klebsiella, in particular derivatives of Escherichia coli K12, of Escherichia coli B or Klebsiellia planticola, and very particularly derivatives of the strains Escherichia coli BL21 (DE3), E. coli RV308, E. coli DH5α, E. coli JM109, E. coli XL-1 or Klebsiella planticola (Rf).

According to the above, preference is given to those uses of the invention, wherein those specified probes are used which have been derived from the SEQ ID Nos. 1, 3, 5, 7, 9, 11, 15, 19, 23, 25, 29, 31, 33, 37, 43, 45, 49, 51, 53, 55, 59, 61, 65, 67, 69, 71, 73, 75, 77, 81, 83, 85, 87, 89, 91 or 93 indicated in the sequence listing.

For the reason specified above, among these uses, preference is given in turn to those uses which employ the probes specified herein for the above-listed Gram-positive bacteria, in particular B. licheniformis.

According to the above, preference is given to those uses of the invention, wherein the physiological state is determined at various points in time of the same process, preferably using a plurality of structurally identical nucleic acid-binding chips, particularly preferably of the same nucleic acid-binding chip.

According to the above, preference is furthermore given to those uses of the invention, wherein the process is a fermentation, in particular the fermentative production of a commercially usable product, particularly preferably the production of a protein or of a low molecular-weight chemical compound.

According to the above, preference is given among these methods to those uses of the invention, wherein the low molecular-weight chemical compound is a natural substance, a food supplement or a pharmaceutically relevant compound.

According to the above, preference is alternatively also given to those uses of the invention, wherein the protein is an enzyme, in particular one of the group of α-amylases, proteases, cellulases, lipases, oxidoreductases, peroxidases, laccases, oxidases and hemicellulases.

The present invention is additionally illustrated by the examples below.

### EXAMPLES

All molecular-biological work is carried out by standard methods as can be found, for example, in the manual by Fritsch, Sambrook and Maniatis “Molecular cloning: a laboratory manual”, Cold Spring Harbour Laboratory Press, New York, 1989, or comparable specialist literature. Enzymes and kits are used according to the instructions of the particular manufacturers.

### Example 1

Identification of the Gene Probes by Chip Analyses

Culturing of Bacteria and Isolation of Samples

Cells of the Bacillus licheniformis DSM13 strain (obtainable from Deutsche Sammlung von Mikroorganismen und Zellkulturen GmbH, Mascheroder Weg 1b, 38124 Braunschweig, Germany; http://www.dsmz.de) were cultured in phosphate-limited, synthetic Belitsky minimal medium (0.28 mM final concentration) with constant shaking at 270 rpm and 37° C. Said medium has the following composition: (1.) basic medium (pH 7.5): 0.015 M (NH₄)₂SO₄, 0.008 M MgSO₄×7H₂O, 0.027 M KCl, 0.007 M sodium citrate×2H₂O, 0.050 M Tris-HCl and 0.009 M glutamic acid; (2.) supplements: 0.2 M KH₂PO₄, 0.039 M L-tryptophan-HCl, 1 M CaCl₂×2H₂O, 0.0005 M FeSO₄×7H₂O, 0.025 M MnSO₄×4H₂O and 20% (w/v) glucose; and as (3.) medium supplementing plan: 0.14 ml of KH₂PO₄, 0.2 ml of CaCl₂, 0.2 ml of FeSO₄, 0.04 ml of MnSO₄, 1 ml of glucose. All values refer to 100 ml of basic medium.

In the course of establishing the growth profile, the control sample was removed at an optical density at 500 nm (OD₅₀₀) of from 0.4 to 0.5, with another sample (“transition”) being removed in the transient growth phase at OD₅₀₀ 0.8 to 0.9. The supplementing plan for the Belitzky minimal medium, indicated above, has been set up in such a way that phosphate deficiency starts at OD₅₀₀ 0.8 to 1.0, thereby introducing the stationary phase. Further samples were taken after another 30, 60 and 120 min. Immediately after their removal, an equal volume of Killing buffer (20 mM NaN₃; 20 mM Tris-HCl, pH 7.5; 5 mM MgCl₂) cooled to 0° C. was added to the samples. Immediately thereafter, the cell pellet was removed by centrifugation at 4° C. and 15000 rpm for 5 min, the supernatant was discarded, and the pellet was either frozen at −80° C. or worked up further immediately.

Cell Disruption

The cells were disrupted using the “Hybaid RiboLyser™ Cell Disruptor” (Thermo Electron Corporation, Dreieich, Germany). This method is based on mechanically destroying the cell wall and the cell membrane with the aid of glass beads of approx. 0.1 mm in size (Sartorius BBI, Melsungen, Germany). The cells to be disrupted which had been resuspended in lysis buffer II (3 mM EDTA; 200 mM NaCl) beforehand were introduced together with the glass beads into a glass (strain maintenance) tube. Acidic phenol was also added in order to prevent RNA degradation by RNases. Said tube was then put inside the RiboLyser, wherein the glass beads colloided with the cells with vigorous shaking, thereby causing disruption of the cells.

Isolation of Total RNA

After cell disruption, the RNA-containing aqueous phase is separated by centrifugation from the protein and cell fragments, chromosomal DNA and the glass beads, and the RNA is isolated therefrom with the aid of the KingFisher mL apparatus (Thermo Electron Corporation, Dreieich, Germany) using the MagNA Pure LC RNA Isolation Kit I (Roche Diagnostics, Penzberg, Germany). This purification is based on the RNA binding to magnetic glass particles in the presence of chaotropic salts which also inactivate RNases. The magnetic particles here serve as a means of transporting the RNA between various reaction vessels filled with binding, washing and elution buffers. The KingFisher mL utilized for this is a kind of pipetting robot which transports the particles with the bound RNA back and forth between the vessels with the aid of magnets and is also utilized for mixing the samples. Finally, the RNA is detached from the magnetic particles and is then present in a purified state.

RNA Quality and Quantity Control

The purification of RNA is followed by quality and quantity control. The Agilent Bioanalyzer 2100 apparatus (Agilent Technologies, Berlin, Germany) utilized for this enables the analysis of RNA on lab-on-a-chip scale. Together with the Agilent “RNA 6000 Nano Kit” total RNA is fractionated gel electrophoretically and thereby offers the possibility of examining the quality with regard to partial degradation and contaminations. This involves detecting ribosomal RNA (16 S and 23 S rRNA). If the latter appear as clear bands, the RNA can be assumed not to have been degraded during processing and therefore to be intact and to be able to be introduced to the subsequent examinations. In addition, the exact concentration is also determined here.

Transcriptome Analyses

Transcriptome analyses were carried out using total genomic B. licheniformis DSM13 DNA microarrays which had been prepared in the usual manner (for example according to WO 95/11995 A1) and which can be analyzed by an optical system. These DNA microarrays contained two copies of virtually each B. licheniformis gene, so that it was possible to analyze two samples in parallel on the same chip and to average the values obtained.

The principle of the measurement carried out consists of transcribing in vitro the particular mRNA molecules from the sample taken via reverse transcription to RNA, with one of the added deoxyribonucleotides carrying a dye marker. These labeled molecules are then hybridized with the known probes located at known sites of the chip, and the particular strength of the signal at the sites in question, which can be attributed to the fluorescent marker, is optically recorded. When using two different fluorescent markers for a control and for a sample actually to be studied, which are made to hybridize simultaneously and thereby compete for binding to the probe presented, different color values are obtained which provide a measure of the ratio of the concentration of the control to that of the sample.

dUTP labeled with the fluorescent dye cyanine 3 or cyanine 5 was chosen for this labeling. Thus, in each case 25 μg of total RNA of the control (OD₅₀₀ 0.4) were labeled with the fluorescent dye cyanine 3 (Amersham Biosciences Europe GmbH, Freiburg, Germany) and 25 μg of total RNA of the particular stress sample (transient phase, 30, 60 and 120 min) were labeled with the fluorescent dye cyanine 5 (GE Healthcare, Freiburg, Germany). Competitive hybridization of the two samples was carried out at 42° C. on the conventional, total genomic B. licheniformis DSM13 DNA microarray for at least 16 hours.

After washing of the array for removing unspecific bonds, the array was read out optically with the aid of the ScanArray® Express Laser Scanner (PerkinElmer Life and Analytical Sciences, Rodgau-Jügesheim, Germany). All hybridizations were repeated, with the samples being labeled with the in each case other dye (dye swap method). Quantitative evaluation of the arrays was carried out using the ScanArray® Express software (available from PerkinElmer Life and Analytical Sciences, Rodgau-Jügesheim, Germany) according to the manufacturer's instructions and with standard parameters.

The arrays were normalized and evaluated with the aid of the Lucidea Score Card controls (GE Healthcare, Freiburg, Germany). With the aid of this “Score Card” which is used for controlling hybridization efficiency and quality, known control DNAs and “spikes” in the form of oligos have been applied to the array according to the manufacturer's instructions and admixed with complementary sequences during hybridization. Thus it was possible to control, after scanning, the success of said hybridization and incorporation of the dyes. The controls should be present in the same amounts in both samples and therefore appear yellow after scanning or have a ratio of 1 between both channels. The spikes are specific for the particular sample and are applied in various dilutions, i.e. they appear red or green after scanning for the particular sample.

For expression of the genes, averages of the two hybridizations and the particular standard deviations were calculated. For a significant induction or significant repression, the following thresholds of the ratio of the amount of RNA of the particular gene to the control value were observed: genes whose RNA has a ratio of >3 (i.e. at least a three-fold increase) are regarded as induced; genes having an RNA ratio of <0.3 (i.e. a decrease to less than 30%) are distinctly repressed. These results are listed in Example 2.

### Example 2

Genes Induced Under Phosphate Deficiency

Table 1 below lists all 235 Bacillus licheniformis DSM13 genes determined in Example 1 whose induction (of at least a factor of 3) was observed under the conditions of phosphate deficiency described in Example 1. The first two columns indicate the particular name of the derived protein and, respectively, its abbreviation (if available); the “Bli number” corresponds to the “locus_tag” of the B. licheniformis complete genome accessible under the entry AE017333 (bases 1 to 4 222 645) in the GenBank database (National Center for Biotechnology Information NCBI, National Institutes of Health, Bethesda, Md., USA; http://www.ncbi.nlm.nih.gov; as of 12.2.2004); this is followed by the factors of increasing the concentration of the in each case corresponding mRNAs, observed at the times indicated at the top.

TABLE 1
The 235 Bacillus licheniformis DSM13 genes determined in
Example 1, whose induction (of at least a factor of 3) under
phosphate deficiency was observed (explanations: see text).

| Gene name/gene function | ID | Bli-No. | Transition | 0.5 h | 1 h | 2 h |
|---|---|---|---|---|---|---|
| class III stress response- | ClpC | BLi00104 | 3.37 | 3.12 | 3.07 | 4.09 |
| related ATPase | | | | | | |
| family serine protease, | ClpP | BLi03615 | 4.13 | 4.12 | 3.27 | 4.26 |
| possible phage related | | | | | | |
| general stress protein | Ctc | BLi00065 | 3.37 | 3.38 | 3.29 | 2.30 |
| class III heat-shock protein | HtpG | BLi04256 | 19.93 | 4.26 | 3.42 | 4.35 |
| serine protease Do (heat- | HtrA | BLi01390 | 3.59 | 3.14 | 4.71 | 3.13 |
| shock protein) | | | | | | |
| modulator of CtsR repression | McsB | BLi00103 | 3.09 | 3.00 | 3.07 | 1.98 |
| similar to general stress | YtxG | BLi03130 | 3.42 | 2.85 | 2.60 | 1.60 |
| protein | | | | | | |
| similar to HtrA-like serine | YvtA | BLi03481 | 3.41 | 3.77 | 3.63 | 4.41 |
| protease | | | | | | |
| similar to capsular | YwqC | BLi03855 | 3.68 | 3.79 | 8.19 | 7.06 |
| polysaccharide biosynthesis | | | | | | |
| similar to capsular | YwqE | BLi03854 | 2.57 | 3.46 | 4.66 | 4.16 |
| polysaccharide biosynthesis | | | | | | |
| similar to capsular | YwsC | BLi03839 | 6.03 | 3.37 | 4.61 | 3.25 |
| polyglutamate biosynthesis | | | | | | |
| penicillin-binding protein | DacF | BLi02498 | 3.25 | 3.24 | 3.56 | 5.10 |
| (putative D-alanyl-D-alanine | | | | | | |
| carboxypeptidase) | | | | | | |
| cell wall hydrolase (major | LytE | BLi01008 | 3.55 | 3.31 | 3.51 | 2.63 |
| autolysin) | | | | | | |
| biosynthesis of teichuronic acid | TuaA | BLi03807 | 2.12 | 3.40 | 1.81 | 2.1 |
| beta-lactamase precursor | PenP | BLi00280 | 3.73 | 3.29 | 4.11 | 5.13 |
| (penicillinase) | | | | | | |
| aminoglycoside 6- | AadK | BLi00217 | 3.33 | 3.85 | 2.48 | 2.76 |
| adenylyltransferase | | | | | | |
| cytochrome P450-like enzyme | CypA | BLi02822 | 1.17 | 0.95 | 2.98 | 3.08 |
| cytochrome P450-like enzyme | CypX | BLi03567 | 15.40 | 1.65 | 37.31 | 41.21 |
| peptidyl methionine sulfoxide | MsrA | BLi02303 | 3.41 | 2.05 | 3.07 | 4.01 |
| reductase | | | | | | |
| superoxide dismutase | SodA | BLi02679 | 4.04 | 3.72 | 3.45 | 2.53 |
| similar to macrolide | YjiC | BLi01948 | 4.02 | 3.01 | 2.12 | 3.11 |
| glycosyltransferase | | | | | | |
| similar to immunity to | YkfA | BLi01397 | 1.21 | 1.52 | 3.36 | 3.62 |
| bacteriotoxins | | | | | | |
| similar to single-strand DNA- | YwpH | BLi03869 | 3.18 | 1.36 | 5.27 | 4.94 |
| binding protein | | | | | | |
| nuclease inhibitor | DinB | BLi02244 | 4.67 | 5.19 | 3.25 | 3.30 |
| putative ribonuclease | | BLi03719 | 7.98 | 11.03 | 13.68 | 19.00 |
| cytochrome caa3 oxidase | CtaA | BLi01704 | 0.83 | 1.27 | 3.45 | 5.28 |
| (required for biosynthesis) | | | | | | |
| cytochrome caa3 oxidase | CtaB | BLi01705 | 0.95 | 1.26 | 2.89 | 3.22 |
| (assembly factor) | | | | | | |
| cytochrome caa3 oxidase | CtaC | BLi01706 | 1.05 | 1.29 | 7.62 | 10.87 |
| (subunit II) | | | | | | |
| cytochrome caa3 oxidase | CtaD | BLi01707 | 1.02 | 1.01 | 3.13 | 3.68 |
| (subunit I) | | | | | | |
| cytochrome caa3 oxidase | CtaE | BLi01708 | 0.98 | 1.45 | 3.11 | 4.17 |
| (subunit III) | | | | | | |
| cytochrome caa3 oxidase | CtaF | BLi01709 | 0.85 | 0.96 | 4.00 | 3.42 |
| (subunit IV) | | | | | | |
| CtaG: function unknown | CtaG | BLi01710 | 0.97 | 1.18 | 3.02 | 4.46 |
| cytochrome bd ubiquinol | CydA | BLi04134 | 1.41 | 1.16 | 5.78 | 6.05 |
| oxidase (subunit I) | | | | | | |
| cytochrome bd ubiquinol | CydB | BLi04133 | 1.46 | 1.02 | 3.25 | 4.58 |
| oxidase (subunit II) | | | | | | |
| nitrate reductase (alpha | NarG | BLi02074 | 2.90 | 2.24 | 2.79 | 4.58 |
| subunit) | | | | | | |
| nitrate reductase (beta subunit) | NarH | BLi02073 | 2.51 | 1.20 | 3.09 | 6.01 |
| nitrate reductase (gamma | NarI | BLi02071 | 2.20 | 1.82 | 3.75 | 5.69 |
| subunit) | | | | | | |
| FMN-containing NADPH-linked | NfrA | BLi04022 | 3.87 | 3.40 | 2.69 | 3.58 |
| nitro/flavin reductase | | | | | | |
| menaquinolcytochrome c | QcrA | BLi02391 | 1.98 | 1.58 | 5.04 | 5.72 |
| oxidoreductase (iron-sulfur | | | | | | |
| subunit) | | | | | | |
| menaquinolcytochrome c | QcrB | BLi02390 | 2.11 | 1.95 | 5.60 | 7.40 |
| oxidoreductase (cytochrome b | | | | | | |
| subunit) | | | | | | |
| menaquinolcytochrome c | QcrC | BLi02389 | 1.35 | 1.45 | 4.42 | 7.06 |
| oxidoreductase (cytochrome | | | | | | |
| b/c subunit) | | | | | | |
| cytochrome aa3 quinol oxidase | QoxB | BLi04039 | 0.28 | 0.33 | 1.56 | 3.66 |
| (subunit I) | | | | | | |
| cytochrome aa3 quinol oxidase | QoxC | BLi04038 | 0.23 | 0.47 | 1.49 | 3.11 |
| (subunit III) | | | | | | |
| essential protein similar to | ResA | BLi02461 | 1.52 | 1.51 | 2.98 | 3.46 |
| cytochrome c biogenesis | | | | | | |
| protein | | | | | | |
| essential protein required for | ResB | BLi02460 | 1.14 | 1.12 | 1.94 | 3.18 |
| cytochrome c synthesis | | | | | | |
| two-component response | ResD | BLi02458 | 1.40 | 1.34 | 2.53 | 3.20 |
| regulator involved in aerobic | | | | | | |
| and anaerobic respiration | | | | | | |
| similar to NAD(P)H-flavin | YfkO | BLi00813 | 3.19 | 1.84 | 2.23 | 3.33 |
| oxidoreductase | | | | | | |
| similar to NADH-dependent | YqjM | BLi02551 | 3.49 | 3.04 | 1.90 | 2.66 |
| flavin oxidoreductase | | | | | | |
| 6-phosphofructokinase | PfkA | BLi03068 | 3.32 | 2.67 | 3.10 | 3.97 |
| glucose-6-phosphate | Pgi | BLi03314 | 3.49 | 1.95 | 3.04 | 4.08 |
| isomerase | | | | | | |
| glutamyl endopeptidase | Mpr | BLi00340 | 3.15 | 1.97 | 3.82 | 5.40 |
| precursor (glutamate specific | | | | | | |
| endopeptidase) | | | | | | |
| N-acetylglutamate gamma- | ArgC | BLi01206 | 4.38 | 1.20 | 0.18 | 0.27 |
| semialdehyde dehydrogenase | | | | | | |
| N-acetylornithine | ArgD | BLi01209 | 3.10 | 1.16 | 0.13 | 0.39 |
| aminotransferase | | | | | | |
| argininosuccinate lyase | ArgH | BLi03083 | 2.94 | 0.62 | 0.12 | 0.22 |
| ornithine acetyltransferase/ | ArgJ | BLi01207 | 4.01 | 0.80 | 0.20 | 0.30 |
| amino-acid acetyltransferase | | | | | | |
| bacillopeptidase F | Bpr | BLi01748 | 3.59 | 3.59 | 6.18 | 6.92 |
| major intracellular serine | IspA | BLi01423 | 1.51 | 4.42 | 4.26 | 4.15 |
| protease | | | | | | |
| homoserine O- | MetA | BLi02329 | 3.42 | 1.69 | 2.12 | 3.32 |
| succinyltransferase | | | | | | |
| assimilatory nitrate reductase | NasC | BLi00483 | 1.24 | 1.68 | 3.15 | 3.18 |
| (catalytic subunit) | | | | | | |
| assimilatory nitrite reductase | NasD | BLi00484 | 1.56 | 6.21 | 6.04 | 7.34 |
| (subunit) | | | | | | |
| assimilatory nitrite reductase | NasE | BLi00485 | 1.90 | 6.21 | 9.99 | 11.51 |
| (subunit) | | | | | | |
| putative hydroxybenzoate | | BLi03989 | 3.11 | 6.66 | 5.49 | 5.38 |
| hydroxylase | | | | | | |
| similar to proline oxidase | YcgM | BLi00373 | 1.76 | 1.37 | 2.38 | 4.43 |
| similar to 1-pyrroline-5- | YcgN | BLi00374 | 1.82 | 2.24 | 1.59 | 3.49 |
| carboxylate dehydrogenase | | | | | | |
| similar to oligoendopeptidase | YjbG | BLi01247 | 3.11 | 3.44 | 1.67 | 1.27 |
| similar to dehydrogenase | YrbE | BLi00809 | 11.66 | 8.81 | 10.05 | 19.61 |
| similar to aspartate | YwfG | BLi04237 | 3.06 | 3.24 | 5.28 | 6.33 |
| aminotransferase | | | | | | |
| similar to gamma- | YwrD | BLi03850 | 4.13 | 3.47 | 5.31 | 4.38 |
| glutamyltransferase | | | | | | |
| similar to 2′,3′-cyclic-nucleotide | YfkN | BLi00814 | 19.67 | 25.85 | 21.76 | 22.34 |
| 2′-phosphodiesterase | | | | | | |
| similar to 5′-nucleotidase | YhcR | BLi00982 | 5.60 | 8.21 | 5.61 | 10.02 |
| similar to ribonuclease | YurI | BLi03441 | 3.35 | 7.25 | 13.30 | 14.44 |
| | | | 4.84 | 9.45 | 12.64 | 12.78 |
| homolog to DhaS aldehyde | homolog | BLi03994 | 11.67 | 19.72 | 30.68 | 20.75 |
| dehydrogenase | to DhaS | | | | | |
| aldehyde dehydrogenase | DhaS | BLi02249 | 3.99 | 6.59 | 9.05 | 19.38 |
| gamma- | Ggt | BLi01364 | 1.48 | 1.57 | 3.48 | 3.99 |
| glutamyltranspeptidase | | | | | | |
| glutamyl-tRNA reductase | HemA | BLi02947 | 0.84 | 1.45 | 3.23 | 3.84 |
| uroporphyrin-III C- | NasF | BLi00486 | 3.52 | 3.20 | 9.27 | 8.03 |
| methyltransferase | | | | | | |
| probable thiamine- | ThiL | BLi00611 | 3.68 | 3.19 | 2.19 | 1.32 |
| monophosphate kinase | | | | | | |
| alkaline phosphatase III | PhoB | BLi02565 | 61.68 | 77.17 | 88.31 | 101.89 |
| phosphodiesterase/alkaline | PhoD | BLi00281 | 21.20 | 23.68 | 40.10 | 51.20 |
| phosphatase | | | | | | |
| phosphate starvation-induced | PhoH | BLi02725 | 3.35 | 2.04 | 1.69 | 3.67 |
| protein | | | | | | |
| two-component response | PhoP | BLi03059 | 2.31 | 3.02 | 1.89 | 1.4 |
| regulator involved in | | | | | | |
| phosphate regulation | | | | | | |
| two-component sensor | PhoR | BLi03058 | 2.61 | 3.25 | 1.99 | 1.78 |
| histidine kinase involved in | | | | | | |
| phosphate regulation | | | | | | |
| D-alanyl-aminopeptidase | DppA | BLi01392 | 1.22 | 1.24 | 3.36 | 3.40 |
| similar to peptide methionine | YppQ | BLi02302 | 1.86 | 1.90 | 3.02 | 3.33 |
| sulfoxide reductase | | | | | | |
| protein secretion (post- | PrsA | BLi01072 | 1.98 | 2.52 | 2.45 | 3.30 |
| translocation molecular | | | | | | |
| chaperone) | | | | | | |
| signal peptidase I | SipV | BLi01122 | 0.79 | 1.16 | 0.84 | 3.14 |
| component of the twin-arginine | TatCD | BLi00283 | 3.38 | 3.185 | 4.76 | 10.83 |
| translocation pathway | | | | | | |
| alpha-acetolactate | AlsD | BLi03847 | 18.09 | 13.07 | 14.39 | 23.12 |
| decarboxylase | | | | | | |
| alpha-acetolactate synthase | AlsS | BLi03848 | 40.54 | 19.19 | 30.12 | 42.42 |
| beta-galactosidase | LacA | BLi04277 | 3.43 | 3.59 | 4.32 | 3.59 |
| glucose 1-dehydrogenase | Gdh | BLi02566 | 13.10 | 7.32 | 12.10 | 24.57 |
| phytase | Phy | BLi00448 | 22.05 | 32.38 | 40.47 | 20.41 |
| xylose isomerase | XylA | BLi03559 | 3.09 | 1.35 | 2.15 | 2.99 |
| similar to glucose 1- | YhdF | BLi01012 | 3.69 | 4.37 | 2.31 | 3.12 |
| dehydrogenase | | | | | | |
| similar to plant metabolite | YtbE | BLi02838 | 4.49 | 3.42 | 1.51 | 3.92 |
| dehydrogenase | | | | | | |
| RNA polymerase sigma-F | SigF | BLi02495 | 3.59 | 3.54 | 4.71 | 5.07 |
| factor (stage II sporulation | | | | | | |
| protein AC) (sporulation sigma | | | | | | |
| factor) | | | | | | |
| anti-sigma F factor antagonist | SpoIIAA | BLi02497 | 4.07 | 5.63 | 8.65 | 12.85 |
| (stage II sporulation protein | | | | | | |
| AA) | | | | | | |
| anti-sigma F factor (stage II | SpoIIAB | BLi02496 | 6.06 | 10.94 | 12.36 | 20.57 |
| sporulation protein AB) | | | | | | |
| spore coat protein (outer) | CotE | BLi01927 | 6.44 | 3.92 | 4.00 | 13.71 |
| putative spore coat | | BLi00802 | 9.46 | 3.82 | 6.26 | 7.88 |
| polysaccharide synthesis | | | | | | |
| RNA polymerase sporulation | SigE | BLi01750 | 1.15 | 8.49 | 6.23 | 7.17 |
| mother cell-specific (early) | | | | | | |
| sigma factor | | | | | | |
| RNA polymerase sporulation | SigG | BLi01751 | 1.33 | 7.78 | 4.78 | 5.79 |
| forespore-specific (late) sigma | | | | | | |
| factor | | | | | | |
| two-component response | Spo0F | BLi03961 | 2.34 | 3.98 | 2.71 | 2.30 |
| regulator involved in the | | | | | | |
| initiation of sporulation | | | | | | |
| protease (processing of pro- | SpoIIGA | BLi01749 | 1.78 | 3.58 | 11.41 | 11.19 |
| sigma-E to active sigma-E) | | | | | | |
| mutants block sporulation after | SpoIIIAF | BLi02609 | 0.65 | 22.74 | 4.50 | 3.18 |
| engulfment | | | | | | |
| mutants block sporulation after | SpoIIIAG | BLi02608 | 0.75 | 17.94 | 7.23 | 6.55 |
| engulfment | | | | | | |
| mutants block sporulation after | SpoIIIAH | BLi02607 | 0.66 | 16.68 | 7.13 | 4.26 |
| engulfment | | | | | | |
| DNA translocase required for | SpoIIIE | BLi01906 | 5.42 | 3.65 | 4.67 | 2.31 |
| chromosome partitioning | | | | | | |
| through the septum into the | | | | | | |
| forespore | | | | | | |
| required for completion of | SpoIIQ | BLi03892 | 1.22 | 2.11 | 2.49 | 17.12 |
| engulfment | | | | | | |
| probable peptidyl-tRNA | spoVC | BLi00066 | 3.60 | 3.15 | 2.00 | 1.95 |
| hydrolase | | | | | | |
| required for spore cortex | spoVG | BLi00062 | 3.24 | 3.45 | 1.74 | 1.86 |
| synthesis | | | | | | |
| required for assembly of the | SpoVID | BLi02941 | 1.82 | 3.40 | 2.63 | 14.79 |
| spore coat | | | | | | |
| Stage III sporulation protein AA | SpoIIIAA | BLi02614 | 0.96 | 5.59 | 3.12 | 3 |
| Stage III sporulation protein AE | SpoIIIAE | BLi02610 | 1.21 | 5.12 | 2.89 | 3.21 |
| Stage V sporulation protein AB | SpoVAB | BLi02493 | 1.76 | 4.60 | 3.42 | 3.15 |
| fumarate hydratase | CitG | BLi03486 | 3.14 | 3.51 | 3.62 | 1.37 |
| citrate synthase II (major) | CitZ | BLi03062 | 3.94 | 2.30 | 2.00 | 2.37 |
| isocitrate dehydrogenase | Icd | BLi03061 | 3.54 | 3.33 | 1.72 | 1.32 |
| citrate synthase III | MmgD | BLi04094 | 2.12 | 1.71 | 3.39 | 3.93 |
| transcriptional regulator of the | AlsR | BLi03849 | 3.75 | 2.94 | 4.10 | 3.29 |
| alpha-acetolactate operon | | | | | | |
| transcriptional activator of the | BmrR | BLi02784 | 3.12 | 4.04 | 2.37 | 2.52 |
| bmrUR operon | | | | | | |
| transcriptional activator of | Mta | BLi00304 | 3.65 | 2.96 | 2.78 | 2.19 |
| multidrug-efflux transporter | | | | | | |
| genes | | | | | | |
| putative transcriptional | | BLi03995 | 3.20 | 6.10 | 6.68 | 7.72 |
| regulator | | | | | | |
| transcriptional antiterminator | SacT | BLi04018 | 1.41 | 5.40 | 3.83 | 5.05 |
| involved in positive regulation | | | | | | |
| of sacA and sacP | | | | | | |
| two-component response | Spo0A | BLi02593 | 2.09 | 3.71 | 2.34 | 2.52 |
| regulator central for the | | | | | | |
| initiation of sporulation | | | | | | |
| similar to transcriptional | YwrC | BLi03851 | 3.38 | 2.59 | 3.60 | 3.52 |
| regulator (Lrp/AsnC family) | | | | | | |
| ABC transporter required for | CydC | BLi04132 | 2.38 | 2.27 | 3.48 | 5.76 |
| expression of cytochrome bd | | | | | | |
| (ATP-binding protein) | | | | | | |
| ABC transporter required for | CydD | BLi04131 | 2.38 | 1.50 | 3.22 | 3.20 |
| expression of cytochrome bd | | | | | | |
| (ATP-binding protein) | | | | | | |
| D-alanyl-aminopeptidase | DppA | BLi01392 | 1.61 | 3.47 | 3.33 | 1.89 |
| dipeptide ABC transporter | DppB | BLi01393 | 1.37 | 3.41 | 4.85 | 1.98 |
| (permease) (sporulation) | | | | | | |
| dipeptide ABC transporter | DppC | BLi01394 | 1.63 | 4.27 | 3.04 | 3.26 |
| (permease) (sporulation) | | | | | | |
| dipeptide ABC transporter | DppE | BLi01396 | 1.50 | 4.32 | 5.46 | 5.04 |
| (dipeptide-binding protein) | | | | | | |
| (sporulation) | | | | | | |
| phosphate ABC transporter | PstA | BLi02674 | 2.40 | 11.79 | 4.29 | 3.73 |
| (permease) | | | | | | |
| PstBA: phosphate ABC | PstBA | BLi02673 | 3.19 | 28.70 | 6.04 | 5.32 |
| transporte | | | | | | |
| phosphate ABC transporter | PstBB | BLi02672 | 2.13 | 16.12 | 4.39 | 4.13 |
| (ATP-binding protein) | | | | | | |
| phosphate ABC transporter | PstC | BLi02675 | 2.91 | 26.52 | 4.78 | 5.07 |
| (permease); | | | | | | |
| phosphate ABC transporter | PstS | BLi02676 | 16.78 | 52.35 | 21.78 | 16.68 |
| (binding protein) | | | | | | |
| putative benzoate transport | | BLi03990 | 6.39 | 12.66 | 13.70 | 15.60 |
| protein | | | | | | |
| putative transporter | | BLi04241 | 1.93 | 3.46 | 4.28 | 4.66 |
| ribose ABC transporter (ribose- | RbsB | BLi03845 | 3.61 | 3.22 | 4.93 | 3.77 |
| binding protein) | | | | | | |
| similar to amino acid | YhdG | BLi00475 | 7.07 | 5.49 | 5.44 | 7.89 |
| transporter | | | | | | |
| similar to sodium-dependent | YhdH | BLi01013 | 1.49 | 2.21 | 1.80 | 3.34 |
| transporter | | | | | | |
| similar to phosphotransferase | YpqE | BLi02359 | 3.98 | 2.98 | 3.70 | 3.54 |
| system enzyme II | | | | | | |
| similar to potassium uptake | YuaA | BLi03264 | 3.01 | 4.59 | 3.06 | 2.14 |
| protein | | | | | | |
| similar to L-lactate permease | YvfH | BLi03677 | 4.68 | 3.64 | 3.40 | 3.42 |
| similar to multidrug transporter | YvmA | BLi03565 | 18.25 | | 12.23 | 12.80 |
| similar to iron transport system | YvrA | BLi03504 | 3.11 | 2.89 | 2.43 | 2.18 |
| similar to chromate transport | YwrB | BLi03852 | 3.21 | 1.96 | 3.95 | 5.06 |
| protein | | | | | | |
| RNA polymerase major sigma | SigA | BLi02712 | 1.07 | 1.63 | 4.50 | 4.17 |
| factor | | | | | | |
| RNA polymerase sigma factor | SigI | BLi01499 | 1.77 | 2.73 | 3.00 | 3.59 |
| hypothetical | | BLi02543 | 1.73 | 3.89 | 5.01 | 3.58 |
| conserved hypothetical | | BLi00216 | 3.25 | 4.13 | 1.72 | 1.92 |
| putative serine protease | | BLi00301 | 3.21 | 2.28 | 2.77 | 3.20 |
| putative acetoin reductase | | BLi02066 | 1.96 | 2.04 | 8.49 | 10.92 |
| hypothetical | | BLi00630 | 1.18 | 1.45 | 3.62 | 3.11 |
| putative acetyltransferase | | BLi02012 | 1.06 | 1.60 | 4.43 | 3.56 |
| putative aromatic compounds | | BLi03991 | 5.93 | 15.19 | 10.19 | 13.00 |
| specific dioxygenase | | | | | | |
| putative | | BLi03993 | 9.40 | 19.87 | 11.90 | 14.36 |
| decarboxylase/dehydratase | | | | | | |
| putative transcriptional | | BLi03995 | 3.20 | 3.40 | 6.52 | 7.72 |
| regulator | | | | | | |
| conserved hypothetical protein | | BLi03996 | 3.03 | 3.70 | 5.17 | 4.30 |
| putative phage protein | | BLi01466 | 1.69 | 5.35 | 3.36 | 4.26 |
| putative portal protein | | BLi01465 | 3.48 | 4.06 | 4.78 | 4.30 |
| penicillinase repressor | | BLi00279 | 3.25 | 3.00 | 3.38 | 3.94 |
| regulatory protein blaR1 | | BLi00278 | 3.37 | 3.25 | 3.24 | 3.70 |
| putative enzyme IICB | | BLi00268 | 2.14 | 3.22 | 3.55 | 5.27 |
| component | | | | | | |
| putative PTS system, | | BLi02561 | 2.44 | 3.23 | 2.93 | 3.67 |
| cellobiose-specific enzyme II, | | | | | | |
| C component | | | | | | |
| hypothetical protein | | BLi00236 | 3.72 | 1.42 | 4.77 | 3.38 |
| lichenysin synthetase A | | BLi00401 | 2.65 | 4.60 | 4.86 | 4.58 |
| conserved hypothetical protein | | BLi03193 | 14.04 | 10.10 | 10.27 | 11.34 |
| putative glucosyl transferase | | BLi03194 | 4.10 | 4.23 | 3.77 | 3.40 |
| (ADI) (Arginine dihydrolase) | | BLi04163 | 2.29 | 2.18 | 4.28 | 4.15 |
| (AD) | | | | | | |
| putative sugar permease | | BLi04165 | 1.43 | 1.34 | 3.51 | 3.02 |
| carbamate kinase | | BLi04166 | 1.07 | 1.52 | 3.37 | 3.56 |
| hypothetical | | BLi04184 | 4.89 | 2.13 | 6.55 | 6.42 |
| hypothetical | | BLi04185 | 3.48 | 3.04 | 2.70 | 3.69 |
| putative hydrolase | | BLi02233 | 1.1 | 3.30 | 3.42 | 4.14 |
| hypothetical | | BLi00811 | 3.21 | 2.03 | 2.75 | 5.22 |
| putative lipopolysaccharide | | BLi00807 | 3.42 | 3.39 | 3.15 | 3.62 |
| biosynthesis | | | | | | |
| putative spore coat | | BLi00802 | 9.46 | 6.73 | 6.26 | 7.88 |
| polysaccharide synthesis | | | | | | |
| putative ABC transporter ATP- | | BLi04117 | 3.36 | 3.12 | 1.36 | 1.52 |
| binding protein | | | | | | |
| conserved hypothetical protein | | BLi04120 | 3.66 | 3.08 | 1.36 | 1.67 |
| putative bacteriocin formation | | BLi04128 | 2.69 | 3.18 | 3.60 | 4.65 |
| protein | | | | | | |
| putative permease | | BLi02300 | 1.58 | 3.36 | 3.02 | 3.01 |
| putative oxidoreductase | | BLi01011 | 4.72 | 3.93 | 2.71 | 3.15 |
| conserved hypothetical protein | | BLi03108 | 3.40 | 3.71 | 3.57 | 2.87 |
| putative transcriptional | | BLi03548 | 3.58 | 2.15 | 3.30 | 1.92 |
| regulator | | | | | | |
| putative intracellular proteinase I | | BLi03556 | 3.50 | 3.34 | 3.08 | 4.31 |
| putative two-component hybrid | | BLi00981 | 3.65 | 3.34 | 3.01 | 3.28 |
| sensor and regulator | | | | | | |
| putative hydrolase | | BLi02785 | 1.06 | 1.48 | 3.56 | 4.45 |
| putative oxidoreductase | | BLi02819 | 4.05 | 3.34 | 4.08 | 8.87 |
| protein | | | | | | |
| putative phosphatase | | BLi02820 | 22.93 | 25.79 | 22.15 | 34.59 |
| putative lipase/esterase | | BLi02821 | 3.01 | 2.32 | 3.03 | 3.92 |
| hypothetical protein | | BLi02206 | 1.27 | 2.65 | 5.41 | 4.26 |
| putative ABC transporter ATP- | | BLi04307 | 2.61 | 2.03 | 3.42 | 5.94 |
| binding protein | | | | | | |
| conserved hypothetical protein | | BLi04240 | 2.32 | 1.64 | 3.81 | 5.11 |
| hypothetical protein | | BLi04238 | 2.95 | 1.84 | 4.36 | 5.59 |
| hypothetical protein | | BLi03570 | 4.00 | 1.62 | 4.45 | 6.10 |
| putative amidase | | BLi03671 | 3.36 | 1.28 | 5.23 | 4.23 |
| hypothetical protein | | BLi04308 | 3.58 | 2.42 | 6.94 | 16.93 |
| conserved hypothetical protein | | BLi02704 | 4.36 | 4.25 | 4.02 | 5.83 |
| hypothetical protein | | BLi03595 | 5.09 | 2.91 | 3.43 | 3.83 |
| hypothetical protein | | BLi00235 | 6.93 | 7.10 | 8.22 | 13.49 |
| similar to proteins | YdtG | BLi04370 | 3.18 | 2.77 | 3.38 | 4.49 |
| similar to proteins | YfkH | BLi00820 | 15.02 | 4.44 | 10.91 | 19.99 |
| similar to proteins | YfkM | BLi00815 | 4.18 | 3.84 | 3.77 | 1.82 |
| unknown | YfmQ | BLi00629 | 3.24 | 1.45 | 19.36 | 26.62 |
| similar to proteins from B. subtilis | YhbD | BLi00958 | 0.64 | 0.73 | 4.53 | 13.60 |
| similar to proteins from B. subtilis | YhbE | BLi00959 | 0.86 | 1.01 | 3.29 | 15.30 |
| similar to proteins from B. subtilis | YhbF | BLi00960 | 0.87 | 1.41 | 3.48 | 7.41 |
| similar to proteins | YjbC | BLi01237 | 3.11 | 3.23 | 2.86 | 3.27 |
| similar to proteins | YjoA | BLi02905 | 4.37 | 3.24 | 5.47 | 7.64 |
| similar to proteins | YlbA | BLi01711 | 3.26 | 1.98 | 3.41 | 3.76 |
| similar to proteins | YllB | BLi01730 | 3.06 | 3.17 | 2.92 | 3.57 |
| similar to proteins | YlxA | BLi01731 | 3.13 | 1.94 | 2.45 | 3.21 |
| similar to proteins from B. subtilis | YndM | BLi02243 | 3.20 | 3.14 | 2.52 | 3.26 |
| similar to proteins | YneR | BLi02056 | 1.79 | 3.54 | 2.76 | 3.24 |
| similar to proteins | YngK | BLi02129 | 1.83 | 1.06 | 3.44 | 3.07 |
| similar to proteins | YpiB | BLi02393 | 3.40 | 3.34 | 3.83 | 5.00 |
| similar to proteins | YpiF | BLi02392 | 3.19 | 3.14 | 3.41 | 3.52 |
| similar to proteins | YppE | BLi02392 | 2.45 | 2.06 | 3.47 | 6.18 |
| similar to proteins | YqxD | BLi02714 | 3.11 | 2.09 | 3.32 | 3.35 |
| similar to proteins | YrrK | BLi02867 | 3.56 | 3.12 | 3.29 | 3.66 |
| similar to proteins | YtkA | BLi03208 | 1.9 | 2.36 | 4.19 | 4.32 |
| similar to proteins from B. subtilis | YuaB | BLi03999 | 2.64 | 2.97 | 3.42 | 5.14 |
| unknown | YuaE | BLi03267 | 3.26 | 3.12 | 2.66 | 3.13 |
| similar to proteins | YunA | BLi03423 | 1.49 | 1.24 | 3.54 | 3.64 |
| unknown | YusD | BLi03458 | 1.81 | 1.73 | 3.26 | 3.12 |
| YvaA: unknown; similar to | YvaA | BLi03572 | 3.15 | 1.87 | 3.31 | 3.81 |
| unkno | | | | | | |
| similar to proteins | YvmC | BLi03566 | 26.40 | 1.72 | 103.05 | 149.62 |
| similar to proteins | YvnA | BLi03569 | 50.99 | 1.14 | 118.91 | 112.22 |
| similar to proteins from B. subtilis | YvqH | BLi03496 | 4.67 | 3.25 | 3.88 | 3.47 |
| unknown | YwfB | BLi04239 | 3.84 | 1.54 | 3.79 | 4.91 |
| similar to proteins | Ywfl | BLi03998 | 2.44 | 2.26 | 3.06 | 3.18 |
| similar to proteins | YwfL | BLi03988 | 2.84 | 6.59 | 5.92 | 6.33 |
| similar to proteins | YwiC | BLi02079 | 1.46 | 1.14 | 4.24 | 6.38 |

### Example 3

Genes which are Markedly Induced Especially Under Phosphate Deficiency

Table 2 below lists all the Bacillus licheniformis DSM13 genes determined in Example 1, whose induction under the conditions of phosphate deficiency described in Example 1 has been at least a factor of 10 at any of the times of measurement and which may be classified as comparatively specific for phosphate deficiency on the basis of comparative experiments (data not shown). These are 47 genes in total.

The column headers are the same as in the preceding example. In addition, the first column indicates the sequence numbers of the particular DNA and amino acid sequences in the sequence listing of the present application. Specific features of the particular sequences, which appear as free text in the sequence listing have been added under the heading Gene name/gene function.

TABLE 2
The 47 Bacillus licheniformis DSM13 genes determined in
Example 1, whose induction caused especially by phosphate
deficiency at any of the times of measurement has been at least a
factor of 10 (explanations: see text).

| SEQ | | | | | | | |
|---|---|---|---|---|---|---|---|
| ID NO. | Gene name/gene function | ID | Bli-No. | Transition | 0.5 h | 1 h | 2 h |
| 1. 2 | class III heat-shock | HtpG | BLi04256 | 19.93 | 4.26 | 3.42 | 4.35 |
| | protein | | | | | | |
| 3, 4 | cytochrome P450-like | CypX | BLi03567 | 15.40 | 1.65 | 37.31 | 41.21 |
| | enzyme | | | | | | |
| 5, 6 | cytochrome caa3 | CtaC | BLi01706 | 1.05 | 1.29 | 7.62 | 10.87 |
| | oxidase (subunit II) | | | | | | |
| 7, 8 | assimilatory nitrite | NasE | BLi00485 | 1.90 | 6.21 | 9.99 | 11.51 |
| | reductase (subunit) | | | | | | |
| 9, 10 | similar to | YrbE | BLi00809 | 11.66 | 8.81 | 10.05 | 19.61 |
| | dehydrogenase; | | | | | | |
| | GTG start codon (“First | | | | | | |
| | codon translated as | | | | | | |
| | Met.”) | | | | | | |
| 11, 12 | similar to 2′,3′-cyclic- | YfkN | BLi00814 | 19.67 | 25.85 | 21.76 | 22.34 |
| | nucleotide 2′- | | | | | | |
| | phosphodiesterase | | | | | | |
| 13, 14 | similar to 5′- | YhcR | BLi00982 | 5.60 | 8.21 | 5.61 | 10.02 |
| | nucleotidase | | | | | | |
| 15, 16 | similar to ribonuclease | YurI | BLi03441 | 3.35 | 7.25 | 13.30 | 14.44 |
| 17, 18 | homolog to DhaS | homolog | BLi03994 | 11.67 | 19.72 | 30.68 | 20.75 |
| | aldehyde | to DhaS | | | | | |
| | dehydrogenase | | | | | | |
| 19, 20 | aldehyde | DhaS | BLi02249 | 3.99 | 6.59 | 9.05 | 19.38 |
| | dehydrogenase; | | | | | | |
| | Startcodon TTG (“First | | | | | | |
| | codon translated as | | | | | | |
| | Met.”) | | | | | | |
| 21, 22 | alkaline phosphatase III | PhoB | BLi02565 | 61.68 | 77.17 | 88.31 | 101.89 |
| 23, 24 | phosphodiesterase/alkaline | PhoD | BLi00281 | 21.20 | 23.68 | 40.10 | 51.20 |
| | phosphatase | | | | | | |
| 25, 26 | component of the twin- | TatCD | BLi00283 | 3.38 | 3.185 | 4.76 | 10.83 |
| | arginine translocation | | | | | | |
| | pathway; | | | | | | |
| | GTG start codon (“First | | | | | | |
| | codon translated as | | | | | | |
| | Met”.) | | | | | | |
| 27, 28 | alpha-acetolactate | AlsD | BLi03847 | 18.09 | 13.07 | 14.39 | 23.12 |
| | decarboxylase | | | | | | |
| 29, 30 | alpha-acetolactate | AlsS | BLi03848 | 40.54 | 19.19 | 30.12 | 42.42 |
| | synthase; | | | | | | |
| | Startcodon TTG (“First | | | | | | |
| | codon translated as | | | | | | |
| | Met.”) | | | | | | |
| 31, 32 | glucose 1- | Gdh | BLi02566 | 13.10 | 7.32 | 12.10 | 24.57 |
| | dehydrogenase | | | | | | |
| 33, 34 | phytase | Phy | BLi00448 | 22.05 | 32.38 | 40.47 | 20.41 |
| 35, 36 | anti-sigma F factor | SpoIIAA | BLi02497 | 4.07 | 5.63 | 8.65 | 12.85 |
| | antagonist (stage II | | | | | | |
| | sporulation protein AA) | | | | | | |
| 37, 38 | anti-sigma F factor | SpoIIAB | BLi02496 | 6.06 | 10.94 | 12.36 | 20.57 |
| | (stage II sporulation | | | | | | |
| | protein AB) | | | | | | |
| 39, 40 | spore coat protein | CotE | BLi01927 | 6.44 | 3.92 | 4.00 | 13.71 |
| | (outer) | | | | | | |
| 41, 42 | protease (processing of | SpoIIGA | BLi01749 | 1.78 | 3.58 | 11.41 | 11.19 |
| | pro-sigma-E to active | | | | | | |
| | sigma-E); | | | | | | |
| | GTG start codon (“First | | | | | | |
| | codon translated as | | | | | | |
| | Met.”) | | | | | | |
| 43, 44 | required for completion | SpoIIQ | BLi03892 | 1.22 | 2.11 | 2.49 | 17.12 |
| | of engulfment | | | | | | |
| 45, 46 | required for assembly of | SpoVID | BLi02941 | 1.82 | 3.40 | 2.63 | 14.79 |
| | the spore coat; | | | | | | |
| | Startcodon TTG (“First | | | | | | |
| | codon translated as | | | | | | |
| | Met.”) | | | | | | |
| 47, 48 | phosphate ABC | PstS | BLi02676 | 16.78 | 52.35 | 21.78 | 16.68 |
| | transporter (binding | | | | | | |
| | protein); | | | | | | |
| | GTG start codon (“First | | | | | | |
| | codon translated as | | | | | | |
| | Met.”) | | | | | | |
| 49, 50 | putative benzoate | | BLi03990 | 6.39 | 12.66 | 13.70 | 15.60 |
| | transport protein | | | | | | |
| 51, 52 | similar to multidrug | YvmA | BLi03565 | 18.25 | | 12.23 | 12.80 |
| | transporter | | | | | | |
| 53, 54 | putative acetoin | | BLi02066 | 1.96 | 2.04 | 8.49 | 10.92 |
| | reductase | | | | | | |
| 55, 56 | putative aromatic | | BLi03991 | 5.93 | 15.19 | 10.19 | 13.00 |
| | compounds specific | | | | | | |
| | dioxygenase | | | | | | |
| 57, 58 | putative | | BLi03993 | 9.40 | 19.87 | 11.90 | 14.36 |
| | decarboxylase/dehydratase | | | | | | |
| 59, 60 | conserved hypothetical | | BLi03193 | 14.04 | 10.10 | 10.27 | 11.34 |
| | protein | | | | | | |
| 61, 62 | putative phosphatase; | | BLi02820 | 22.93 | 25.79 | 22.15 | 34.59 |
| | GTG start codon (“First | | | | | | |
| | codon translated as | | | | | | |
| | Met.”) | | | | | | |
| 63, 64 | hypothetical protein | | BLi04308 | 3.58 | 2.42 | 6.94 | 16.93 |
| 65, 66 | hypothetical protein | | BLi00235 | 6.93 | 7.10 | 8.22 | 13.49 |
| 67, 68 | similar to proteins | YfkH | BLi00820 | 15.02 | 4.44 | 10.91 | 19.99 |
| 69, 70 | unknown; | YfmQ | BLi00629 | 3.24 | 1.45 | 19.36 | 26.62 |
| | GTG start codon (“First | | | | | | |
| | codon translated as | | | | | | |
| | Met.”) | | | | | | |
| 71, 72 | similar to proteins from | YhbD | BLi00958 | 0.64 | 0.73 | 4.53 | 13.60 |
| | B. subtilis | | | | | | |
| 73, 74 | similar to proteins from | YhbE | BLi00959 | 0.86 | 1.01 | 3.29 | 15.30 |
| | B. subtilis | | | | | | |
| 75, 76 | similar to proteins | YvmC | BLi03566 | 26.40 | 1.72 | 103.05 | 149.62 |
| 77, 78 | similar to proteins from | YvnA | BLi03569 | 50.99 | 1.14 | 118.91 | 112.22 |
| | B. subtilis | | | | | | |
| 79, 80 | mutants block | SpoIIIAF | BLi02609 | 0.65 | 22.74 | 4.50 | 3.18 |
| | sporulation after | | | | | | |
| | engulfment | | | | | | |
| 81, 82 | mutants block | SpoIIIAG | BLi02608 | 0.75 | 17.94 | 7.23 | 6.55 |
| | sporulation after | | | | | | |
| | engulfment | | | | | | |
| 83, 84 | mutants block | SpoIIIAH | BLi02607 | 0.66 | 16.68 | 7.13 | 4.26 |
| | sporulation after | | | | | | |
| | engulfment | | | | | | |
| 85, 86 | phosphate ABC | PstA | BLi02674 | 2.40 | 11.79 | 4.29 | 3.73 |
| | transporter (permease) | | | | | | |
| 87, 88 | PstBA: phosphate ABC | PstBA | BLi02673 | 3.19 | 28.70 | 6.04 | 5.32 |
| | transporte | | | | | | |
| 89, 90 | phosphate ABC | PstBB | BLi02672 | 2.13 | 16.12 | 4.39 | 4.13 |
| | transporter (ATP-binding | | | | | | |
| | protein) | | | | | | |
| 91, 92 | phosphate ABC | PstC | BLi02675 | 2.91 | 26.52 | 4.78 | 5.07 |
| | transporter (permease); | | | | | | |
| 93, 94 | putative ribonuclease; | | BLi03719 | 7.98 | 11.03 | 13.68 | 19.00 |
| | GTG start codon (“First | | | | | | |
| | codon translated as | | | | | | |
| | Met.”) | | | | | | |

Table 3 below lists the same 47 genes once more in the order of observed strength of induction. The corresponding terms in English are indicated in this order in the description section. The arrangement of the subject matter of the claims follows the reverse order.

TABLE 3
The 47 Bacillus licheniformis DSM13 genes determined in
Example 1, whose induction caused especially by phosphate
deficiency at any of the times of measurement has been at least a
factor of 10, in descending order of the maximum value (last
column) measured in each case.

| SEQ | | | | |
|---|---|---|---|---|
| ID | | | | |
| NO. | Gene name/gene function/Comments on the gene | Gene | Bli-No. | max. |
| 75, 76 | similar to proteins | yvmC | BLi03566 | 149.62 |
| | (similar to proteins of unknown function) | | | |
| 77, 78 | similar to proteins from B. subtilis | yvnA | BLi03569 | 118.91 |
| 21, 22 | alkaline phosphatase III | phoB | BLi02565 | 101.89 |
| 47, 48 | phosphate ABC transporter (binding protein); | pstS | BLi02676 | 52.35 |
| | GTG start codon; the first codon is | | | |
| | translated as methionine. | | | |
| 23, 24 | phosphodiesterase/alkaline phosphatase | phoD | BLi00281 | 51.20 |
| 29, 30 | alpha-acetolactate synthase; | alsS | BLi03848 | 42.42 |
| | TTG start codon; | | | |
| | the first codon is translated as methionine | | | |
| 3, 4 | cytochrome P450-like enzyme | cypX | BLi03567 | 41.21 |
| 33, 34 | phytase | phy | BLi00448 | 40.47 |
| 61, 62 | putative phosphatase; | | BLi02820 | 34.59 |
| | GTG start codon; the first | | | |
| | codon is translated as methionine. | | | |
| 17, 18 | homolog to DhaS aldehyde dehydrogenase | homolog | BLi03994 | 30.68 |
| | | to dhaS | | |
| 87, 88 | PstBA: phosphate ABC transporter | pstBA | BLi02673 | 28.70 |
| 69, 70 | GTG start codon; the first | yfmQ | BLi00629 | 26.62 |
| | codon is translated as methionine. | | | |
| | (unknown function) | | | |
| 91, 92 | phosphate ABC transporter (permease) | pstC | BLi02675 | 26.52 |
| 11, 12 | similar to 2′,3′-cyclic-nucleotide 2′- | yfkN | BLi00814 | 25.85 |
| | phosphodiesterase | | | |
| 31, 32 | glucose 1-dehydrogenase | gdh | BLi02566 | 24.57 |
| 27, 28 | alpha-acetolactate decarboxylase | alsD | BLi03847 | 23.12 |
| 79, 80 | mutants block sporulation after engulfment | spoIIIAF | BLi02609 | 22.74 |
| | (sporulation factor III AF) | | | |
| 37, 38 | anti-sigma F factor (stage II sporulation protein AB) | spoIIAB | BLi02496 | 20.57 |
| 67, 68 | similar to proteins | yfkH | BLi00820 | 19.99 |
| 1, 2 | class III heat-shock protein | htpG | BLi04256 | 19.93 |
| 57, 58 | putative decarboxylase/dehydratase | | BLi03993 | 19.87 |
| 9, 10 | similar to dehydrogenase; | yrbE | BLi00809 | 19.61 |
| | GTG start codon; the first | | | |
| | codon is translated as methionine. | | | |
| 19, 20 | aldehyde dehydrogenase; | dhaS | BLi02249 | 19.38 |
| | GTG start codon; the first | | | |
| | codon is translated as methionine. | | | |
| 93, 94 | putative ribonuclease; | | BLi03719 | 19.00 |
| | GTG start codon; the first | | | |
| | codon is translated as methionine. | | | |
| 51, 52 | similar to multidrug transporter | yvmA | BLi03565 | 18.25 |
| 81, 82 | mutants block sporulation after engulfment | spoIIIAG | BLi02608 | 17.94 |
| | (sporulation factor III AG) | | | |
| 43, 44 | required for completion of engulfment | spoIIQ | BLi03892 | 17.12 |
| | (sporulation factor II Q) | | | |
| 63, 64 | hypothetical protein | | BLi04308 | 16.93 |
| 83, 84 | mutants block sporulation after engulfment | spoIIIAH | BLi02607 | 16.68 |
| | (sporulation factor III AH) | | | |
| 89, 90 | phosphate ABC transporter (ATP-binding protein) | pstBB | BLi02672 | 16.12 |
| 49, 50 | putative benzoate transport protein | | BLi03990 | 15.60 |
| 73, 74 | similar to proteins from B. subtilis | yhbE | BLi00959 | 15.30 |
| 55, 56 | putative aromatic compounds specific dioxygenase | | BLi03991 | 15.19 |
| 45, 46 | required for assembly of the spore coat; | spoVID | BLi02941 | 14.79 |
| | TTG start codon; the first codon | | | |
| | is translated as methionine. | | | |
| | (sporulation factor VI D) | | | |
| 15, 16 | similar to ribonuclease | yurI | BLi03441 | 14.44 |
| 59, 60 | conserved hypothetical protein | | BLi03193 | 14.04 |
| 39, 40 | spore coat protein (outer) | cotE | BLi01927 | 13.71 |
| 71, 72 | similar to proteins from B. subtilis | yhbD | BLi00958 | 13.60 |
| 65, 66 | hypothetical protein | | BLi00235 | 13.49 |
| 35, 36 | anti-sigma F factor antagonist (stage II sporulation | spoIIAA | BLi02497 | 12.85 |
| | protein AA) | | | |
| 85, 86 | phosphate ABC transporter (permease) | pstA | BLi02674 | 11.79 |
| 7, 8 | assimilatory nitrite reductase (subunit) | nasE | BLi00485 | 11.51 |
| 41, 42 | protease (processing of pro-sigma-E to active | spoIIGA | BLi01749 | 11.41 |
| | sigma-E); | | | |
| | GTG start codon; the first codon | | | |
| | is translated as methionine. | | | |
| | (sporulation factor II GA) | | | |
| 53, 54 | putative acetoin reductase | | BLi02066 | 10.92 |
| 5, 6 | cytochrome caa3 oxidase (subunit II) | ctaC | BLi01706 | 10.87 |
| 25, 26 | component of the twin-arginine translocation | tatCD | BLi00283 | 10.83 |
| | pathway; | | | |
| | GTG start codon; the first codon is | | | |
| | translated as methionine. | | | |
| 13, 14 | similar to 5′-nucleotidase | yhcR | BLi00982 | 10.02 |

### Example 4

Real Time RT-PCR Quantification of Selected Genes

Real time RT-PCR enables the absolute numbers of molecules of specific transcripts in a sample to be determined. The LightCycler (Roche Diagnostics, Penzberg, Germany) was utilized in combination with the “SYBR Green I” kit (Roche Diagnostics). This method is based on detecting the binding of the fluorescent dye to double-stranded DNA. The specific mRNA molecules were quantified as described in Tobisch et al. (2003; Quantification of Bacterial mRNA by One-Step RT-PCR Using the LightCycler System; BIOCHEMICA, Volume 3, pages 5 to 8), with an external standard curve being established for each mRNA to be studied.

For this purpose, dilutions of known concentrations of in vitro transcripts of the mRNAs determined in Example 1 and listed in Example 2 were measured with the aid of the LightCycler, following by establishing the standard curve using the apparatus-specific software. For this purpose, primers which are specific for each mRNA to be studied and which enable in vitro transcripts to be synthesized and PCR amplification in the LightCycler had to be selected beforehand (with the aid of the “Array Designer” software; available from PREMIER Biosoft International, Palo Alto, USA).

After generating the external standard curve, measurement in the LightCycler was started. Two different dilutions of each sample to be analyzed were used for the measurements. The particular primers were then used in the LightCycler run for amplifying the specific transcript, and incorporation of the dye was then measured.

Using the LightCycler software and the script accessible on the website htt://molbiol.ru/ger/scripts/01 07.html Skripts (12.2.2004), it was possible to determine the exact number of molecules for selected transcripts. The values obtained in this manner and their relations to one another were then used for confirming the induction values indicated in Tables 1 and 2 for the selected transcripts.

1. A nucleic acid-binding chip doped with phosphate-metabolism specific probes for at least three genes selected from the group consisting of yhcR, tatCD, ctaC, gene for a putative acetoin reductase (SEQ ID No. 53 homolog), spoIIGA, nasE, pstA, spoIIAA, gene for a hypothetical protein (SEQ ID No. 65 homolog), yhbD, cotE, gene for a conserved hypothetical protein (SEQ ID No. 59 homolog), yurl, spoVID, gene for a putative aromatic-specific dioxygenase (SEQ ID No. 55 homolog), yhbE, gene for a putative benzoate transport protein (SEQ ID No. 49 homolog), pstBB, spoIIIAH, gene for a hypothetical protein (SEQ ID No. 63 homolog), spoIIQ, spoIIIAG, yvmA, gene for a putative ribonuclease (SEQ ID No. 93 homolog), dhaS, yrbE, gene for a putative decarboxylase/dehydratase (SEQ ID No. 57 homolog), htpG, yfkH, spoIIAB, spoIIIAF, alsD, gdh, yfkN, pstC, yfmQ, pstBA, dhaS homolog (DhaS aldehyde dehydrogenase homolog; SEQ ID No. 17 homolog), gene for a putative phosphatase (SEQ ID No. 61 homolog), phy, cypX, alsS, phoD, pstS, phoB, yvnA, and yvmC;
the total number of all different phosphate metabolism-specific probes not exceeding 100.

2. A nucleic acid-binding chip according to claim 1, doped with probes for at least three genes selected from the group consisting of: gene for a hypothetical protein (SEQ ID No. 65 homolog), yhbD, cotE, gene for a conserved hypothetical protein (SEQ ID No. 59 homolog), yurl, spoVID, gene for a putative aromatic-specific dioxygenase (SEQ ID No. 55 homolog), yhbE, gene for a putative benzoate transport protein (SEQ ID No. 49 homolog), pstBB, spoIIIAH, gene for a hypothetical protein (SEQ ID No. 63 homolog), spoIIQ, spoIIIAG, yvmA, gene for a putative ribonuclease (SEQ ID No. 93 homolog), dhaS, yrbE, gene for a putative decarboxylase/dehydratase (SEQ ID No. 57 homolog), htpG, yfkH, spoIIAB, spoIIIAF, alsD, gdh, yfkN, pstC, yfmQ, pstBA, dhaS homolog (DhaS aldehyde dehydrogenase homolog; SEQ ID No. 17 homolog), gene for a putative phosphatase (SEQ ID No. 61 homolog), phy, cypX, alsS, phoD, pstS, phoB, yvnA, and yvmC.

3. A nucleic acid-binding chip according to claim 2, doped with probes for at least three genes selected from the group consisting of: yfkN, pstC, yfmQ, pstBA, dhaS homolog (DhaS aldehyde dehydrogenase homolog; SEQ ID No. 17 homolog), gene for a putative phosphatase (SEQ ID No. 61 homolog), phy, cypX, alsS, phoD, pstS, phoB, yvnA, and yvmC.

4. A nucleic acid-binding chip according to claim 3, doped with probes for at least three genes selected from the group consisting: phy, cypX, alsS, phoD, pstS, phoB, yvnA, and yvmC.

5. A nucleic acid-binding chip according to claim 4, doped with probes for at least one gene selected from the group consisting of: phoB, yvnA, and yvmC.

6. A nucleic acid-binding chip according to claim 1, wherein the total number of all different probes does not exceed 50.

7. A nucleic acid-binding chip according to claim 1, wherein the specified probes are those which react to the most homologous, in vivo-transcribable genes from an organism chosen for a predetermined bioprocess.

8. A nucleic acid-binding chip according to claim 1, wherein the organism selected for the bioprocess is a selected from the group consisting of unicellular eukaryotes, Gram-positive and Gram-negative bacteria.

9. A nucleic acid-binding chip according to claim 8, wherein the unicellular eukaryotes are yeast selected from the group consisting of Saccharomyces and Schizosaccharomyces.

10. A nucleic acid-binding chip according to claim 8, wherein the Gram-positive bacteria are selected from the group consisting of the species Staphylococcus carnosus, Corynebacterium glutamicum, Bacillus subtilis, B. licheniformis, B. amyloliquefaciens, B. agaradherens, B. stearothermophilus, B. globigii and B. lentus.

11. A nucleic acid-binding chip according to claim 8, wherein the Gram-negative bacteria are selected from the group consisting of derivatives of the strains Escherichia coli BL21 (DE3), E. coli RV308, E. coli DH5α, E. coli JM109, E. coli XL-1 and Klebsiella planticola (Rf).

12. A nucleic acid-binding chip according to claim 1, wherein at least one of the specified probes is derived from the sequences listed in the sequence listing under numbers SEQ ID No. 1, 3, 5, 7, 9, 11, 13, 15, 17,19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91 and 93.

13. A nucleic acid-binding chip according to claim 1, additionally doped with at least one probe for at least one additional gene, the at least one additional gene being metabolically associated with the gene(s) additionally expressed depending on a predetermined bioprocess.

14. A nucleic acid-binding chip according to claim 13, wherein the gene additionally expressed depending on the predetermined bioprocess is selected from the group consisting of amylases, cellulases, lipases, oxidoreductases, hemicellulases, proteases, products of genes on a synthetic pathway of a low molecular-weight chemical compound, and products of genes which at least partially regulates a synthetic pathway of a low molecular weight compound.

15. A nucleic acid-binding chip according to claim 1, wherein at least one of the specified probes is provided in single-stranded form in the form of the codogenic strand.

16. A nucleic acid-binding chip according to claim 1 wherein at least one of the specified probes is provided in the form of a DNA or a nucleic acid analog.

17. A nucleic acid-binding chip according to claim 1, wherein at least one of the specified probes comprises a gene region which is transcribed into mRNA by the organism to be studied.

18. A nucleic acid-binding chip according to claim 17, wherein the transcribed gene region is close to the 5′ end of said mRNA.

19. A nucleic acid-binding chip according to claim 1, wherein at least one probe reacts with fragments of nucleic acid corresponding to the at least one probe.

20. A nucleic acid-binding chip according to claim 19, wherein the fragments of nucleic acid corresponding to the at least one probe is mRNA have a low degree of secondary folding, based on total corresponding mRNA.

21. A nucleic acid-binding chip according to claim 1, wherein at least one of the specified probes has a length of less than 200 nucleotides.

22. A nucleic acid-binding chip according to claim 1, further comprising means for triggering an electric signal when mRNA binds to a corresponding at least one probe.

23. A method for determining the physiological state of an organism undergoing a biological process, the method comprising:
(a) providing at least one nucleic acid-binding chip comprising at least three probes for nucleic acid or nucleic acid analog, the probes being selected from the group consisting of probes for the genes yhcR, tatCD, ctaC, gene for a putative acetoin reductase (SEQ ID No. 53 homolog), spoIIGA, nasE, pstA, spoIIAA, gene for a hypothetical protein (SEQ ID No. 65 homolog), yhbD, cotE, gene for a conserved hypothetical protein (SEQ ID No. 59 homolog), yurl, spoVID, gene for a putative aromatic-specific dioxygenase (SEQ ID No. 55 homolog), yhbE, gene for a putative benzoate transport protein (SEQ ID No. 49 homolog), pstBB, spoIIIAH, gene for a hypothetical protein (SEQ ID No. 63 homolog), spoIIQ, spoIIIAG, yvmA, gene for a putative ribonuclease (SEQ ID No. 93 homolog), dhaS, yrbE, gene for a putative decarboxylase/dehydratase (SEQ ID No. 57 homolog), htpG, yfkH, spoIIAB, spoIIIAF, alsD, gdh, yfkN, pstC, yfmQ, pstBA, dhaS homolog (DhaS aldehyde dehydrogenase homolog; SEQ ID No. 17 homolog), gene for a putative phosphatase (SEQ ID No. 61 homolog), phy, cypX, alsS, phoD, pstS, phoB, yvnA, and yvmC; and
(b) applying a medium including nucleic acid from the organism to the at least one chip.

24. A method according to claim 23 wherein the at least three probes are selected from probes for the group of genes consisting: gene for a hypothetical protein (SEQ ID No. 65 homolog), yhbD, cotE, gene for a conserved hypothetical protein (SEQ ID No. 59 homolog), yurl, spoVID, gene for a putative aromatic-specific dioxygenase (SEQ ID No. 55 homolog), yhbE, gene for a putative benzoate transport protein (SEQ ID No. 49 homolog), pstBB, spoIIIAH, gene for a hypothetical protein (SEQ ID No. 63 homolog), spoIIQ, spoIIIAG, yvmA, gene for a putative ribonuclease (SEQ ID No. 93 homolog), dhaS, yrbE, gene for a putative decarboxylase/dehydratase (SEQ ID No. 57 homolog), htpG, yfkH, spoIIAB, spoIIIAF, alsD, gdh, yfkN, pstC, yfmQ, pstBA, dhaS homolog (DhaS aldehyde dehydrogenase homolog; SEQ ID No. 17 homolog), gene for a putative phosphatase (SEQ ID No. 61 homolog), phy, cypX, alsS, phoD, pstS, phoB, yvnA, yvmC, preferably for at least three of the following 14 genes: yfkN, pstC, yfmQ, pstBA, dhaS homolog (DhaS aldehyde dehydrogenase homolog; SEQ ID No. 17 homolog), gene for a putative phosphatase (SEQ ID No. 61 homolog), phy, cypX, alsS, phoD, pstS, phoB, yvnA, and yvmC.

25. A method according to claim 23 wherein the physiological state is the status of phosphate metabolism of the organism.

26. A method according to claim 25, wherein the change in the phosphate metabolism of the organism undergoing the biological process relates to a phosphate deficiency condition.

27. A method according to claim 23, wherein the at least one probe is derived from the a sequence listed in the sequence listing under the numbers SEQ ID No. 1, 3, 5, 7, 9,11,13,15,17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91 and 93.

28. A method according to claim 23, wherein the organism selected for the bioprocess is selected from the group consisting of unicellular eukaryotes, Gram-positive and Gram-negative bacteria.

29. A method according to claim 28, wherein the unicellular eukaryotes are yeast select from the group consisting of Saccharomyces and Schizosaccharomyces.

30. A method according to claim 28, wherein the Gram-positive bacteria is selected from the group consisting of the species Staphylococcus carnosus, Corynebacterium glutamicum, Bacillus subtilis, B. licheniformis, B. amyloliquefaciens, B. agaradherens, B. stearothermophilus, B. globigii and B. lentus.

31. A method according to claim 28, wherein the Gram-negative bacteria are selected from the derivatives of the strains Escherichia coli BL21 (DE3), E. coli RV308, E. coli DH5α, E. coli JM109, E. coli XL-1 and Klebsiella planticola (Rf).

32. A method according to claim 23, wherein the physiological state is determined at various points in time of the same biological process using the same nucleic acid-binding chip.

33. A method according to claim 23, wherein the physiological state is determined at various points in time of the same biological process using a plurality of nucleic acid-binding chips, each of the plurality of nucleic acid chips being constructed in the same way.

34. A method according to claim 23, wherein the biological process is a fermentation that produces a substance selected from the group consisting of proteins and low molecular-weight chemical compounds.

35. A method according to claim 34, wherein the low molecular-weight chemical compound is selected from the group consisting of natural substances, food supplements and pharmaceutically relevent compounds.

36. A method according to claim 34, wherein the protein is an enzyme selected from the group consisting of a α-amylases, proteases, cellulases, lipases, oxidoreductases, peroxidases, laccases, oxidases and hemicellulases.


SEQUENCE LISTING (94 sequences)

SEQ ID NO: 1 - DNA, 1881 bp, Bacillus licheniformis DSM 13
  gene (1)..(1881): htpG; class III heat-shock protein; BLi04256
  CDS (1)..(1881)
atggctaagagagaattcaaagcagaatccaagcgattgctggacatcatgatcaactcgatttattcccaaaaggaagtctttttaagggagctgatatccaatgcgagcgatgccatcgataaaatctactacaaagcgctgacagatgattcactgacatttaacaaagacgattactacataaaaatctccgcagataaagaaaacagaacgctgaccattgcggacaccggaatcggcatgacgaaagaagagcttgaagagcatcttggaacgattgccaagagcggctcgcttgcgtttaaacaagaaaacgaattaaaagacgggcacgatattatcggccagttcggcgtcggtttctacgccgcttttatggttgcggacaccgtcaccgtcattacaaaagcccatggcagcgatgaagcgcatcaatgggaatcagccggagccgacggctatacaatcgaaccggctgcgaaagaatcagccggaaccgatgtcattctgaagctgaaagagaatacagacgatgaaaactatgatgaatacctggatgttcatcgcttaaaagcgatcatcaaaacatactctgatttcatccgctacccgatcaagatggatgtcgcggtcaacaagccgaaagaaggcgccgaaaatgaatttgaagaagtccaggaagaacagacggtcaacagcatggtgccgatttggagaaaaaacaaaagcgaactgaaagatgaagattatgaggcgttttataaggaaaagcattacggttttgacaagcctctcgcgcacatccatacaagcgtagacggcgcggtgagataccatgcgattttattcattcctgaaaatattccgttcaactactacacgaaggaattcgagaagggtctggaactgtattcgaacggtgtgctgatcatggaaaagtgtccggatctgctgcctgaccatttcagctttgtcaaaggaatggttgattcagaagatttatcgctcaacatttcgagggagatgctccagcatgacaggcagctgaagctgattgccaaaaacatcagcaaaaaaatcaagaatgagctgaagagcttgctgaaaaatgacagagaaaaatacgagtctttttatcaatcgtttggcagacagcttaaattcggggtatacaatgattttggagcccataaagatctgttgaaagacctgctgcttttctattcatcaaaagagaaaaagctggtcacccttgaggaatatgtgtccagaatgcctgaagatcaaaaatacatttattatgcatcaggcgactcgtatgaccgaatagaaaagcttcctcaaaccgagctggtttcggaaaaagggtatgaaatcctttatttcaccgaggatatcgatgagttcgccatcaaaatgctggcgaattatcaagagaaagaattcaagtcggtatcaagcggcgacctcggaatcgaaaacgatgacgagcaaaaccaatctgacggggatgacagccaatacaaagacctgtttgaagagatgaaaaagactctggatggaaaagtgaaaagcgtgagggcgtcaaaacggctgaagactcactccgtatgccttgccgctgacggagaagtcaccatcgagatggagaaaatcttaaacgcaatgcctgacaatcagcatgtaaaggctgataaagtattggaaatcaatacaaaccacgaagtcttcaaaacattgcagaatgcttttgacaacgacaaggataaattcaaactgtacaccggcctcctgtacaaccaggcgctgctgatagaaggcttgccaattgaagaccccgtcgaatttacgaacgatatatgcaaagtaatggcataa

SEQ ID NO: 2 - protein, 626 aa, Bacillus licheniformis DSM 13
MAKREFKAESKRLLDIMINSIYSQKEVFLRELISNASDAIDKIYYKALTDDSLTFNKDDYYIKISADKENRTLTIADTGIGMTKEELEEHLGTIAKSGSLAFKQENELKDGHDIIGQFGVGFYAAFMVADTVTVITKAHGSDEAHQWESAGADGYTIEPAAKESAGTDVILKLKENTDDENYDEYLDVHRLKAIIKTYSDFIRYPIKMDVAVNKPKEGAENEFEEVQEEQTVNSMVPIWRKNKSELKDEDYEAFYKEKHYGFDKPLAHIHTSVDGAVRYHAILFIPENIPFNYYTKEFEKGLELYSNGVLIMEKCPDLLPDHFSFVKGMVDSEDLSLNISREMLQHDRQLKLIAKNISKKIKNELKSLLKNDREKYESFYQSFGRQLKFGVYNDFGAHKDLLKDLLLFYSSKEKKLVTLEEYVSRMPEDQKYIYYASGDSYDRIEKLPQTELVSEKGYEILYFTEDIDEFAIKMLANYQEKEFKSVSSGDLGIENDDEQNQSDGDDSQYKDLFEEMKKTLDGKVKSVRASKRLKTHSVCLAADGEVTIEMEKILNAMPDNQHVKADKVLEINTNHEVFKTLQNAFDNDKDKFKLYTGLLYNQALLIEGLPIEDPVEFTNDICKVMA

SEQ ID NO: 3 - DNA, 1221 bp, Bacillus licheniformis DSM 13
  gene (1)..(1221): cypX; cytochrome P450-like enzyme; BLi03567
  CDS (1)..(1221)
atgaatcaatcgctaaaaacattcagcgtgttgtcagaacaatatcacgaaaatccgtatcagtatttctcataccttcgggaatctgatcccgtccattatgaagaatcgctggacagctattttatcagccgctaccaggatgtgcgccgcgtgctccagaatcaggacgtctttacaacgaaatcgcttgccaaacgggccgagcccgtcatgcgcggacctgtgctcgcccaaatgaaaggcaaagagcacacggcaaaaaggagaatcgttctgcgccgctttatcggggaatcccttgatcacctcacaccgctcatcaaagaaaatgcacaaaggcttttggccccgcacgtggagaaagggcggatcgaccttgtcaatgatttcggcaaaacattcgccgtttgcgtgacgatggacattttaggtttggacaagaacgaccaccaaacggtgagaaactggcacagcggcgtcgccgatttcattaccagtttgaatcaggcgcctgaggaccgggagcattccctcaaatgcagtgaacagctcgctgagtatttgaatccgattatcgaggaaaggcgcaaaaatcccggacatgatttaatatccatcctttgcacttccgaatacgagggagtggcgatgtctgaccgggacatacgcgcgctgattctcaatatattgcttgccgccacagaaccggcggacaagacgctcgcattaatgatttatcatttgcttcatcatcccgatcagatgaatgatgtgctggaagaccgcacgcttctccctcaggcaatcgcagagacgctgcgctacaagccgccggtgcagctcatcccgcgccagctttcacaagacgcggagattggcggagtcgagctaaaagaagggacgactgtattttgcatgataggcgcggcaaatcgcgatcctgaagcgttcgaggatcccgacaagttcaacattcaccgcagcgacctggaagtcaaaagcgcattcagcggcgcagccaggcatctcgcattcggttcaggcgtccacaactgtgtaggagcggggtttgcgaagaccgaaatcgagcttgtcgcaaatatcgtccttgatcagctgaaaaatatccggctggaggaagacttcatttaccgcgagacagggctttacacacgcggacccgtctcgctcaacatccggtttgatgccaaacattaa

SEQ ID NO: 4 - protein, 406 aa, Bacillus licheniformis DSM 13
MNQSLKTFSVLSEQYHENPYQYFSYLRESDPVHYEESLDSYFISRYQDVRRVLQNQDVFTTKSLAKRAEPVMRGPVLAQMKGKEHTAKRRIVLRRFIGESLDHLTPLIKENAQRLLAPHVEKGRIDLVNDFGKTFAVCVTMDILGLDKNDHQTVRNWHSGVADFITSLNQAPEDREHSLKCSEQLAEYLNPIIEERRKNPGHDLISILCTSEYEGVAMSDRDIRALILNILLAATEPADKTLALMIYHLLHHPDQMNDVLEDRTLLPQAIAETLRYKPPVQLIPRQLSQDAEIGGVELKEGTTVFCMIGAANRDPEAFEDPDKFNIHRSDLEVKSAFSGAARHLAFGSGVHNCVGAGFAKTEIELVANIVLDQLKNIRLEEDFIYRETGLYTRGPVSLNIRFDAKH

SEQ ID NO: 5 - DNA, 1068 bp, Bacillus licheniformis DSM 13
  gene (1)..(1068): ctaC; cytochrome caa3 Oxidase (subunit II); BLi01706
  CDS (1)..(1068)
atgttaaagaaatggcgtctgatgtcgctgttcactattttggcgcttgttttagcagggtgcggagaaccgtttatatcaacgctgcagccggcgggcgaggtggctgatcagcagtatttcctaatgattctcagcacgctgatcatggttgttgttgtagtagtggtaagtttcattttcgtatttgtcatcgtcaagtacagaagatctaagctcggtgatgaccacatcccgaaacaggttgaaggaaaccgcaatttagaaattttatggactgttattccgattatcctccttcttatcctggttgttcctgttgtcgcacagacctttgatcttgctgatacgaaaccgatggataagaaaaacagggatccggagaagacgctcgtcgtcaacgtgcgggcgaacctttactggtgggaattcgaatatcctgactacggcgtgattacgagccaggagctggtcgtgccgaccgatgaacgcatttacttcaagctgaaagcttccgatgtcaaacattcattctggattccttctgtcgggggcaaggttgatacgaacacagacaatgaaaacaagttctacttaacgattgattcaaaacgggcggaaaaagccggagactatttctacgggaaatgcgccgagctctgcggaccttcccacgctttaatggactttaaagttaaagcggtttcgaaggatgagttcaagaaatggatgagcgatatggaaaacaataaagcggaagcgaaaaccgatctcgctgttgaaggagaaaaggtgttcaaagagaaaaactgcctgagctgtcatgcgaccgattcaaaagaccaaagggcggaagcggccagaacggctccgaacctcgcgacttttggagacaggtcgacgatcgccggcgtgaaggaattcaacgatgaaaacttgaaagcgtggctcaaggatcctgaaagcataaaaccgggcaacaaaatgaccggcacataccctgatttgagcgacaaggaacttgatgcactgactgaatatttaaaggggttaaaaaccgaagactaa

SEQ ID NO: 6 - protein, 355 aa, Bacillus licheniformis DSM 13
MLKKWRLMSLFTILALVLAGCGEPFISTLQPAGEVADQQYFLMILSTLIMVVVVVVVSFIFVFVIVKYRRSKLGDDHIPKQVEGNRNLEILWTVIPIILLLILVVPVVAQTFDLADTKPMDKKNRDPEKTLVVNVRANLYWWEFEYPDYGVITSQELVVPTDERIYFKLKASDVKHSFWIPSVGGKVDTNTDNENKFYLTIDSKRAEKAGDYFYGKCAELCGPSHALMDFKVKAVSKDEFKKWMSDMENNKAEAKTDLAVEGEKVFKEKNCLSCHATDSKDQRAEAARTAPNLATFGDRSTIAGVKEFNDENLKAWLKDPESIKPGNKMTGTYPDLSDKELDALTEYLKGLKTED

SEQ ID NO: 7 - DNA, 321 bp, Bacillus licheniformis DSM 13
  gene (1)..(321): nasE; assimilatory nitrite reductase (subunit); BLi00485
  CDS (1)..(321)
atggtgaacaagaacttaacaaaggtgtttgtttcgaagatcggggatttgccgaatcagcttgggaagacagtgttcatcaaggacaaggaaatcgcggtattcaagctttcaaacggcagcgtccgcgcgattgaaaaccgctgcccccacaaagggggcgtgcttgccgaaggaatggtaagcggcgaattcgtcttctgtccgatgcacgactggaagatcagccttgaagacggcaaagtccaagagcctgacacggggtgtgtgcagacatacgaaacggtcgttgaaggcgaagatttgtatatcgtttattaa

SEQ ID NO: 8 - protein, 106 aa, Bacillus licheniformis DSM 13
MVNKNLTKVFVSKIGDLPNQLGKTVFIKDKEIAVFKLSNGSVRAIENRCPHKGGVLAEGMVSGEFVFCPMHDWKISLEDGKVQEPDTGCVQTYETVVEGEDLYIVY

SEQ ID NO: 9 - DNA, 1065 bp, Bacillus licheniformis DSM 13
  gene (1)..(1065): yrbE; similar to dehydrogenase; BLi00809
  misc_feature (1)..(3): First codon translated as Met.
  CDS (1)..(1065)
gtgacggttcattttggtttaatcggctgcggctatatgtcaagaaaacatcttcaagcactggccgagtgcgatgatgcaaagttgtcggccgtcagtgatttgcaggaagaaagaatgaaggaagcggaagaatactatgcttccctcgccggtgaggaaagccgaatgacccgctatccgcagtatcaagcgctgctttcagatcctaaaattgaagcggtcattattgcggcggtttcgggactgcacgccgaaatggccaaacatgcgctgctggcaggcaagcacgtcatcgtcgaaaaaccgatgaccttgtcattacgggatgccgatgagcttatagaactggcggagcagaacgggctgaagctcatggtctgccaccagatgcgccaccggccgatcatgaaaaaactgaaggaaacgattgaggaaggaaagctgggaaagatctacttgggcacggtatcgctccgcttaaacaggtcccccgcttattatgaagcggcatcctggcggggaagctgggaaaaagacggcgggatgctggtcaatcaagggatacatctgatcgatctgctgcaatggtttttaggtgatgtaaaaggcgtttacggagatatgctgggcggctccggagctaaagaaacggaagatgttgctgtcggtattctgacgtttgccagtcaggcgaaaggtgtagtagaagcgaatatcgtcacacagccaaacaatatcggaagctctctctcgatttttggagaaaatggaacgatttcattggaaggtccggcattaaaccggatcagccgctggtacatccacggggagaaggcggatgctgaagaagcggcaaagctgattgacatcggcggcgaacaaaccgcaatgtatcaaaactttataagcgccctcaagcgccaagacacgctattggttgacggctatgaaggaaaaaaggcgattgaaaccatctttggaatctatcaatctgtgctgcagaagcaaccggtgcagctgcctctttcttcatttcaaacaactgatatgaaaaataaatag

SEQ ID NO: 10 - protein, 354 aa, Bacillus licheniformis DSM 13
  misc_feature (1)..(3): First codon translated as Met.
VTVHFGLIGCGYMSRKHLQALAECDDAKLSAVSDLQEERMKEAEEYYASLAGEESRMTRYPQYQALLSDPKIEAVIIAAVSGLHAEMAKHALLAGKHVIVEKPMTLSLRDADELIELAEQNGLKLMVCHQMRHRPIMKKLKETIEEGKLGKIYLGTVSLRLNRSPAYYEAASWRGSWEKDGGMLVNQGIHLIDLLQWFLGDVKGVYGDMLGGSGAKETEDVAVGILTFASQAKGVVEANIVTQPNNIGSSLSIFGENGTISLEGPALNRISRWYIHGEKADAEEAAKLIDIGGEQTAMYQNFISALKRQDTLLVDGYEGKKAIETIFGIYQSVLQKQPVQLPLSSFQTTDMKNK

SEQ ID NO: 11 - DNA, 4338 bp, Bacillus licheniformis DSM 13
  gene (1)..(4338): yfkN; similar to 2′,3′-cyclic-nucleotide 2′-phosphodiesterase; BL i00814
  CDS (1)..(4338)
atggtgggaatacagaaaagacggttttcaaggaaaaacattctccgtattctgctaacctcagtgatgatactcagtttactcatgccaaacacccagacatatgcagaagaacaaaacgaacacccgctgatcagcctcagcatcttgggtacgacagatatccacgcccatatgatggattatgactactatgccgacaaagaaacggctgaattcggactggcacgaacggctcagctgatcgaaaagtaccggagccggaatcctaacacgattcttgtcgacaacggcgacttaattcaaggaaaccctctaggggaatacatcttcaaacatgaacgtgaatcgattatctccggaacgaaaacacacccgatcattgatgtcatgaacaaactgcaatatgacgccggcacgctcggaaaccacgaattcaactacggccttgaattccttgacggcacgataaaaggggccgaatttcccatcgtcaatgccaatattaaaacaccggacggaaagaacaaatatacgccatatcatattgaagaccggacgctgacggatgaaagcggccgaaagcaccagcttaaagtcggctatatcggcttcgttccgcctcagattttaacatgggataaaaagcacctggacggcaaaatcacagtgcaggacatcaccgaatcagcaaaagaaacgattccaaaaatgaaacaggaaggcgccgacgtgatcatcgcgatcgcccactccggaattgaaaatggcgaacaggcggcgggagccgaaaacgccgtatacgacctggctctcaagacggaaggaatagatgcgatcatctccggacatcagcacggtctgttccccggcaccgactatcccggtaacggcgtgatcgacaaccaaaaaggaacgatcaacggcattcctgtcgtcatgccgaagaactggggcagccatttaggaattatcgatatgcagcttgaactggacggcggcaaatggaaagtgaaagatgccagcgcaaaaacggaagcgattgccggaaatgtgacgtcgcgaaacgaaaccgtcgtttccgcgattaaagatacgcacgaaaaaacgctcgaatacgtccgcgcgccggttggccaaacagaagctgatattaacagctttttcgcgcaggtaaaagacgatccttcaatccagattgtcacagatgcacaaaaatggtatgcggaagatgtcatgaagggaacggaatacgaaaacctgccgattttatcagcgggcgcgccgttcaaagcaggaggacgcaacggtgctgattattatacaaacattaaagcaggaaaccttgcgatcaaaaacatcggcgatttgtatttgtacgacaatacgctctatatcgtcaaactgacgggaagcgaagtgaaggactggatcgaaatgtcggccgggcagttcaatcagatcgatccttcaaaatcgggtgaacaggcgctcctcaaccccgatttcaggtcgtataatttcgatgtcattgacggtgtgacctaccagatcgatgtcacacagcctgcaaaatacaatgcctcaggcggggtgaaaaatccgcaggcgaaccgcgtcaaacagttgtcttacaacggaaaacccgtccgcgacgaccaggaattccttgtcgtcacaaataattaccgcgcctccgggggcggcggattcccgcatctgactcaggacaaagtcgtttatgcggcggctgatgaaaacaggcaggtgctcatgaactacattatggagaagaaaacggtcaatcccgccgccgacaacaactggtcgatcgctccgattaaaggagccgacgtgacattcgagtcttccctccttgccaaaccgtttgctgaaaaagcgaatgacatcgattttgtcagggaatccgaaaacaaaggatatggcgtctataagctgaactttgacggttcagagcctccagttgatgatgaaaactggacattaaccgtcatgcacacgaacgacacgcatgcccaccttgatggagcggcaaggcgcgctgccaaagtgaaagaaatccgcagtgaaacagaaaacaatgtgctgctcgatgccggcgacgtcttttccggcgatctgtattttacaaaatggaacggactcgccgatttgaaactgatgaacatgatgggctatgacgccatgaccttcggcaaccatgaatttgataaagggcctaaagtgctcgcagactttctcagcggaaactatacagccgtcgacccgttaaacagacaccagctggaaaagcccgattttccgatcgtcagctccaatgtggatgtatcaaaagagccgaagctgcgaagctttcagaaagagcctgcagtcttagcagcgggaaaaaagaaagaaagcggcatctatccttatattctgcttgatgtaaacggcgaacaagtcggcgtattcggcttgacgacggaagatacgtcatatacatcaagccccggtccgaacattgcgtttcatgacgcttatcagtcagcagagaaaacggtgaaggaactgaaagaaaaagaaaaggtcaacaaaatcatcgccctcacccacctcggctacaaccgggatctggagcttgccaagaaagtgaagggcatcgatttgattatcggcgggcatacgcatacacttgtcgatcacttgaaagtggtggagaacgaagaaccgacaatcgtcgctcaagtgaaagactacggacagtttttgggaagggtcgatgtggtgtttgatgagaacggcgtcgttcaaacggaccaatcaaggttcaatttgatcgcgatcgacgacaaagtggacgaagaccctcctgggaaagaagtgcttgacggctttaaagaagatattgacgaactgaaaaagaaaaaagtaggctatgccgatgtcccgctggacggaaaacgcgagcatgtccgctcaaaagaaaccaacctcggcaattttatcgcggacggcatgctgaagaaagcaaaacaagccggcggagctgacatcgccatcacaaacggcggcggaatcagaagcggtattgccaaaggcgaaatcacactcggcgatgtgctgacggtcatgccgttcggcaacaccctttatgccgctgatttgacgggggcgcaaatcaaagaagcgcttgagcacggggtgagccaagtcgaagaaggcggaggcgcgtttccgcatgtttcgggtctatccttcacctttacactgagcaagccaaaaggtgagcgaatccttgatgtgaacctagtcaatgacaaaggggacgctgttccgctcgatttggacaaaacctaccgtgtcgctacaaataacttcatgggcggcggcggagacggctattccgtttttaaagaagcgaaaaacggtgtcgatctcggctttaccgattacgacatttttacagagcagcttgccttatacgattctgtttcaccgaacatcgagaacagaatcacagaagtgttccttcccgaccgtcaggcagacggctcttggacgcttgcggaaaagccgttcgaaacatacgcgaaaaacgcaaatcaggcgattgcctatttccctgacacagaaaaggaacacatcatgctgaatatgacaaaagcacagactgaccttgtcaaagaaagaaccggtgatccaagcatcaccctgaaaaacgatcgcattgcgatgcagcttccagccgttaatctggcaagcgaaaagacaaaaatcagcattaaaatcgccaaggatatcaaacaggcggtcacaaaggtatatgatttcaacattcagcagaatggtaaaacggtcagcacatttaaagagcctgtcggactgttgttccaagtcgatgctcctgataaacatttgagcgtctattatgtcgaccgcaaaaacaacagcttcacaaaagtaacaaacggctatacgaaaggccgtaccgttgcaggctatgtcgaccatttcagcgagtataccgtgatgaatgacaatgatgccgggtcgccttcccttccgacgggttccggcgatgatgacactccggatggcggcagcggcggtgaaccccctgcttcgcccggcgccggcagcggcggagacggatcaggcggtcatgacagcggcggagcagatgacggaaatgaaccttccgatggttcaaacggcggttttttgcctgacaccgcttcaaacgcctatcattggctgatgctcggcctgctgttgcttatatccggaagcggcctgtacctttaccaaaggaagcggcgcaagctccagatctaa

SEQ ID NO: 12 - protein, 1445 aa, Bacillus licheniformis DSM 13
MVGIQKRRFSRKNILRILLTSVMILSLLMPNTQTYAEEQNEHPLISLSILGTTDIHAHMMDYDYYADKETAEFGLARTAQLIEKYRSRNPNTILVDNGDLIQGNPLGEYIFKHERESIISGTKTHPIIDVMNKLQYDAGTLGNHEFNYGLEFLDGTIKGAEFPIVNANIKTPDGKNKYTPYHIEDRTLTDESGRKHQLKVGYIGFVPPQILTWDKKHLDGKITVQDITESAKETIPKMKQEGADVIIAIAHSGIENGEQAAGAENAVYDLALKTEGIDAIISGHQHGLFPGTDYPGNGVIDNQKGTINGIPVVMPKNWGSHLGIIDMQLELDGGKWKVKDASAKTEAIAGNVTSRNETVVSAIKDTHEKTLEYVRAPVGQTEADINSFFAQVKDDPSIQIVTDAQKWYAEDVMKGTEYENLPILSAGAPFKAGGRNGADYYTNIKAGNLAIKNIGDLYLYDNTLYIVKLTGSEVKDWIEMSAGQFNQIDPSKSGEQALLNPDFRSYNFDVIDGVTYQIDVTQPAKYNASGGVKNPQANRVKQLSYNGKPVRDDQEFLVVTNNYRASGGGGFPHLTQDKVVYAAADENRQVLMNYIMEKKTVNPAADNNWSIAPIKGADVTFESSLLAKPFAEKANDIDFVRESENKGYGVYKLNFDGSEPPVDDENWTLTVMHTNDTHAHLDGAARRAAKVKEIRSETENNVLLDAGDVFSGDLYFTKWNGLADLKLMNMMGYDAMTFGNHEFDKGPKVLADFLSGNYTAVDPLNRHQLEKPDFPIVSSNVDVSKEPKLRSFQKEPAVLAAGKKKESGIYPYILLDVNGEQVGVFGLTTEDTSYTSSPGPNIAFHDAYQSAEKTVKELKEKEKVNKIIALTHLGYNRDLELAKKVKGIDLIIGGHTHTLVDHLKVVENEEPTIVAQVKDYGQFLGRVDVVFDENGVVQTDQSRFNLIAIDDKVDEDPPGKEVLDGFKEDIDELKKKKVGYADVPLDGKREHVRSKETNLGNFIADGMLKKAKQAGGADIAITNGGGIRSGIAKGEITLGDVLTVMPFGNTLYAADLTGAQIKEALEHGVSQVEEGGGAFPHVSGLSFTFTLSKPKGERILDVNLVNDKGDAVPLDLDKTYRVATNNFMGGGGDGYSVFKEAKNGVDLGFTDYDIFTEQLALYDSVSPNIENRITEVFLPDRQADGSWTLAEKPFETYAKNANQAIAYFPDTEKEHIMLNMTKAQTDLVKERTGDPSITLKNDRIAMQLPAVNLASEKTKISIKIAKDIKQAVTKVYDFNIQQNGKTVSTFKEPVGLLFQVDAPDKHLSVYYVDRKNNSFTKVTNGYTKGRTVAGYVDHFSEYTVMNDNDAGSPSLPTGSGDDDTPDGGSGGEPPASPGAGSGGDGSGGHDSGGADDGNEPSDGSNGGFLPDTASNAYHWLMLGLLLLISGSGLYLYQRKRRKLQI

SEQ ID NO: 13 - DNA, 3621 bp, Bacillus licheniformis DSM 13
  gene (1)..(3621): yhcR; similar to 5′-nucleotidase; BLi00982
  CDS (1)..(3621)
atggtcaatgttgtaaagagccgttttatggctggtttgacaatcacgtttatgatgattgcgtcttttttgacgccgtttgcggacgtaacgcacgcttcagaaacgatttcagtagaagaagccattaagcataacgaagggcatgcagccgttgaaggctatgtcgtcgggcacacaatatcgagcggccattatcgtttcagcggaaatttcgctaacgattataacgtcgcaatcgctgataaaaaggaggaaacatcacctgagaacatactccccgtgcagatacccgcacctttcagagaccagttcggtttaaagacaaaccctcatctcatcggaaaaaaactcatcatcgaaggcgaacgcactacatattttaattccccgggtttgaaatctgttcaatccatcacattttccggagacgatgcaccgtctgcccagccggagaccaaaacgatagccgaagcgcgcaagcttttaaatgaaacggtcaaaatcaaaggaatcgtcactgccgatcaggcggctgtcggaggaggaaagctgtccacatttatccaggacgatacggcgggaatcaatatctattccgctgcgcaaggcgattttccggaactaaaagagggtatgtctgtttcggtcacggggaaaatcacgacatacaaaggcttgacagaaatcgtacccgcaccgtcaggcattgaagtaaccggagaaggggcggcactgcccgaccctgttccgctgactattaaagatgtgctcgatcagggaaccgcagctgaatacgaaggccggcttgtgaggatgaaaggctttattgaaacaaagccggatgcgccggcaggcggcggctataatatcacaatgatcgatgaaaactatcatccgctcacactaagggtgatggaacaaacgaatgccgccgcttcgatagaacaaggacaatggtacgagattaccggcatattaagccgctatgattcggtgcagcttctgccgagaaaacaggaggatcttcggaaattagacggcgccgcgcatcctccccccgctgctgaaggggaatacgaaagcgttgtggaacgagtcgtcgacggggatacgatccacttaaaagagcctgttctcggatctacaaaagtccgctatgtcaacatagatacagccgaaacttatcataagccgaaaaatgaattagatgaaaaccagctgcagcacggaaacaaggcgaaggaatatttgcagtcgatattggccccgggagacaaagtcaccgtgaaagtgggcaaggaagcaaaagacagctacggccgcctgcttgcccaagtcatcactgaagacggcgtcaatacgaatttgcagcttgtccaaaaggggatggctgtaacctacttcatttggccggttggaagtgatgaagactatcaattgtttcaaaacgccgtcaaaaaggcgaaaggcgataaaatcggcatttggaatcccgctgatccgctcctcgaaatgccgtttgaatttcgcgacagggagcaaggcaaagggctgacgcggcatgtcggagactctgcggaaaatacgtatgtcgcgcctgaaaagtggcgggagattccggtggaaagacgaattttctttgcgtcggctgaagaagcggaagctgcgggctattcgaaaaaagagcaaagcggcaatattccgctgcgcctgttaagcatgaatgatctgcacggaaagatcgatcagcaatatcagctcgatctaaacggcgatggccggacggatggaacattcggcaggatggattatgcagcatcattgatcaaagagaaaaaagccgagcaggaaaacacgctgatcgtccacgcaggcgatatgatcggaggaagctcgccggtttcttccctgcttcaggacgagccgacagttgaagtcatggaggaaatcggatttgatgtaggaaccgtcgggaaccatgagtttgatgaagggaccgaagagctgctgcgcatgttgaacggaggcgaacaccctgaaggcaaagggacaaaaggttacgacggacaggatttcccgcttgtctgcgccaattgcaagttgaaggcaagcggggacagctttcttcctccatatgaaattattgaagttgaaggagtgcctgtcgcttttatcggcgttgtgacacaggcggccgccaatatggtcatgccagaaggaattcgctcaattgaatttacagatgaagcaaaggccgtcaaccaggctgcaaaagaactgagagcgaaaggtatccgggcaatagcggtgctcgcccatatgacagcttcacaggacggcggggttgtgaccggcgaatcagccgaacttgctaaaaaagcagatcccgagatcgatgtcatctttgccggccacaatcatgaagtggtcaacggccaagtcaatgatattttaatcgttcaagcatacgaatacgggaaggcaatcggtgtcgtcgatcttgaaatcgaccgggaaacgaaagacatcgttcacaagaaagcggaaatcgaatatgtgaatcaggaaaatctcaagcccgatccggcggtcggtcagatactggataaatatgagcggatggttgcgccgatcatcagcgagaaagtcggtgaggctgcgcacgatatggccggcggctattcaaatgacggagacacccctctcgggaatctgattgctgacggtatgcggcaagcgatgaattcagatttcgcattaatgaacgggggcggcatccgccaagatttaaaaaaaggaacgattacatggggagacttgttcaatatccagcctttcggcaatgtactcgtccggcttgaaattaaaggcagggatttatacgatattatagacgcgcaaatatccccatcttacggcccggattactcgatcagcggcttcgcttatacatgggacccgaaaacaaataaaaccgcggctatttttcttgaagacggaacaccgattgatttagaccaaacatatacgctgaccgtaaacaactttatggcatctgcttcaggaagcaagtatttgccgatttccaggcttggaaaaaatccggtcacaggtccggaagatatcgaagccacagtcaaatttgtcaaaagctttaaagaaccgatcgcttatactgccgaagggcgaatcaaaaaattagcagaagatgattcgcgtccgggagaacagcccggacgcggggctgatccagatgacaaagaaaaacccggaagcggaggcgctgatccgaatgacgaagaaaaaccgtctggtgataagggcgatggggggaaacccggcaatgacaccggagaaagtccggttcatgacggtaccggcaatgcttcaaggagcggcgctattcctgttgcgggcatgacgaccggtgaaggatcagatgcgcctgcggagaagatggacggtctgtcaaatgatgcgtttctgccagacgagcgtgaagagcgcaagctacccgatacggcggcagcttacggcggtttccttttatacggcttcctgacgtttatgttcggagcgattctttatatgcggaaaaggggtattgcgagactatga

SEQ ID NO: 14 - protein, 1206 aa, Bacillus licheniformis DSM 13
MVNVVKSRFMAGLTITFMMIASFLTPFADVTHASETISVEEAIKHNEGHAAVEGYVVGHTISSGHYRFSGNFANDYNVAIADKKEETSPENILPVQIPAPFRDQFGLKTNPHLIGKKLIIEGERTTYFNSPGLKSVQSITFSGDDAPSAQPETKTIAEARKLLNETVKIKGIVTADQAAVGGGKLSTFIQDDTAGINIYSAAQGDFPELKEGMSVSVTGKITTYKGLTEIVPAPSGIEVTGEGAALPDPVPLTIKDVLDQGTAAEYEGRLVRMKGFIETKPDAPAGGGYNITMIDENYHPLTLRVMEQTNAAASIEQGQWYEITGILSRYDSVQLLPRKQEDLRKLDGAAHPPPAAEGEYESVVERVVDGDTIHLKEPVLGSTKVRYVNIDTAETYHKPKNELDENQLQHGNKAKEYLQSILAPGDKVTVKVGKEAKDSYGRLLAQVITEDGVNTNLQLVQKGMAVTYFIWPVGSDEDYQLFQNAVKKAKGDKIGIWNPADPLLEMPFEFRDREQGKGLTRHVGDSAENTYVAPEKWREIPVERRIFFASAEEAEAAGYSKKEQSGNIPLRLLSMNDLHGKIDQQYQLDLNGDGRTDGTFGRMDYAASLIKEKKAEQENTLIVHAGDMIGGSSPVSSLLQDEPTVEVMEEIGFDVGTVGNHEFDEGTEELLRMLNGGEHPEGKGTKGYDGQDFPLVCANCKLKASGDSFLPPYEIIEVEGVPVAFIGVVTQAAANMVMPEGIRSIEFTDEAKAVNQAAKELRAKGIRAIAVLAHMTASQDGGVVTGESAELAKKADPEIDVIFAGHNHEVVNGQVNDILIVQAYEYGKAIGVVDLEIDRETKDIVHKKAEIEYVNQENLKPDPAVGQILDKYERMVAPIISEKVGEAAHDMAGGYSNDGDTPLGNLIADGMRQAMNSDFALMNGGGIRQDLKKGTITWGDLFNIQPFGNVLVRLEIKGRDLYDIIDAQISPSYGPDYSISGFAYTWDPKTNKTAAIFLEDGTPIDLDQTYTLTVNNFMASASGSKYLPISRLGKNPVTGPEDIEATVKFVKSFKEPIAYTAEGRIKKLAEDDSRPGEQPGRGADPDDKEKPGSGGADPNDEEKPSGDKGDGGKPGNDTGESPVHDGTGNASRSGAIPVAGMTTGEGSDAPAEKMDGLSNDAFLPDEREERKLPDTAAAYGGFLLYGFLTFMFGAILYMRKRGIARL

SEQ ID NO: 15 - DNA, 813 bp, Bacillus licheniformis DSM 13
  gene (1)..(813): yurI; similar to ribonuclease; BLi03441
  CDS (1)..(813)
atgaaccgaaagtgtgtcattccgtttattttgatgttgtcggcgatgtgcgcgcccgcccaaaacgcagaggccttccagctgttttccttacccgtacaatcctcggctccggcagattactacgaacaggcgcagggcaaaacaggtgaagctttaaaacgagcgcttcacgacacaatcgacgatcatcgagagctgtcttacagcgaggtatgggaagcgctgaaagcaacggatgaagatccggccaatcgaaacaacgtgatactgttatattccagagagtcgcgctctaaacaggcgaacggggggcaaacgggcgattggaaccgcgagcacgtatgggcaaaatcgcacggtgatttcgggacaagcaaaggacccgggacagatttgcatcacctcaggcccagcgatgtgcaagttaacgccgcccgcggaaaccttgattttgacgaaggcggcagcccctatcccggagctcccggaaactactatgacggcgattcctgggaacctgacaaaagcatcaaaggcgatgtcgccagaatgattttctatatggcggtccgctatgaaggggacgacggtcatccggatcttgaaatgaacaatacaacgaacaacgggtctaaaccgtaccacggaaaaatgtcggttttattgaaatggcatcatgaagaccctgtcgatgccctcgaaagaaagcggaacgatatcatctatcagcaataccagcataaccgcaatccgttcattgatcaccctgaatgggcggaggatatttggggctcgggcgttttgaattga

SEQ ID NO: 16 - protein, 270 aa, Bacillus licheniformis DSM 13
MNRKCVIPFILMLSAMCAPAQNAEAFQLFSLPVQSSAPADYYEQAQGKTGEALKRALHDTIDDHRELSYSEVWEALKATDEDPANRNNVILLYSRESRSKQANGGQTGDWNREHVWAKSHGDFGTSKGPGTDLHHLRPSDVQVNAARGNLDFDEGGSPYPGAPGNYYDGDSWEPDKSIKGDVARMIFYMAVRYEGDDGHPDLEMNNTTNNGSKPYHGKMSVLLKWHHEDPVDALERKRNDIIYQQYQHNRNPFIDHPEWAEDIWGSGVLN

SEQ ID NO: 17 - DNA, 1476 bp, Bacillus licheniformis DSM 13
  gene (1)..(1476): homolog to dhaS; probable aldehyde dehydrogenase; BLi03994
  CDS (1)..(1476)
atgaatgttcaaactccatcggaaaaactcgtaaaaccttttgactgtcagcactacataaacggcaggtactttccttcagagaaaggcagcacgtttgacaacatcaatccggctacacaggaaattatcggaagcgttgcggaaggaggcaaaaaagaggtcgatcttgcagtcgcggcagccaggcgtgcattaaacggacgctggaaaaacatgaccgctgacgaaaggattcggatcatcagaaaggtcggcgacatcatacttgagcgtaaagacgaactggcgaggcttgagacattggatacgggaaaaccgctttcgctctcaagcactttggatattccacgtgccgctttcaactttcatttctttgcggattttatgagaggagccggaacggaagcttatcaaactgacgatcttgcaatcaattatgcgattcgccgtccggtcggcgtcgtcgggctgatcaatccgtggaacttgccgcttcttttgctgacgtggaaattggcgccttgcctggcggcgggaaatacagccgttatcaaaccggctgaactgacgccgatgacagccacccttctcggggaaatctgccatgatgcagggatgcctgacggagtcgttaatatcgtccacggatttggaccagattccgccggtgctgcgctttctgagcatcccgatgtcgatgcgatatctttcaccggagaaacgacaaccggcaaagtcattatggaagctgcttcgaaaacattaaagaaactttcctttgaattgggagggaagaacccgaacattatttttgccgatgccgacatggatgaagccgtcagcacctcattgaaatcgagtttcgtcaaccagggggaagtatgtatgagcggctcccgcatctatgtcgagagggaagcgtatgacgaatttctgaacaagttcgttgagaaaacgaaaaagctcaaagtcggcgatccttttgatccggacacgaatatcggcgccttgatttcttctgaacacacagagcgtgtgatgaactacatcgaattggccaaacgggaaggcggcaatattttaaccggaggaaaacgccctgaaggacaggaatccggctgctttcttgaaccgacgattatcaccggactttcacgcaattccaggctcatcaaagaagaaatcttcggtccggtcgtaacggtgattccgttcgacgatgaggaggaagtgattgcacaggctaacgacacacattacgggttaagcgcgaccttatggacgaacgacctccgccgggcgcaccgcgtagccgggcaaatcgaatcgggcatggtgtgggtcaacagctggtttctccgcgatcttcggacgccgtttggcggcatgaaacagagcggactcggacgagaaggaggcattcacagcctcgaattcttcagcgaattaaccaatatttgcatcaagctgtaa

SEQ ID NO: 18 - protein, 491 aa, Bacillus licheniformis DSM 13
MNVQTPSEKLVKPFDCQHYINGRYFPSEKGSTFDNINPATQEIIGSVAEGGKKEVDLAVAAARRALNGRWKNMTADERIRIIRKVGDIILERKDELARLETLDTGKPLSLSSTLDIPRAAFNFHFFADFMRGAGTEAYQTDDLAINYAIRRPVGVVGLINPWNLPLLLLTWKLAPCLAAGNTAVIKPAELTPMTATLLGEICHDAGMPDGVVNIVHGFGPDSAGAALSEHPDVDAISFTGETTTGKVIMEAASKTLKKLSFELGGKNPNIIFADADMDEAVSTSLKSSFVNQGEVCMSGSRIYVEREAYDEFLNKFVEKTKKLKVGDPFDPDTNIGALISSEHTERVMNYIELAKREGGNILTGGKRPEGQESGCFLEPTIITGLSRNSRLIKEEIFGPVVTVIPFDDEEEVIAQANDTHYGLSATLWTNDLRRAHRVAGQIESGMVWVNSWFLRDLRTPFGGMKQSGLGREGGIHSLEFFSELTNICIKL

SEQ ID NO: 19 - DNA, 1497 bp, Bacillus licheniformis DSM 13
  gene (1)..(1497): dhaS; aldehyde dehydrogenase; BLi02249
  misc_feature (1)..(3): First codon translated as Met.
  CDS (1)..(1497)
ttgacaaacatgagttcattaacgatgcaggtcagtaagaagctggagtcatttttgcagggaacaaagaagctctatattaacggggaatttgtagccagcgcctcaaacaaaacgtttgagaccccaaacccggcgactggtgaaacgctcgccacgttatatgaagcaggtcccgaggatgtggacaaagctgtaaaagctgcgcgggaagcctttgaccgcggtgagtggcggacgatgaaccctgccgaaagaagccggctgatgtacaaactcgccgatctgatggaggaacataaaacggaactggctcagcttgaaacgctggataacggaaagccgatcagcgaaacgaccgcaggcgatattccgctgtcaattgaacatatgcgctattatgcgggctgggcgacgaaaataaccggacagacaattcctgttaacggaccttattttaactatacacggcatgagcctgtcggagttgtcgggcaaattatcccttggaacttcccgcttctgatggctatgtggaaaatgggagccgcccttgcagccggatgtacggttgtcttaaaaccggctgaacaaacgccgctttccgctctgtatttagctgagctgatcgatgaggcaggcttcccgaaaggcgtcatcaacatcatccctggctttggggagacggccggagaagctctgaccgaccatgcccttgtcgacaaactggcattcacagggtcaactgaaatcggcaaaaagataatggagaaagcggccaaatcggtcaaacgtgttacccttgagctcggcggaaagtcgccgaatatcatcctccctgacgccgacctgaagaaagcgattccaggcgcactaaccggcgttatgttcaaccagggccaagtttgctgtgcgggatcgcgcgttttcatccataaagaccaatatgacaaagtcgttgatgaaatggccgcttattcaaaatcgctccgccaaggtgcgggccttcatgaagacacgcaaatgggaccgcttgtcagcaaagaacagcatgaacgcgtcctctcgtatattgaaaaaggacgccaggagggtgcgaaaatcgcggccggcggaacatgcccttatgaacagggatactttgtttctccgactgtattcacaaatgtcgaggatgagatgacaatagcaaaagaagaaatcttcggtcctgttttggcggcgattccttatgaaacggttgatgaagtcatcgaacgggccaatcgcagcgaatacggcctcgctgcgggcgtatggacggaaaatttgaaaaacgcccactatatcgccgaccgtctgcaagccggtaccgtttgggtcaactgctataatgcttttgacgccgcatccccattcggcggctacaaacagtctggactcggacgtgaaatgggctcatacgctctggataactatacagaggttaagagcgtctggattcatacaggcgaataa

SEQ ID NO: 20 - protein, 498 aa, Bacillus licheniformis DSM 13
  misc_feature (1)..(3): First codon translated as Met.
LTNMSSLTMQVSKKLESFLQGTKKLYINGEFVASASNKTFETPNPATGETLATLYEAGPEDVDKAVKAAREAFDRGEWRTMNPAERSRLMYKLADLMEEHKTELAQLETLDNGKPISETTAGDIPLSIEHMRYYAGWATKITGQTIPVNGPYFNYTRHEPVGVVGQIIPWNFPLLMAMWKMGAALAAGCTVVLKPAEQTPLSALYLAELIDEAGFPKGVINIIPGFGETAGEALTDHALVDKLAFTGSTEIGKKIMEKAAKSVKRVTLELGGKSPNIILPDADLKKAIPGALTGVMFNQGQVCCAGSRVFIHKDQYDKVVDEMAAYSKSLRQGAGLHEDTQMGPLVSKEQHERVLSYIEKGRQEGAKIAAGGTCPYEQGYFVSPTVFTNVEDEMTIAKEEIFGPVLAAIPYETVDEVIERANRSEYGLAAGVWTENLKNAHYIADRLQAGTVWVNCYNAFDAASPFGGYKQSGLGREMGSYALDNYTEVKSVWIHTGE

SEQ ID NO: 21 - DNA, 1662 bp, Bacillus licheniformis DSM 13
  gene (1)..(1662): phoB; alkaline phosphatase III; BLi02565
  CDS (1)..(1662)
atgggtttcttacgcaacagaatcgtcggcattacactggcaggcgcagtcgctttaggatcggcgggaacaggcagtgcggcaatggaaacagcgaaaaaaaaagaagagccgaaggtcaaaaacgtgatcatgatggtcatggatgggacaagctcttctgcaacaactttggcacgctggtataaaggcgaaccgctcgcaatggaccagatcgtcagcggcggtgtccggacgtattcggcagaatcggcgatcactgattctgcacctgcagcaactgcgatggcaacgggacgaaaatcaaattcttcctatatcggtgtgctgccttcaaaagtgaatacaccaggagtcgagaaggtccgtgaaaaagatcattaccggcctgtagcaaacgtgctggaaggcgcaaaacagaaggggaaagcaaccggcatcattgcaacatctgaaattcagcatgccacgcctgccggctattccggccatcataaagaccgcaataattttgaagtgctcggtgaacagcaggtttatcaaaacatggatgttgtccttggcggaggaaaagaatcgcttcagccgggtacaaaggataaaagccgcaaagacggtgaagaccttgtaaaagtgctcaaagataaaaagtacgatttcgttgaaacgaaagatcaactcctcaagtcaaaatcgaaaaaaatctggggcgccttctcacagcgcgatctcgcgtatgacatggaccgccgcgcaacgcatccagagcagccgactattgcagagatgacgaaaaaatcgattcagacgctttctaaagacaaggacggtttctttttattcgtggaaggaagcaagcctgactgggcagcgcatcaaaacgatccgatcggcatgatcagcgatgtattggcgtttgataacgcggtgaaagaagcgcttgatttcgctaaaaaagataaacatacaatggttattgccgtctcagaccatggcaacagcggcatctccatcggcaacagcaacacgacgaaaggctatgacacaacaccggtatcagcctatgtcgatccgctgaaaaaagctaaaatgacggtggaaggcgcaactggcaaattaaaagacgacttatcgaatatcgaagaagcggcgaagctttacggacttgatgatttaacgccggcagagaaggaacagctgaaaagcgtgaaaaacaaaaaagaagcggcttcattatttaccaagctgctcagcaaccgggccaacatcggattcacaacaggcggtcatacgggtgaagacgtcttcctgtattcgtatggtccagaaaagccgcagggtctcgtcgaaaacaccgatatcgccaaaacgatggcaaaggcaatgggcttcaacttagacaaattaacaaacgaattatttatcgaagcaaaacaggcatttaaaaaagccggagccagcgtaacgatcgatcaaaaagatccggctaacccagttcttgtcgcagcgcgcaaaaaagcaaaagccgaaatgcctgtgaacaaaaacattatcagaatcaacgggaaggaatacgaactagacagcgtgattgttgaaagcaatggatccttctacgtgcctaaagaagcagttcagctattcaaaaaatacgccagataa

SEQ ID NO: 22 - protein, 553 aa, Bacillus licheniformis DSM 13
MGFLRNRIVGITLAGAVALGSAGTGSAAMETAKKKEEPKVKNVIMMVMDGTSSSATTLARWYKGEPLAMDQIVSGGVRTYSAESAITDSAPAATAMATGRKSNSSYIGVLPSKVNTPGVEKVREKDHYRPVANVLEGAKQKGKATGIIATSEIQHATPAGYSGHHKDRNNFEVLGEQQVYQNMDVVLGGGKESLQPGTKDKSRKDGEDLVKVLKDKKYDFVETKDQLLKSKSKKIWGAFSQRDLAYDMDRRATHPEQPTIAEMTKKSIQTLSKDKDGFFLFVEGSKPDWAAHQNDPIGMISDVLAFDNAVKEALDFAKKDKHTMVIAVSDHGNSGISIGNSNTTKGYDTTPVSAYVDPLKKAKMTVEGATGKLKDDLSNIEEAAKLYGLDDLTPAEKEQLKSVKNKKEAASLFTKLLSNRANIGFTTGGHTGEDVFLYSYGPEKPQGLVENTDIAKTMAKAMGFNLDKLTNELFIEAKQAFKKAGASVTIDQKDPANPVLVAARKKAKAEMPVNKNIIRINGKEYELDSVIVESNGSFYVPKEAVQLFKKYAR

SEQ ID NO: 23 - DNA, 1722 bp, Bacillus licheniformis DSM 13
  gene (1)..(1722): phoD; phosphodiesterase/alkaline phosphatase; BLi00281
  CDS (1)..(1722)
atgaaaaaactgagcgaggaaagcctcaaggacaatacgtttgaccgccgccgctttattcaaggggccggcaaaatagccgggctttcgctcggacttgcgatcgcgcaatcgatgggggcaatggaagtcaatgcagcaccgaggttctccgaatatccgtttacacttggcgttgcatcgggagatccgctttctgacagcgtcgtattgtggacaaggctggcgcccgatccgctaaacgggggcgggatgccaaatgaagctgtatccgtgaaatgggagctcgcagaagacgaacgcttccgccgtgtcgtcaaacggggaaccgaaaaagcaacacctcaccttgcacattcggtacatgccgaggtatccgggctgaaaccgaatcatgtatactattaccgctttaaatgcggcaaccagctgagtcctgtcggcaagacaaagacgcttccggcgcccggggcagatgttgctaaattcacattcgcttttgcttcatgccagcagtacgaacacggctattataccgcctatcagcatatggcaaaggaaaagctcgatcttgtttttcatctcggcgactatatttatgagtacggtccgaatgaatatgtttcgaaaacaggaaatgtcagaacacacagcggtcctgaaatcatgtcactgctcgattaccgaaaccgtcatgcccaataccgttcagatgcaaacctgaaagctgcgcatgccgccttcccgtgggttgtcacatgggacgaccatgaagtggagaacaactatgcgaatgtcatcccggaaaaaggccagtcagttgaagcgtttattaaacggcgcgccgccgcataccaagcttattacgagcatatgccgctccgccgccaatccttgccgaacggtcctgatatgcaattgtaccggaatttttcctacggcaatttagctgacattagcgtgctggatacccgtcagtatcgcgatgaccaggctaacggcgatggcaataagccgccttctgatgaatcgaatgatccgaagcggacgctgcttggagcggagcaggaggcttggctgtttgacaatctgagtcgctccgaggcgcactggaacattatcgcacagcagattttctttgcgcagtggaactttgggacaagcgcggcgccgatttacagcatggattcgtgggacggatatcccgctcagcgcaagcgggtgatcgacttcatcaaatctcaaaaactgaacaacatcgtcgtccttaccggtgatgtgcatgccagctgggcaaataatctgctcgttgattttgacaatccgaaatcggacatctttggcgtggagttcgtcgggacgtcgattacatcgggaggaaacggcgctgataaaagagcggatacggataagattttatcgaacaatcctcatataaagtttttcaatgattaccgcggctatgtgcgctgtacggtcaccccggaagaatggcggacagattaccgcgtcgtcccgtatgtgacagagccgggtgcggcggtttcgacaagagcttcctatgtttatcataaagactacacagggctgaaacggaccgcttccaacattgtgcctggaggcgtgaagaagtcaaacgaagtagaagaagaccgtttcctcgcacatacaagggcccatcagaagcaagtgaagcaaaaggagaaaaaagtaacccagtaa

SEQ ID NO: 24 - protein, 573 aa, Bacillus licheniformis DSM 13
MKKLSEESLKDNTFDRRRFIQGAGKIAGLSLGLAIAQSMGAMEVNAAPRFSEYPFTLGVASGDPLSDSVVLWTRLAPDPLNGGGMPNEAVSVKWELAEDERFRRVVKRGTEKATPHLAHSVHAEVSGLKPNHVYYYRFKCGNQLSPVGKTKTLPAPGADVAKFTFAFASCQQYEHGYYTAYQHMAKEKLDLVFHLGDYIYEYGPNEYVSKTGNVRTHSGPEIMSLLDYRNRHAQYRSDANLKAAHAAFPWVVTWDDHEVENNYANVIPEKGQSVEAFIKRRAAAYQAYYEHMPLRRQSLPNGPDMQLYRNFSYGNLADISVLDTRQYRDDQANGDGNKPPSDESNDPKRTLLGAEQEAWLFDNLSRSEAHWNIIAQQIFFAQWNFGTSAAPIYSMDSWDGYPAQRKRVIDFIKSQKLNNIVVLTGDVHASWANNLLVDFDNPKSDIFGVEFVGTSITSGGNGADKRADTDKILSNNPHIKFFNDYRGYVRCTVTPEEWRTDYRVVPYVTEPGAAVSTRASYVYHKDYTGLKRTASNIVPGGVKKSNEVEEDRFLAHTRAHQKQVKQKEKKVTQ

SEQ ID NO: 25 - DNA, 735 bp, Bacillus licheniformis DSM 13
  gene (1)..(735): tatCD; component of the twin-arginine translocation pathway; BLi00283
  misc_feature (1)..(3): First codon translated as Met.
  CDS (1)..(735)
gtgaaaaaaagggaaacacatttgattgcccatcttgaagagctgcgccagcgaatcattaaaacgcttcttacgtttattctgtttctcatcaccggcttcttgtttgttcaggacatctatgactggctgatcagagaccttgatgaaaagcttgctgtactggggccgggggatattctttgggtttatatgatgattgcgggggtgctggcgatcgcagccaccataccggtggctgcttttcagacatggcggtttgtggcgcctgcgctgaccgaccacgaacggaggacggcattattttatattccggggctgttcattttttttattttggggattgtattcggttattttgtactctttccgctcgttcttcagtttttgatcggcctgtccgccggccattttcaaacgatgtttacggcggagcattattttcgctttatgatgaacctcacactgccattcgggtttttgtttgaaatgcccctcatcgtgatgtttttaacgagcctcgggattttaaatccggccaagctcagtaaaatgagaaaaatttcttattttgcgctgattgtcgtatcggttgtgatcacgccgcccgatttcatctcagatgtgctggtgatcgtcccgctccttgtattatacgaattgagcatctctttatcggcatttatctaccggaaaaaaaccgccgcacaacaaggggctgaggcccaaggctga

SEQ ID NO: 26 - protein, 244 aa, Bacillus licheniformis DSM 13
  misc_feature (1)..(3): First codon translated as Met.
VKKRETHLIAHLEELRQRIIKTLLTFILFLITGFLFVQDIYDWLIRDLDEKLAVLGPGDILWVYMMIAGVLAIAATIPVAAFQTWRFVAPALTDHERRTALFYIPGLFIFFILGIVFGYFVLFPLVLQFLIGLSAGHFQTMFTAEHYFRFMMNLTLPFGFLFEMPLIVMFLTSLGILNPAKLSKMRKISYFALIVVSVVITPPDFISDVLVIVPLLVLYELSISLSAFIYRKKTAAQQGAEAQG

SEQ ID NO: 27 - DNA, 762 bp, Bacillus licheniformis DSM 13
  gene (1)..(762): alsD; alpha-acetolactate decarboxylase; BLi03847
  CDS (1)..(762)
atgaaaagtgcaagcaaacaaaaaataattcagcccgttgataagaacctcgatcaagtctatcaggtctcaacgatggtatctttattggacggaatttacgacggggatttttatatgtccgaagcgaaggagcacggagacttcgggatcggaacgttcaaccggctcgacggcgagctgatcggttttgacggtgagttttaccgtctccgttccgatggaaaagcctacccagttcaaggaagcgattgttctccattttgctcgctggctttcttccggccggatatctatcacgaaatcaagcagcggatgccgcttgaggcgttcgaagaagaaatgaaacggatcatgccgagtgaaaacctgttttacgcgattcgcatggacggaacctttaagaaagtcaaaacgagaacagttgaacttcaggaaaaaccgtatgtgccgatggttgatgcggtaaaatcacagccgatctttgattttaatgatattacggggacgatcgtcggcttttggacaccgcaatatgccaacggaatcgcagtttccggcttccatcttcactttatagatgaagaccgcaatgtcggcggacacgttttcgattatgaaatcgaagaatgcacggtgcaaatttctcaaaaactcaatatgaacctcagattgccgaatacgcaagatttctttcaagcggatttcaataaacacgatcttgcagccggaattgaagcggccgaaggcaatcccgagtaa

SEQ ID NO: 28 - protein, 253 aa, Bacillus licheniformis DSM 13
MKSASKQKIIQPVDKNLDQVYQVSTMVSLLDGIYDGDFYMSEAKEHGDFGIGTFNRLDGELIGFDGEFYRLRSDGKAYPVQGSDCSPFCSLAFFRPDIYHEIKQRMPLEAFEEEMKRIMPSENLFYAIRMDGTFKKVKTRTVELQEKPYVPMVDAVKSQPIFDFNDITGTIVGFWTPQYANGIAVSGFHLHFIDEDRNVGGHVFDYEIEECTVQISQKLNMNLRLPNTQDFFQADFNKHDLAAGIEAAEGNPE

SEQ ID NO: 29 - DNA, 1719 bp, Bacillus licheniformis DSM 13
  gene (1)..(1719): alsS; alpha-acetolactate synthase; BLi03848
  misc_feature (1)..(3): First codon translated as Met.
  CDS (1)..(1719)
ttgaataatgtagccgctaaaaatgaaactcttactgtaagaggagcagagcttgtggtggatagtctcattcagcaaggtgtcactcatgttttcggtattccgggagcgaaaatcgatgcggtgtttgacgtattgaaagacaaggggcctgaattgatcgtttgccgtcacgagcagaatgcagcatttatggcggcggcagtcggacgattgactggaaagcccggtgtttgcctggtgacttcaggtccgggagcgtctaatttagcgaccggtcttgtaacagccaatacagaaggagatccggttgttgccctggcgggtgctgtaaaaagagcggatcgtctcaaaaaaactcatcaatcgatggataatgcggcgttgtttcagccgattacgaaatatagcgcagaagtggaagatgcgaacaacatacctgaggctgtaaccaatgcattcagagcggcggcttctggacaggctggcgcagcgtttctcagctttccgcaggacgttacggccggtccggcaactgccaagccggtgaaaaccatgccggcgccgaagctgggcgcggcttcggacgaacaaatcagcgcggccatcgccaaaattcacaatgcgaaccttcctgtcgtgcttgtcgggatgaaaggcggaagacctgaagcgattgaagcggttcggcgtctgctaaggaaagtgaaactgccgtttgttgaaacataccaagcagcgggtacgctgtctcacgatttggaagaccagtacttcggccggatcggactattccgcaatcagcccggagacatgctattggaaaaagcggatgtcgttttgacggtcggctatgatccgattgaatacgatccggtcttttggaatggaaaaggcgaacgaagcgtgattcatcttgacgaaatacaagccgatatcgatcatgactatcagcccgaaatcgagttgatcggcgatatcgcagagacgttaaaccatattgaacatgattctctgccggtttccatcgacgagtcctttgcacctgtccttgattacttgaagaaagcactggaggagcaaagcgaacctcctaaagaaacaaaaactgatcttgttcatccgctgcaaatcgttcgcgatttgcgtgaactgctgagcgatgacataacggtgacttgcgacatcggctcccatgcgatttggatgtctagatatttccgcacctaccgtccgcatggactgctgatttccaacggcatgcagacgcttggggtggctttgccgtgggcgattgcagcaacgctggtcaatccgggacagaaagtcgtgtctgtttccggggacgggggcttcctgttctccgcgatggagcttgagacagcggtcagattaaaagcgccgatcgttcacattgtctggaatgacagcacatacgatatggttgcgttccagcaggagatgaaatacaagcggacgtccggagtcgacttcggcggaatcgatattgtaaaatatgctgaaagctttggtgcaaaaggtttaagggtcaattcacctgacgaattggcagaggtgcttaaagctggtcttgatgcagaggggcctgttgtcattgatattccggtcgattacagcgacaacattcatttggccgatcagcgttttccaaaaaaatttgaggaacattttaataaagaagcctctaagcaatcttga

SEQ ID NO: 30 - protein, 572 aa, Bacillus licheniformis DSM 13
  misc_feature (1)..(3): First codon translated as Met.
LNNVAAKNETLTVRGAELVVDSLIQQGVTHVFGIPGAKIDAVFDVLKDKGPELIVCRHEQNAAFMAAAVGRLTGKPGVCLVTSGPGASNLATGLVTANTEGDPVVALAGAVKRADRLKKTHQSMDNAALFQPITKYSAEVEDANNIPEAVTNAFRAAASGQAGAAFLSFPQDVTAGPATAKPVKTMPAPKLGAASDEQISAAIAKIHNANLPVVLVGMKGGRPEAIEAVRRLLRKVKLPFVETYQAAGTLSHDLEDQYFGRIGLFRNQPGDMLLEKADVVLTVGYDPIEYDPVFWNGKGERSVIHLDEIQADIDHDYQPEIELIGDIAETLNHIEHDSLPVSIDESFAPVLDYLKKALEEQSEPPKETKTDLVHPLQIVRDLRELLSDDITVTCDIGSHAIWMSRYFRTYRPHGLLISNGMQTLGVALPWAIAATLVNPGQKVVSVSGDGGFLFSAMELETAVRLKAPIVHIVWNDSTYDMVAFQQEMKYKRTSGVDFGGIDIVKYAESFGAKGLRVNSPDELAEVLKAGLDAEGPVVIDIPVDYSDNIHLADQRFPKKFEEHFNKEASKQS

SEQ ID NO: 31 - DNA, 786 bp, Bacillus licheniformis DSM 13
  gene (1)..(786): gdh; glucose 1-dehydrogenase; BLi2566
  CDS (1)..(786)
atgtatccaagtttagagggaaaaacggtcgttatcacaggggcgggtacaggccttggaaaagcgatggcactgcgcttcggtgctgaaaaagccaatgtagtgatcaattatttcaaggaagaagaaaatcctgaagagacggtggagaaaatagaaagcagcggagcgcgcgcgattgccgttcaaggagatgtctcaaaagaagatgatgtaaaagcgctgatcagacaggctgttgattcattcggttccgttgatgtgatggtgaacaacgcaggggtggaaaatgaagtaccttctgaggatttggctctagaggactggaaccgcgtcatatcaaccaatttgacggggatgtttctaggatgccgggaagcgatcggctatatgctcgaccataatattaaaggctcggtcatcaatatgtccagcgtccatcagcaaatcccgtggccgcattttgtccattatgcggcaagcaagggcggggcaaaactgctgacggaaacccttgcccttgaatatgccccgaaaggaatccgggtaaacgcgatcggccccggtgcaatcgatacgccgattaacgccgaaaaattcgctgatcccgaattgaaaaaaggcgttattgaattgattcctatagggtatatcggaaagccggaggaagtcgcagcctgcgcggcctggctagcatccgaagaagcaagctatgtgacgggcctgacgctgtatgtagacggcgggatgacaaaatacccgggattccaagcgggaaaagggtga

SEQ ID NO: 32 - protein, 261 aa, Bacillus licheniformis DSM 13
MYPSLEGKTVVITGAGTGLGKAMALRFGAEKANVVINYFKEEENPEETVEKIESSGARAIAVQGDVSKEDDVKALIRQAVDSFGSVDVMVNNAGVENEVPSEDLALEDWNRVISTNLTGMFLGCREAIGYMLDHNIKGSVINMSSVHQQIPWPHFVHYAASKGGAKLLTETLALEYAPKGIRVNAIGPGAIDTPINAEKFADPELKKGVIELIPIGYIGKPEEVAACAAWLASEEASYVTGLTLYVDGGMTKYPGFQAGKG

SEQ ID NO: 33 - DNA, 1146 bp, Bacillus licheniformis DSM 13
  gene (1)..(1146): phy; phytase; BLi00448
  CDS (1)..(1146)
atgaacttttacaaaacgctcgccttatcaactcttgcggcatccttattgtctccctcatggagcattctcccccgtgccgaagcttcagcttataaggacttctcggtgactgccgatgcagagacagagccggtggatacccctgacgacgcggcagatgacccggcgatttgggttcatccgaagcagcctgaaaaaagccggctgatcacaacaaataaaaaatctggcttgatcgtttacgatttaaacggaaaacagctggcagcctatccgtttggcaaattaaacaacgtcgatctccgctacaattttccgctcgatggcaaaaaaattgatattgccggggcctcaaaccggtcagacggcaaaaacacggttgaaatatacgcctttgacggcgaaaaaaacaagctgaaaaacatcgtcaatcctcaaaaacctattcaaaccgatattgaggaggtgtatggtttcagcctgtatcacagccagaaaaccggcaagttctacgccatggtgaccggaaagaacggagaattcgagcaatacgaactgtttgacaacggaaaaggacaagtcgaaggcaaaaaggtccgctcattcaaaatgagctctcaaacagaagggcttgcagcagatgatgaatacggcaaaatgtacatcgctgaagaagacgctgcgatttggtctttcagcgccgagccaaacggcggagataaaggaaaaattgtcgatcgcgcaggcggaccgcatttaaccgctgatattgaagggctgacgatttactacggagaagacggcgaaggatatttgatcgcgtccagtcagggcgataaccgttatgccatctatgaccggcgcgggaaaaacgactatgtcgccgatttttcaattgatgacggtaaagaaatcgacgggacaagcgataccgatggaatcgacgtcatcggcttcggcctcggcaaaaaatatccatacggcatctttgtcgcccaagacggcgaaaatacggaaaatggacagccagccaatcagaacttcaaaattgtctcctgggaaaaaattgctgacgcgctggacgacaagcctgatatcgatgatcaggtcaatccccgaaaactgaaaaaacgagccaaataa

SEQ ID NO: 34 - protein, 381 aa, Bacillus licheniformis DSM 13
MNFYKTLALSTLAASLLSPSWSILPRAEASAYKDFSVTADAETEPVDTPDDAADDPAIWVHPKQPEKSRLITTNKKSGLIVYDLNGKQLAAYPFGKLNNVDLRYNFPLDGKKIDIAGASNRSDGKNTVEIYAFDGEKNKLKNIVNPQKPIQTDIEEVYGFSLYHSQKTGKFYAMVTGKNGEFEQYELFDNGKGQVEGKKVRSFKMSSQTEGLAADDEYGKMYIAEEDAAIWSFSAEPNGGDKGKIVDRAGGPHLTADIEGLTIYYGEDGEGYLIASSQGDNRYAIYDRRGKNDYVADFSIDDGKEIDGTSDTDGIDVIGFGLGKKYPYGIFVAQDGENTENGQPANQNFKIVSWEKIADALDDKPDIDDQVNPRKLKKRAK

SEQ ID NO: 35 - DNA, 354 bp, Bacillus licheniformis DSM 13
  gene (1)..(354): spoIIAA; anti-sigma F factor antagonist (stage II sporulation protein AA); BLi02497
  CDS (1)..(354)
atgagcctcggaatcgatattcacgtcaaagaatccgtattatgcattcggttgacaggtgaactcgatcaccatacagcagaaaccttgagaaaacaagtcagtgaccatctggaacaaaccgacattcgccatattgtcatgaatcttgcagacctttcgtttatggacagttcagggcttggcgtcgtgctcggcagatataaggagattaagcagctcggcggagaaatgatcgtctgcgccatttcccctgctgtcaaacgtttatttgatatgtccgggctgtttaaaatcattcgccttgagcaatcagagcagcgtgcacttgaaacgttgggggtggcgtcatga

SEQ ID NO: 36 - protein, 117 aa, Bacillus licheniformis DSM 13
MSLGIDIHVKESVLCIRLTGELDHHTAETLRKQVSDHLEQTDIRHIVMNLADLSFMDSSGLGVVLGRYKEIKQLGGEMIVCAISPAVKRLFDMSGLFKIIRLEQSEQRALETLGVAS

SEQ ID NO: 37 - DNA, 441 bp, Bacillus licheniformis DSM 13
  gene (1)..(441): spoIIAB; anti-sigma F factor (stage II sporulation protein AB); B Li02496
  CDS (1)..(441)
atgaaaaatgaaatgaacattcagtttacagcgctcagccaaaatgaatcgtttgcacgggtgacagtcgctgcttttatcgctcagcttgacccgacgatggatgaactgaccgaaattaaaacggtcgtatccgaagcggtcacaaacgcgatcattcacggttatgaaaactcagggcagggaaacgtatatatttccgtcactctcgaggaccatattgtctatttaacgatccgcgacgaaggagtcggcatccctaatcttgaagaagcgcgccagcccctgttcacgacaaagcctgaactcgagcggtcgggaatgggctttacgatcatggaaaatttcatggatgatatttcgatcgactcctcacctgagatgggaaccacaatacacttaacaaagcacttatcaaaaagcaaagcgctttgcaattaa

SEQ ID NO: 38 - protein, 146 aa, Bacillus licheniformis DSM 13
MKNEMNIQFTALSQNESFARVTVAAFIAQLDPTMDELTEIKTVVSEAVTNAIIHGYENSGQGNVYISVTLEDHIVYLTIRDEGVGIPNLEEARQPLFTTKPELERSGMGFTIMENFMDDISIDSSPEMGTTIHLTKHLSKSKALCN

SEQ ID NO: 39 - DNA, 549 bp, Bacillus licheniformis DSM 13
  gene (1)..(549): cotE; spore coat protein (outer); BLi01927
  CDS (1)..(549)
atgtctgaatacagggaaattatcactaaagcggtggtcgcgaaaggccggaaattcacccagtccactcatacaatctccccttcgcaaaagccaaccagtattttaggcggttggattatcaatcataagtatgacgctgaaaaaatcggaaaaaccgtggaaattgaagggacatatgatatcaacgtctggtattcatatgcggacaacaccaaaaccgaagttgtgacagaacgcgtttcctatgttgatgtcattaagctccggtatcgcgacaaaaattacttagatgatgaacacgaagtcattgcgaaagtgcttcagcagccgaactgccttgaagtgaccatttctccgaacggaaacaaagtggttgtacaggcggaaagagaatttttagcagaggtcgtcggcgagacgaaggtcgtcgttgaggtcaattctgattggacggaaagcgatgaagaagaagcgtgggaagaagagcttgatgaagaacttgaggatatcaatcccgagtttttggttggagatccagaagaataa

SEQ ID NO: 40 - protein, 182 aa, Bacillus licheniformis DSM 13
MSEYREIITKAVVAKGRKFTQSTHTISPSQKPTSILGGWIINHKYDAEKIGKTVEIEGTYDINVWYSYADNTKTEVVTERVSYVDVIKLRYRDKNYLDDEHEVIAKVLQQPNCLEVTISPNGNKVVVQAEREFLAEVVGETKVVVEVNSDWTESDEEEAWEEELDEELEDINPEFLVGDPEE

SEQ ID NO: 41 - DNA, 930 bp, Bacillus licheniformis DSM 13
  gene (1)..(930): spoIIGA; protease (processing of pro-sigma-E to active sigma-E); BLi01749
  misc_feature (1)..(3): First codon translated as Met.
  CDS (1)..(930)
gtggaaatctatttagatgcgatatggctgttaaacttttgttttgacttgctgcttttaatgatgaccgcatttattttaaagcgaagggttaaaaagcggaggctgatcctaggggcatttgtcgcgtcaagcatcgttctgtttatgtttacacctttttcaccgtacgtccttcatcctgccggcaaactgtcgttttcggttgtgatcgttcttgtggcatttggttttaagcggttccggttttttttgcagaatttgttttctttttattttgccacttttttaatgggaggagggattatcggagcgcattctttgcttgaaacggattcgatcatggaaaacggcgtctttatgacgaattggtccggttttggagaccccgtcagctggctgtttgtctgtgtgggttttgcggctgtatggctgttttcaaaaaagcgttttgaagatgctgaagcgaagaaaattcaatacgaagaacgcgtccgcctagaggcctgcattggtgaacatacgcttcatttcaccggattgattgactccggaaaccagctctacgatccaatcacaaaaacgcccgtcatgatcgtcaatattgaaaaattgaaagttgtattgggagaagaggcaagtgtgaccatcaaggaaatgagcccgcttgatgccgtcgggaaactggatgaagcactgccgtatatcgggcggatccgcctgattccgtaccgcggggtcggccatcagcatcagtttctgctctgcttaaagccggatcatgtgctcgtttgtacggaaagagaagtgattgaagcgccgaaatgcctgattggcatcagcacatcaccgctttccgctgacggcgaatttgacgccatcgtccatccgaaaatgctggccggaaacccggtcaaacacgtttcttaa

SEQ ID NO: 42 - protein, 309 aa, Bacillus licheniformis DSM 13
  misc_feature (1)..(3): First codon translated as Met.
VEIYLDAIWLLNFCFDLLLLMMTAFILKRRVKKRRLILGAFVASSIVLFMFTPFSPYVLHPAGKLSFSVVIVLVAFGFKRFRFFLQNLFSFYFATFLMGGGIIGAHSLLETDSIMENGVFMTNWSGFGDPVSWLFVCVGFAAVWLFSKKRFEDAEAKKIQYEERVRLEACIGEHTLHFTGLIDSGNQLYDPITKTPVMIVNIEKLKVVLGEEASVTIKEMSPLDAVGKLDEALPYIGRIRLIPYRGVGHQHQFLLCLKPDHVLVCTEREVIEAPKCLIGISTSPLSADGEFDAIVHPKMLAGNPVKHVS

SEQ ID NO: 43 - DNA, 876 bp, Bacillus licheniformis DSM 13
  gene (1)..(876): spoIIQ; required for completion of engulfment; BLi03892
  CDS (1)..(876)
atgaaagaggaagaaaagaatcgtacttccaaaatcacaaagctgcaacaattttttcgtaaacgctgggtatttccggccatctatttgacaagtgccgtcgttgtattaaccgccgttctatggtatcaatcggcttctaacaacgatgtaaaagaccagcttgcagacgatggcaagaaatcagcctatgataaccgggatgatgcggtagaagtaggcaaaccagtcgaaaatgtcgcaatgccggttgctgattctgaaaatgtttccgtcgttaaaaagttttttgaaactgacgcaactaaagaagagaaagaagcagcacttgtaaactataataacacgtacagcatgagcaaaggtatcgacttggctgagaaagacggaaaaacatttgatgtttccgcatctctaagcggtacggtcatcaaagctgcaaaagaccctgtactgggctacgttgttgaagttgaacatgaagatggtttatcaactgtgtatcagtctctttctgaagtaagcgtcaaacaaggtgacaagattgaacaaaatcaagtcatcggaaaagcaggcaaaaacctttacaatgaagaaggcggaaaccatgtgcattttgaaatccgcaaagacggtgttgcgctaaacccgctgaacttcatggacaagccggtctccagcattgaaaaagcaatggaggaacaagcgtctgaagtgaaagaacctgctcagccttctgttgaagaaaagtcaaaaacagaagacaaagcgaaagatcaaacagatggaaaagacgacaaaaccaagcgggaagattcgtctgaagggtcagaaaatcaagacggaacccagtctgacgattcaagccagtcataa

SEQ ID NO: 44 - protein, 291 aa, Bacillus licheniformis DSM 13
MKEEEKNRTSKITKLQQFFRKRWVFPAIYLTSAVVVLTAVLWYQSASNNDVKDQLADDGKKSAYDNRDDAVEVGKPVENVAMPVADSENVSVVKKFFETDATKEEKEAALVNYNNTYSMSKGIDLAEKDGKTFDVSASLSGTVIKAAKDPVLGYVVEVEHEDGLSTVYQSLSEVSVKQGDKIEQNQVIGKAGKNLYNEEGGNHVHFEIRKDGVALNPLNFMDKPVSSIEKAMEEQASEVKEPAQPSVEEKSKTEDKAKDQTDGKDDKTKREDSSEGSENQDGTQSDDSSQS

SEQ ID NO: 45 - DNA, 1374 bp, Bacillus licheniformis DSM 13
  gene (1)..(1374): spoVID, required for assembly of the spore coat; BLi02941
  misc_feature (1)..(3): First codon translated as Met.
  CDS (1)..(1374)
ttgccgcaaaacaatcgtttgcaattttctgtagaagagtcaatctgttttcaaaaaggacaggaagtatccgaattattatcgatttcattggatcctgacattaccgttcaggaagtaaatgattatgtatccatacgagggtcattagagctgacaggcgaatacaacatagatcaaacccgggagtatgcagagctgcctgcgacaagccgatttgtagaagatgtaaagctgaaaggggacggcagcgcagagctgacgcattgtttcccagtggatatcaccatcccgaaagacaaagtcaatcatttaaacgacgtatttgtttttattgacgctttcgattatcagctgacagatgcgaggatgctgacgattcaggctgatttggcgattgaaggcctcttgaatgtgagcggtgaagcgggtgaagaagaaccgcgcactatgcctgcggccgtccatccggaagaggagctcgaacctgcctacagatcaccttcaaacgacgaagatcagggtgaagagaaagaatatttgatccagcttgacagaccttacgaagagcaggacgaagaacaggcggaagaacatgataccggtgaggaaacggttccgatttaccagtcgtttctcggaaacgacacagaggaagctaaaccgttttttacagcgtctttgtcggcggcagagcgtacgaagcgcgaaatagaaaatcaaaaagaagcctctcttgaacagccggaagaagaatataagctgaaaagagagaaagtggaagaggaaccggaagaatatgagctgaaaagagagaaagtggaagaggaaccggaagaatatgagctgaaaagagaagaagcggaagaagagccggagctgtcgcacagctcttatcaacctcacgaggaactgaaagagaacccgttctacagtgttcctcctcttctgaaggaagaccagaatgacagggagcctgaggcttttgaggttgaggtgacacaggaagcagaagcgattgatgaagaagaggaagccgggcatacgattgaaatcccggaatattcgtttcatgagcagacggagcccgaagaagaaagagatgaaatgcaggcagcggatgaacaggaagtgtcagcaaaggaaaacgacaacgcactctatttgacaaagctgtttacaaagcagggagaggaggagtttactcgaatgaggatgtgcatcgttcagcaaaatgatacgattgatcttctgtgcgagcgctatgatattaacgtccagcagctcatccggatgaattccctttcccttgacgaggaattaaaagagggacagatcctttatataccggattatcaaaacagccatgcctaa

SEQ ID NO: 46 - protein, 457 aa, Bacillus licheniformis DSM 13
  misc_feature (1)..(3): First codon translated as Met.
LPQNNRLQFSVEESICFQKGQEVSELLSISLDPDITVQEVNDYVSIRGSLELTGEYNIDQTREYAELPATSRFVEDVKLKGDGSAELTHCFPVDITIPKDKVNHLNDVFVFIDAFDYQLTDARMLTIQADLAIEGLLNVSGEAGEEEPRTMPAAVHPEEELEPAYRSPSNDEDQGEEKEYLIQLDRPYEEQDEEQAEEHDTGEETVPIYQSFLGNDTEEAKPFFTASLSAAERTKREIENQKEASLEQPEEEYKLKREKVEEEPEEYELKREKVEEEPEEYELKREEAEEEPELSHSSYQPHEELKENPFYSVPPLLKEDQNDREPEAFEVEVTQEAEAIDEEEEAGHTIEIPEYSFHEQTEPEEERDEMQAADEQEVSAKENDNALYLTKLFTKQGEEEFTRMRMCIVQQNDTIDLLCERYDINVQQLIRMNSLSLDEELKEGQILYIPDYQNSHA

SEQ ID NO: 47 - DNA, 918 bp, Bacillus licheniformis DSM 13
  gene (1)..(918): pstS; phosphate ABS transporter (binding protein); BLi02676
  misc_feature (1)..(3): First codon translated as Met.
  CDS (1)..(918)
gtgaaaccatttaaaaaaatcacactgatgttcatcatgtccgttttagttgtcttcgcagcagcttgtggaagcagcagtacaaacaacaatgcagacgaaggcaaacaaagcgataaaacagagaataaggcttcaggttctttgacgatttcgggttcttcggcgatgcagcctcttgtcttagcagcggctgaaaaattcatggatgaaaatcctgatgccgatattcaagttcaggctggaggttctggtacaggcctttcccaagtggctgaaggcacagtccaaatcggaaactccgatgtctttgccgaagaaaaagaaggcattcctgccaaagaacttaccgatcatcaagttgcggttgtcggaatggcagctgccgttaaccctgaagtaggcatcgatgacatttcgttagaagatctcaaaaaggtattcacaggtaaaatcaaaaactggaaagaactaggcgggaaagatcaaaagatcacccttgtcaaccgtcctgattcttcaggaacccgcgcgacttttgtcaaatatgcacttgatggagagactccagctgaaggaatcacggaagattcttcaaacacagtgaaaaagttaatcgctgaaacgcctggtgcaatcggatatcttgcgttctcgtatatcaaggatgatactgtcactccgctttcaatcgacggcgttaagccgacagccgaaaatgtacaaaaaggcaaattcccgatctgggcttaccagcattcttatacgaaaggcgaacctgaaggcttggcgaaagaattcctcgactatttgatgagcgatgacattcaaaaatccatcgtgacggagcagggctacattccagtaacagacatggaagtaaaacgcgacgcggatggaaagcaaactgataaataa

SEQ ID NO: 48 - protein, 305 aa, Bacillus licheniformis DSM 13
  misc_feature (1)..(3): First codon translated as Met.
VKPFKKITLMFIMSVLVVFAAACGSSSTNNNADEGKQSDKTENKASGSLTISGSSAMQPLVLAAAEKFMDENPDADIQVQAGGSGTGLSQVAEGTVQIGNSDVFAEEKEGIPAKELTDHQVAVVGMAAAVNPEVGIDDISLEDLKKVFTGKIKNWKELGGKDQKITLVNRPDSSGTRATFVKYALDGETPAEGITEDSSNTVKKLIAETPGAIGYLAFSYIKDDTVTPLSIDGVKPTAENVQKGKFPIWAYQHSYTKGEPEGLAKEFLDYLMSDDIQKSIVTEQGYIPVTDMEVKRDADGKQTDK

SEQ ID NO: 49 - DNA, 1377 bp, Bacillus licheniformis DSM 13
  gene (1)..(1377): putative benzoate transport protein; BLi03990
  CDS (1)..(1377)
atgaaaaatcgttcagttcatccagcggaagtcatggcggccagcaaatttaacaaagtgcacctgactgttttcctgtggtgttttttcgccattgcctttgacggttatgatattgcaatgtacggcgtcagcctgccatggctgatggaggaatggagcctgaccgccatccaagccggagcgatcgggagctatacattgatcggcatgatgctcggcgcgcttattttttcgccgatcgcagatcaatacggccgcaaaaaggtgttgggcttctgtatctttctgttcagcctgtttactgcagcagccggtctgatcgattctccgcttttgtttacgatcatgcggtttctcgctgcgcttggaatgggcgggttgatgccgaatgccatttcgctgatgacagagtattcgccgaaaaaaaacagagcggtgattgtagccgctatgtattgcggctattctgcaggcggtgtgctggcttccctcgtcggcatgttcgccgtgcccgtgacaagctggagagtgctctatctgatcggagccgtgccgctcttcgtgctcccgttctttttcagcaggtttcccgaatctctgtcattttacgttttgaagaaaaagacgaaagcgctggctgagattttcaatcaggtgcatccggcgggacgctacagggaagacgacaattatcaaatctcggtgattgcgaaagaaaagaaaggctttccggtaaaaaaactgtttggacaaaaacgtgcagccagcacattcgcattctggctttccgttttcagctgccttctcatggtttacggcttaaatacatggcttccgaaaatgatgcaggcgtcaggctacgggctgtcgtcaagcttgtcattcaacttggcgctttgctgcggacaagccgcaggagcggtattcggcggatatttggcagataaagccggccataaaaatgtgcttgcggctatgtatgtcatcggagccttttgttttgcagcatttggaatgacgatgaaccccgttcttctgtacttgctgattgccttaggcggggcatgcaccgtcggagcgcaaaatatcgccaatccttatatttctgagtattatccgaaagaaatcagagcgaccggcatcggctgggcgctcggggtcggcagactcggagcgatcattgcgccttcgctgtttgcattgatccttgcttccggcatggagccgaagcaggctttcatggtgttcgcgatcccgagtgtttttgctgcactcggcatcatgctcgtccaagagaaacacgcttcatttgattttgtctctaagtcggaaagcgccgggtatacattagaagccacatcaaaacatggataa

SEQ ID NO: 50 - protein, 458 aa, Bacillus licheniformis DSM 13
MKNRSVHPAEVMAASKFNKVHLTVFLWCFFAIAFDGYDIAMYGVSLPWLMEEWSLTAIQAGAIGSYTLIGMMLGALIFSPIADQYGRKKVLGFCIFLFSLFTAAAGLIDSPLLFTIMRFLAALGMGGLMPNAISLMTEYSPKKNRAVIVAAMYCGYSAGGVLASLVGMFAVPVTSWRVLYLIGAVPLFVLPFFFSRFPESLSFYVLKKKTKALAEIFNQVHPAGRYREDDNYQISVIAKEKKGFPVKKLFGQKRAASTFAFWLSVFSCLLMVYGLNTWLPKMMQASGYGLSSSLSFNLALCCGQAAGAVFGGYLADKAGHKNVLAAMYVIGAFCFAAFGMTMNPVLLYLLIALGGACTVGAQNIANPYISEYYPKEIRATGIGWALGVGRLGAIIAPSLFALILASGMEPKQAFMVFAIPSVFAALGIMLVQEKHASFDFVSKSESAGYTLEATSKHG

SEQ ID NO: 51 - DNA, 1227 bp, Bacillus licheniformis DSM 13
  gene (1)..(1227): yvmA; similar to multidrug transporter; BLi03565
  CDS (1)..(1227)
atgagaccaatgaaatcaaatgcttctttcattatgtattttgtgtgtatcagcgcttttttcgcatcgttgaatcaaaatatttattcacctatcattcctttaatcagagactcgtttcacgtttctgtttcgatggtaaacctgtccgtctcgctgttcatcttcattacagctgcgatgcagatcattgtcggctcgattgttgattttaaaggagcgagatttgtaatgatggcgagcatgatcgtaacggtcatcgccacaatcggctgcgcagcctcccgggactttggcctgtttctttttttcagggtgctgcaggctttggggactgctgcgcttccattaattgcggcaaccacaatcggatctctgtttaagggaacgaagcggggcagtgcgatgggaacttatcaaatgctgctttcccttgcaccggcagcggcgcctattctcggcggttttatcggggagcgctataattatccgggtattttttggtttttgacagtattgtctgttcttttgcttttcggcaatttgttttgctttccgcgcagccattcggagaaaaagaccaatctcagcgcgggcagcctgctgtcgcattataaggacatattcaaaaacagggcgggcaatgccgttttaatattaagctaccttattttctttatttatttttcaatgatcgtgtatttgccgattttgctgaccgatcactatcaccttgatctgcagatcgtcggcttgctgtatttgccgatagcggtcagcacgattgccggcagcgtgctgtttaagtggatacaaaagagattatcgctcagacgcctgtttttgatcggcaatctgaccgccgcagggagcgtcgtctgttttgccttttcccatttgttttcgcttttcgccatgtcggcggctctggttcttttcggcgttacgatggggctgattccgccgctcttctctacgatgatggcgaatgaatttgaggagaaccgcggcagtgcgatgggcatgtttaattttatcaggtatacgggaatggcctgcggtcctgttatatcaggcatgttattggggatgtcatcttctgttttcgttttcggctgtttcggtatcgtttatgccgcagccgcccttttgatgaccgcggtcatgcggaaagcagctcacacaaagacgctgcaagaaccaaacaaaaaagctgccaggtcttaa

SEQ ID NO: 52 - protein, 408 aa, Bacillus licheniformis DSM 13
MRPMKSNASFIMYFVCISAFFASLNQNIYSPIIPLIRDSFHVSVSMVNLSVSLFIFITAAMQIIVGSIVDFKGARFVMMASMIVTVIATIGCAASRDFGLFLFFRVLQALGTAALPLIAATTIGSLFKGTKRGSAMGTYQMLLSLAPAAAPILGGFIGERYNYPGIFWFLTVLSVLLLFGNLFCFPRSHSEKKTNLSAGSLLSHYKDIFKNRAGNAVLILSYLIFFIYFSMIVYLPILLTDHYHLDLQIVGLLYLPIAVSTIAGSVLFKWIQKRLSLRRLFLIGNLTAAGSVVCFAFSHLFSLFAMSAALVLFGVTMGLIPPLFSTMMANEFEENRGSAMGMFNFIRYTGMACGPVISGMLLGMSSSVFVFGCFGIVYAAAALLMTAVMRKAAHTKTLQEPNKKAARS

SEQ ID NO: 53 - DNA, 783 bp, Bacillus licheniformis DSM 13
  gene (1)..(783): putative acetoin reductase; BLi02066
  CDS (1)..(783)
atgagtaaagtatctggaaaaattgcttttgttactggcggcggtcaaggaattggagaagcaatctgcaaacgattggcagaggacggattcgcagttgcagttgccgattataatgtagaaactgcaacacaagttgctgaggacatcaataagcttaacggcaaagcaattgcggttaaagtggatgttgctgatcgcgatgatgtttttaaagctgtcgatgaaacagtaaaacgtcttggcggtcttgatgtggtgattaataatgcgggtcttggaccaaccacccctattgaaagcattacatatgaagattatcggaaagtctatgatgttaacgttggcggtacttattggggaatacaagcagctgtaaaagcctttaaagaacttggacacggcgggaaaatcattaatgcatcttctcaagccggccaagtcggcaacccgggcttagcggtttacggaggaacaaagttcgctgttcgcgggattacccaaactgcggcaaaagatctagctgaattaggtattactgtaaacgccttttgtccgggtatcgttaaaactcctatgatgatggggattgcacagcaaaccgctgatgaagcaggcaagccgtttgaatggggcatggaacaattcgctaaaaatattgcattaaaacgcttatccgagccggaagatgtagcagcatgcgtttcttaccttgcagggccagattcagattatatgactggtcaagctcttatcattgatggcggaatggtatttaattaa

SEQ ID NO: 54 - protein, 260 aa, Bacillus licheniformis DSM 13
MSKVSGKIAFVTGGGQGIGEAICKRLAEDGFAVAVADYNVETATQVAEDINKLNGKAIAVKVDVADRDDVFKAVDETVKRLGGLDVVINNAGLGPTTPIESITYEDYRKVYDVNVGGTYWGIQAAVKAFKELGHGGKIINASSQAGQVGNPGLAVYGGTKFAVRGITQTAAKDLAELGITVNAFCPGIVKTPMMMGIAQQTADEAGKPFEWGMEQFAKNIALKRLSEPEDVAACVSYLAGPDSDYMTGQALIIDGGMVFN

SEQ ID NO: 55 - DNA, 852 bp, Bacillus licheniformis DSM 13
  gene (1)..(852): putative aromatic compounds specific dioxygenase; BLi03991
  CDS (1)..(852)
atgtcaatcgaattggcggcgcttgttccgcatacaccgaggatgtgttttgaagataaaacccctgacttccaaagagagctggtcaagggcatgaagaggctctccaggattattgaaagaattgagcccgatgccgttgtcctgatttcctgtcactggacgtcaagctttgaccatctgatcgatgcagctcccgaccatcaaggcgttttaacggctttggagtgtccgaatttaatttccgatgttccgtattcttaccccggggacactgaattggcgctgcaattggctgaagccggcagagaaggggggctttccgtcattcctgtcaatgatccagcttactgctgggattacgggacggtcgttccgctgcgctacctcgttccaaagggagatattccggttatcgatttatcggttacattggccgcgaacctggaagaaacgcatttgtggggccggcttgccggaagagtgctgaaagaaagcagaaagaaaaccgtgttcatcagcagcggggcgctaagccatcatttggtgagggggccggaatgtatgccgacattggccgaacaggccctcgatgcacaatttttgacctacttaacagacaacgatcttttatcagctcagcagatgctgcctcaatatgctaaaaacgccgggcttgaagccggcggccgccacattgcaatgctattgggcgtgctggaggacggatgtaaaagcacgttttacggatacggtcagtcttccgggagctccaatgtcgtcatggcattcgagccggcgggacaaaaagctcgcacgcacgctttcaatgaaaaagataccgtgcgttaa

SEQ ID NO: 56 - protein, 283 aa, Bacillus licheniformis DSM 13
MSIELAALVPHTPRMCFEDKTPDFQRELVKGMKRLSRIIERIEPDAVVLISCHWTSSFDHLIDAAPDHQGVLTALECPNLISDVPYSYPGDTELALQLAEAGREGGLSVIPVNDPAYCWDYGTVVPLRYLVPKGDIPVIDLSVTLAANLEETHLWGRLAGRVLKESRKKTVFISSGALSHHLVRGPECMPTLAEQALDAQFLTYLTDNDLLSAQQMLPQYAKNAGLEAGGRHIAMLLGVLEDGCKSTFYGYGQSSGSSNVVMAFEPAGQKARTHAFNEKDTVR

SEQ ID NO: 57 - DNA, 786 bp, Bacillus licheniformis DSM 13
  gene (1)..(786): putative decarboxylase/dehydratase; BLi03993
  CDS (1)..(786)
atgaataccgcagcattaaaagatacggcgcgactcttgtatcttgccgaaacagagaagcgtgaagttgaaagaatcacaaaggactacccggagctgaccgttgaagaggcgtatgcgatccaagaagagctgattcagcttaaattatgggatggcaatagcatcatcggtccgaaaatgggactgaccagccgggcgaaaatgaagcagatgaacgtcgaggagccgatctacggttatatttttgaagatatgatcgtgccaaacggcggaagcatccggatgaacgagctgattcacccgaaggttgaagcagagattgcttttgtgctgggggaggatattgaaggaccgggtgttacaaaggagcaggtccttgaagccgttgctgaattgattcccgttctagaagtgattgacagccgctatgaaaatttctcattcactttgcctgacgtcatcgcggacaatgcgtcatcttcaagagtggtgttaggtgaaaaagtgaagaagccggacgatttcaagctggatgaagcaagagtctctttaatgataaacggcgaggtcaaagagcggggcactggagcagcggttgtcgggcatcccgcaaattcggcggccatgctcgccaacatgctctcccggaaaaaacaaaagcttgcggctggcagcatcattttaacgggcggtgtaacgggagctgtcatgctgcagccgggcgaccgcgtttcagcacaagtagaagggctgggagatgtatcattttcggtcaagccataa

SEQ ID NO: 58 - protein, 261 aa, Bacillus licheniformis DSM 13
MNTAALKDTARLLYLAETEKREVERITKDYPELTVEEAYAIQEELIQLKLWDGNSIIGPKMGLTSRAKMKQMNVEEPIYGYIFEDMIVPNGGSIRMNELIHPKVEAEIAFVLGEDIEGPGVTKEQVLEAVAELIPVLEVIDSRYENFSFTLPDVIADNASSSRVVLGEKVKKPDDFKLDEARVSLMINGEVKERGTGAAVVGHPANSAAMLANMLSRKKQKLAAGSIILTGGVTGAVMLQPGDRVSAQVEGLGDVSFSVKP

SEQ ID NO: 59 - DNA, 402 bp, Bacillus licheniformis DSM 13
  gene (1)..(402): conserved hypothetical protein; BLi03193
  CDS (1)..(402)
atgtgcacgatttctgcctgcctgtctctcttgctgttcggctcgggaagtgttcgtacagcggggatggccagcgcccttgcacttttggtcagccaccttcaagtcatgctgatcaaaaagctgtatccgagaaagcggccgtatttaacgctaaaggaaactcaggttttgcaaaatccgctcaaggatcattcttttccatccggacatacgacagctgtattttctgttattacaccgttaatgatattctttccgatactcgcgcttttattaataccggttggcgtcagtgtggggctctccaggatttatctcggtcttcattatccatcggatgttctggcaggcacagccctcgggatttcagtcggaaccttgtcggcgatgatcttctaa

SEQ ID NO: 60 - protein, 133 aa, Bacillus licheniformis DSM 13
MCTISACLSLLLFGSGSVRTAGMASALALLVSHLQVMLIKKLYPRKRPYLTLKETQVLQNPLKDHSFPSGHTTAVFSVITPLMIFFPILALLLIPVGVSVGLSRIYLGLHYPSDVLAGTALGISVGTLSAMIF

SEQ ID NO: 61 - DNA, 1791 bp, Bacillus licheniformis DSM 13
  gene (1)..(1791): putative phosphatase; BLi02820
  misc_feature (1)..(3): First codon translated as Met.
  CDS (1)..(1791)
gtgagaatgaaacgattaaggatgaggaagcatttactgatagctgtctgtactttggcacttcttctaagttccccgattgtaagcgatgcgagcccggcaactaaaccaacaactgcagattcgccgcaatcttccggatttttcgtagaccattacaaaaataatatctctgccaatacgacggcggaatccaatcctgtcatcggcctgctttccgaatttaataaactttggactcccggaaagacatggaataccggtactaaactgaacagcagggtgctggatgccaacattcaaaaagtcgtggatattgctgaacgccgcacgatgcttgaggaaaatgctgcctattttgatgatcggcggagccagagctacagtataattgacggcctcggcaagcttgccggcgtctatcgaatgaacgcgggagcgacgacaacgatcaccagcattccggcagatgcctcgattagaaaatacaatgatgaaggaaccaattcgggcagcaccagctctgaacttggaaatgtcgtaagtttggtcaatactttacgcggcaactattcttcatcgaatccggctaaaagctatttcaactatccccgcccgtttcgctggaaagacaattcgatcattgttccaacgcttatccccgtcatcaatcctgatccgaacaaagacggaggttttccaagcggacacacgaacgccgcatatctcagcgcttttgctatggcctatgcgataccggagcgttatcaggagctgctgactcgcgcttcagaactcggtcataaccggattgttgccggtatgcattccccgctggacgtcatggggggacgagtaatggcaacagctttgtctgcagcaatcctgtctgaccccgcaaatgaaagattgaagaaaacggcttttgatgaagcccgccgtaaattattaacgcaaaccggtacagctgaagacagatacagcgattatgagaagaataaaaaacaatatacggaacgattgacatatggatttcgacaaatgaacaaaaccgccaaaccaatggcagttccaaagggagccgaagtcctgctggaaacacgttttccttaccttgacaaaaagcagcgccgttcggttttagccactaccggtcttccggccggctaccctgttcttgatgatcgagaaggatggggaaggcttaatctcttttccgcggcagatgggtatggggcttttaccaaaaatgttaccgtgaccatggattccgcaaaaggcggcttccatacagccgatcgctggcgcaacgacatctccggcaccggaaagctgaccaaaaaagggacaggcgctttgaagctggaaggggataatacatattccggcggtacacggattgatcaaggaacacttgagggcggttcggagacagctttcgggagaggtgatgttgcactaaacggaggcatccttaaggaagatgcgccgggaaaactgatcatcgaaggagactacaaacaatctgctaaaggaatacttgaacttcagctcagcggcaaaaaagatcagttgaaaattaagggaaaagcaagattgaaagggacattgcgtctcaattttacggacaattacgtaccggctgacggatcggcgatcataaccttccgcaagcgtcatggatcattttcttccgtcgagaccagtggattgccaagcaagtataaagtgaagatcatctataaatccaacagtattcagttgaaagttgagcaaaaggggagaagctga

SEQ ID NO: 62 - protein, 596 aa, Bacillus licheniformis DSM 13
  misc_feature (1)..(3): First codon translated as Met.
VRMKRLRMRKHLLIAVCTLALLLSSPIVSDASPATKPTTADSPQSSGFFVDHYKNNISANTTAESNPVIGLLSEFNKLWTPGKTWNTGTKLNSRVLDANIQKVVDIAERRTMLEENAAYFDDRRSQSYSIIDGLGKLAGVYRMNAGATTTITSIPADASIRKYNDEGTNSGSTSSELGNVVSLVNTLRGNYSSSNPAKSYFNYPRPFRWKDNSIIVPTLIPVINPDPNKDGGFPSGHTNAAYLSAFAMAYAIPERYQELLTRASELGHNRIVAGMHSPLDVMGGRVMATALSAAILSDPANERLKKTAFDEARRKLLTQTGTAEDRYSDYEKNKKQYTERLTYGFRQMNKTAKPMAVPKGAEVLLETRFPYLDKKQRRSVLATTGLPAGYPVLDDREGWGRLNLFSAADGYGAFTKNVTVTMDSAKGGFHTADRWRNDISGTGKLTKKGTGALKLEGDNTYSGGTRIDQGTLEGGSETAFGRGDVALNGGILKEDAPGKLIIEGDYKQSAKGILELQLSGKKDQLKIKGKARLKGTLRLNFTDNYVPADGSAIITFRKRHGSFSSVETSGLPSKYKVKIIYKSNSIQLKVEQKGRS

SEQ ID NO: 63 - DNA, 381 bp, Bacillus licheniformis DSM 13
  gene (1)..(381): hypothetical protein; BLi04308
  CDS (1)..(381)
atgaaaaatcatttgtatgagaaaaaaaagaggaaacctttgactcggacaattaaagcgacgctcgccgtgttgacaatgtccatcgctttggtgggaggcgctacggtgccttcatttgcatgggtgaatccgggttatcactaccagtacccatcggaaggtggtacatggaggtatggattcgtaaacgccgggctccgttcagagtacaaccacccgacaaaggtccacggctcgacagtgcaaaagctcatcgatggaaaagtggataaaacgaatagaagtattgatacggctgcgggccgctactctaatgcctatgtcggagccataaactcacctggtcttaagggtcgttactactatcgcaccaactaa

SEQ ID NO: 64 - protein, 126 aa, Bacillus licheniformis DSM 13
MKNHLYEKKKRKPLTRTIKATLAVLTMSIALVGGATVPSFAWVNPGYHYQYPSEGGTWRYGFVNAGLRSEYNHPTKVHGSTVQKLIDGKVDKTNRSIDTAAGRYSNAYVGAINSPGLKGRYYYRTN

SEQ ID NO: 65 - DNA, 675 bp, Bacillus licheniformis DSM 13
  gene (1)..(675): hypothetical protein; BLi00235
  CDS (1)..(675)
atgactcttttaaaaaaacaatatgaaatgtttaagtgtgtaacagatgagaattatctgaaagaatacaaaaaacagaatcactttgattgggttaattttgatttttttaggaagttacattttaataaacgggcgattaggtttaatcatgtatttcctaaaactaaagaaaaaacaagggaatgggaaaagtttgttaagaacttctgcaatcaacataattataccggtcaaagagttgaagaagatgcagactcttttgtaaactttctttcgaaaaaaaaaggaaacgaaagctatatagctcagctggctaaacacgaattaaatatatacctagttacaattggaaaagggaatggttttgtagatgcattctgttatccaattacatgttttaacataaccagcacagagactaaaaaacataattacttgtatataaaaggaaaacaaggggttagagtattcaagatagaacaaagtgtagcaatatggattgaaaattataagaatttattatgtattaaagaagcaactaatctaacacataattctttaaatactcaagaaaaaaatcagttaaaaattgctatagataactttaaaaaattaggtctgttggacatatggaagagagaaagtattaatgaaaagtctaaacaacattaa

SEQ ID NO: 66 - protein, 224 aa, Bacillus licheniformis DSM 13
MTLLKKQYEMFKCVTDENYLKEYKKQNHFDWVNFDFFRKLHFNKRAIRFNHVFPKTKEKTREWEKFVKNFCNQHNYTGQRVEEDADSFVNFLSKKKGNESYIAQLAKHELNIYLVTIGKGNGFVDAFCYPITCFNITSTETKKHNYLYIKGKQGVRVFKIEQSVAIWIENYKNLLCIKEATNLTHNSLNTQEKNQLKIAIDNFKKLGLLDIWKRESINEKSKQH

SEQ ID NO: 67 - DNA, 816 bp, Bacillus licheniformis DSM 13
  gene (1)..(816): yfkH; similar to proteins; BLi00820
  CDS (1)..(816)
atgagttttttgaaagagctgggctggcgttttttccttcatgaaggaccgagcaaatcggcggaacttgcctacttttttctattgtctctgtttccgtttatgattttcctattgacattgatcggctacctgcctgtcaaaacgggcgatgtcatcgggctgatcgaacagtatgccccagaagacacgatgtccgtcattgaagagaccctgaataccggaaacaggggcctgctttctttcgggatcattgcggcgctctggtctgcatcaaacggattgaacgcgattgtcagggcctttaaccatgcttatgaagtagaggaaaaccgctcttttatcatggttcggctgacatcgctccttttgaccgccgcgatggttttcacaatattggtcgcgcttcttctgcctgtattcgggagggccatcggccattttatcgccaatctggacggtacgccggagatgtttctgacggcatggtcagcgcttcgctggggaatcagcccgcttgtgctgctgatcgtctttacggctttgtacttttttgctccgaataaacggctgtcatttaaattcgtgcttccgggagctgttgcagcgaccgccggctggatcctcgtcagcgtcctgttctcatactatgtcagcgaatttgcaaactacagtgcgacatacggaagcctcggcggcatcatcgtcctgatgatctggttttatttaagcggaatgatgattattcttggaggggaaatcaatgcccttttacataagcgtaaaatcattcctgaagaaaatccgcggaattga

SEQ ID NO: 68 - protein, 271 aa, Bacillus licheniformis DSM 13
MSFLKELGWRFFLHEGPSKSAELAYFFLLSLFPFMIFLLTLIGYLPVKTGDVIGLIEQYAPEDTMSVIEETLNTGNRGLLSFGIIAALWSASNGLNAIVRAFNHAYEVEENRSFIMVRLTSLLLTAAMVFTILVALLLPVFGRAIGHFIANLDGTPEMFLTAWSALRWGISPLVLLIVFTALYFFAPNKRLSFKFVLPGAVAATAGWILVSVLFSYYVSEFANYSATYGSLGGIIVLMIWFYLSGMMIILGGEINALLHKRKIIPEENPRN

SEQ ID NO: 69 - DNA, 450 bp, Bacillus licheniformis DSM 13
  gene (1)..(450): yfmQ; unknown function; BLi00629
  misc_feature (1)..(3): First codon translated as Met.
  CDS (1)..(450)
gtgacatggctttttgtaacctttttggtgatcttcagtctttttaaaatattggtcacttgccttccgagcggtgtcgtagagtggctgaccggcaaatttcaagttcatgtgaagcttgaggaatcgaatgttcaagtgacattcggcggaaaacgtttagagggatctgacaagagtcgatttattcaaaactttaatgaagcggtatttatggagcggtactacatatacccgggagatgagccgctgtatgtcaatccgaaaaacgccgggacaccgtttgtgatcgaaacaactaaaggaaaaaacgctgtgaagctgtttgtatacagatacgatgatcatatagacgtcgttaaacaatataagaaaaagacggccgcttattgtctgcgttctgacagtctgcaaacatgcggggggatgaaaacggcggatttgatttaa

SEQ ID NO: 70 - protein, 149 aa, Bacillus licheniformis DSM 13
  misc_feature (1)..(3): First codon translated as Met.
VTWLFVTFLVIFSLFKILVTCLPSGVVEWLTGKFQVHVKLEESNVQVTFGGKRLEGSDKSRFIQNFNEAVFMERYYIYPGDEPLYVNPKNAGTPFVIETTKGKNAVKLFVYRYDDHIDVVKQYKKKTAAYCLRSDSLQTCGGMKTADLI

SEQ ID NO: 71 - DNA, 615 bp, Bacillus licheniformis DSM 13
  gene (1)..(615): yhbD; similar to proteins of B. subtilis; BLi00958
  CDS (1)..(615)
atggttcaggatgatttaatttcaaaaaaagagctgctggatttgacttcgatctcgtacggacagctctatagatggaaacggaaaaatttaatacctgaagagtggtttatccggaagtctacgttcaccggacaagaaacgttttttccgagggaagacattttaaaaagaattgaaaccattcaaagcatgaaagaaaacctttcccttgatgagatggcggaaatgttttcgcccgatttgaccgaactaaacgcgagccgcggggagctgctaaaaaaaggcatcgtttcagaagcggtcatggcgttttttgaagaatgcaccggaaaacgcgatcatccatttaatttgacagaagcacttgcggcttatgtgctggaaggcttgcttcaatccggcgacatcagcctggatgaagggaaaatggtgctggatgtcatcatgtcgaacgaggcggaaaacaagggaaggctgcttgtgctccggaagctcggcgttgcaacatgtctgattgccgagggcagcattgtgttggaaaagggagtcaaggtagccgctgacatcaatctggcggcgagcagtgaagaattgaaaacaaaattcatgtaa

SEQ ID NO: 72 - protein, 204 aa, Bacillus licheniformis DSM 13
MVQDDLISKKELLDLTSISYGQLYRWKRKNLIPEEWFIRKSTFTGQETFFPREDILKRIETIQSMKENLSLDEMAEMFSPDLTELNASRGELLKKGIVSEAVMAFFEECTGKRDHPFNLTEALAAYVLEGLLQSGDISLDEGKMVLDVIMSNEAENKGRLLVLRKLGVATCLIAEGSIVLEKGVKVAADINLAASSEELKTKFM

SEQ ID NO: 73 - DNA, 711 bp, Bacillus licheniformis DSM 13
  gene (1)..(711): yhbE; similar to proteins from B. subtilis; BLi00959
  CDS (1)..(711)
atggagaatttattgataaacggattcggcagttcaggcggaggcgcgtatcaaacggttgagttaaacggcaaaggtacgattaacggcgatattgactgcgaaaggttcagctgcagcggcaccgggactgtaaacggagacgtaaaagcggatgagctgagaatcagcggccatgcaaaactaaacggcgccatcgaggcttcaaaggttagaattgatggttctgcaaaagtggatggcgatgtcgaagcagaaaagcttaaaatcttaggctacgccggcattagcggacatgtgaaaggcgacgaagcgatcgttcgcggaaaaacgtcgatcggcaaagactgccaagtggaacatttttctgcagaaggctattttacaatcgacgggctgttaaacgcggagcaaatcgaaattaaggttcacggcggcgaaagtaaagtgaaagaaatcggctgccggaggatcgtctgcactgcacatcaatcgaagctgttctcgtttttatcggagcttatttctcctccaaggctgtctgccgagctgattgaaggggatcatatcgaacttgccaatacgaccgcaaaagttgtccgcggcaattcggttatcatcggcgaaaattgcgacatcggcctgattgagtataaggaagatatccgggtggccgaaacggcaaaagtcggagagaaaaggaaattatag

SEQ ID NO: 74 - protein, 236 aa, Bacillus licheniformis DSM 13
MENLLINGFGSSGGGAYQTVELNGKGTINGDIDCERFSCSGTGTVNGDVKADELRISGHAKLNGAIEASKVRIDGSAKVDGDVEAEKLKILGYAGISGHVKGDEAIVRGKTSIGKDCQVEHFSAEGYFTIDGLLNAEQIEIKVHGGESKVKEIGCRRIVCTAHQSKLFSFLSELISPPRLSAELIEGDHIELANTTAKVVRGNSVIIGENCDIGLIEYKEDIRVAETAKVGEKRKL

SEQ ID NO: 75 - DNA, 750 bp, Bacillus licheniformis DSM 13
  gene (1)..(750): yvmC; similar to proteins; BLi03566
  CDS (1)..(750)
atgacagagcttataatggagagcaaacaccagctattcaaaaccgaaactcttacccaaaactgcaatgaaatattaaaacgcagacgccatgttctcgtcggcatcagcccgtttaacagccgattttccgaagattatattcatcggcttatcgcctgggccgtccgtgagtttcagagtgtatccgtgcttttggcgggaaaggaagctgccaaccttctcgaagcgctcggcaccccacatgggaaggccgaacggaaagtcaggaaagaagtctcgcggaaccggagattcgctgaaaaggcgttggaagcgcatggcggaaatcccgaggacatccatacattttccgatttcgcgaaccagaccgcataccggaatttgcggatggaagtcgaagctgcctttttcgaccagacgcattttcgcaatgcctgcctggagatgtcgcatgcggctatcctcggacgggcccggggcactcggatggatgtcgtggaagtcagcgcagacatgctggagctggctgttgaatacgtcatcgctgaacttccgtttttcatcgccgcccctgatattttaggcgtcgaagagacgcttcttgcttatcaccggccatggaagctcggcgaacagatctcccgtaatgaatttgccgtcaaaatgcggccgaatcaaggatatctcatggtttccgaagcggacgaaagggtggaatctaaaagcatgcaggaggaacgagtatga

SEQ ID NO: 76 - protein, 249 aa, Bacillus licheniformis DSM 13
MTELIMESKHQLFKTETLTQNCNEILKRRRHVLVGISPFNSRFSEDYIHRLIAWAVREFQSVSVLLAGKEAANLLEALGTPHGKAERKVRKEVSRNRRFAEKALEAHGGNPEDIHTFSDFANQTAYRNLRMEVEAAFFDQTHFRNACLEMSHAAILGRARGTRMDVVEVSADMLELAVEYVIAELPFFIAAPDILGVEETLLAYHRPWKLGEQISRNEFAVKMRPNQGYLMVSEADERVESKSMQEERV

SEQ ID NO: 77 - DNA, 483 bp, Bacillus licheniformis DSM 13
  gene (1)..(483): yvnA; similar to proteins from B. subtilis; BLi03569
  CDS (1)..(483)
atgatgacagtggaaaacaagacgtatgagcaggcatttcaagccgttcaggattttattctcaaaagagaaaagcgggctcagcatgaacagcaggcgttcgcagaggaagcctttcaagaagaagaaagcatgcgtaagcattggaccctgacccagcttcacattatctcgcttatcagcgaaagcgaagcagacgtcaacaacgctttcctggcagcaaaactccaaatttcaaaagcggcagtgacaaaagcggtcaatgtgctcacaaagcacggaatgatcgaatcgcacaaaaaaccgaacaacaacaaagaattgtattacacattaaccgatgaggggaaaaaactggccgacattcatgacaggatgcatgaaattgcaaaacagcgctacatcgagctgtttgatcggttttccgaatccgagctgcaaaccgtcatccgctttttaaacgaatggtcaaaacacatttaa

SEQ ID NO: 78 - protein, 160 aa, Bacillus licheniformis DSM 13
MMTVENKTYEQAFQAVQDFILKREKRAQHEQQAFAEEAFQEEESMRKHWTLTQLHIISLISESEADVNNAFLAAKLQISKAAVTKAVNVLTKHGMIESHKKPNNNKELYYTLTDEGKKLADIHDRMHEIAKQRYIELFDRFSESELQTVIRFLNEWSKHI

SEQ ID NO: 79 - DNA, 630 bp, Bacillus licheniformis DSM 13
  gene (1)..(630): spoIIIAF; mutants block sporulation after engulfment; BLi02609
  CDS (1)..(630)
atggaatttctgacagagtggctcacgaatattattctatttattctgatggcgatcgtcatcgatatgcttctgccgaattcgagcatgcaaaaatacgcgaaaatggtgatcagcctgctcttgatcgttgtaatactgaacccgatcttctctttattcaggacagatccggatgtgatttttgagaagcttacaaaaaacggacaagttcagtcaaacgaaataaaaaatcagctgaattcagaaaaaaaagaaatacaagcctcacaacaagcatatatcttagaacagatggctgttcaattggaaaagaacgcagagggcaggtttacaagcgacaaatacaagatagaccgagtcgaggtctcttctgacagccagctgaaaacagagaaagacctcagtaagcatgcggaagtctcggtattcttgaaaccagcatcggaaaaaacggtccaagccgtcgctcctgtcgagatcaatacggaccggagctaccagtccatgcaggaaagagaaaagaaagagacgggggaagtcagagaacagctagcaggcatctgggaaataagccccgacaagattacagttcatatcgaagggggagaacgaagcggcaatgaataa

SEQ ID NO: 80 - protein, 209 aa, Bacillus licheniformis DSM 13
MEFLTEWLTNIILFILMAIVIDMLLPNSSMQKYAKMVISLLLIVVILNPIFSLFRTDPDVIFEKLTKNGQVQSNEIKNQLNSEKKEIQASQQAYILEQMAVQLEKNAEGRFTSDKYKIDRVEVSSDSQLKTEKDLSKHAEVSVFLKPASEKTVQAVAPVEINTDRSYQSMQEREKKETGEVREQLAGIWEISPDKITVHIEGGERSGNE

SEQ ID NO: 81 - DNA, 696 bp, Bacillus licheniformis DSM 13
  gene (1)..(696): spoIIIAG; mutants block sporulation after engulfment; BLi02608
  CDS (1)..(696)
atgaataaaagaacctggatcgaaaagctgatcggccacctgctcccgaaagacgagaaagacggaaaaaagctgacgaaatatcactactttctgctgctttttgttctcggcgtttccttcatgctcgtcagccagatcttctcttccgaaccttcccaagagccagcggcagatcagccggcggcttcacaaaaagctacgtctgaaagcaccgtacagagcggtgaaggagaaaaagaagtgttcaagcccgcctcagatgacaaaccgaaggaatcgatccaagattacgaacaggaatatgaaaatcagctcaaagacatattggaaaccatcatcggcgttgaggacgtgtcaatcgtcgtcaatgttgatgcaacctcattgaaaatattcgagaaaaacagaaaaacccaggaaacttcaacgaatgagacagataaacagggaggcaagcggacggtgtctgaaatgtcttcagacgaagaaatcgtcatcatcaaaaacggagataaagagacgcctgtcgtcgttcagacgaaaaagcccgatatcaggggtgttctcgttgtcgctcagggagtcgacaacgttcaaataaaaaagaccattattgaagcagtgacaagggttcttgatgttccgagccaccgcgtcgctgttgcccctaaaaaaatgaaggaggattcataa

SEQ ID NO: 82 - protein, 231 aa, Bacillus licheniformis DSM 13
MNKRTWIEKLIGHLLPKDEKDGKKLTKYHYFLLLFVLGVSFMLVSQIFSSEPSQEPAADQPAASQKATSESTVQSGEGEKEVFKPASDDKPKESIQDYEQEYENQLKDILETIIGVEDVSIVVNVDATSLKIFEKNRKTQETSTNETDKQGGKRTVSEMSSDEEIVIIKNGDKETPVVVQTKKPDIRGVLVVAQGVDNVQIKKTIIEAVTRVLDVPSHRVAVAPKKMKEDS

SEQ ID NO: 83 - DNA, 639 bp, Bacillus licheniformis DSM 13
  gene (1)..(639): spoIIIAH; mutants block sporulation after engulfment; BLi02607
  CDS (1)..(639)
atgatgctgaaaaaacaaacggtttggcttttaaccatgttaagtctcgtcgttgtactgagtgtctactacattatgtcgcccgaaggagaaaatgtcgtcacggttgatgacaaggaacaagttgccgctgaaaaagaaaaaccgatgaaagaagagcctgccaaggatggcaaagatgataccgcgcctgctaaagacaaaactaaagggaaagatacaaaagataaagaaacgtctgcgagtgagcagaacggagaggttgtcacagaggaatcatcgggtgatgaagatttattcacaacataccgcatggaaatggacgatcagcgcagcagggagagggaggaattaaccgaaatcgtcagaagcgataaagcgacggcaaaagaaaaaagcgaagcttacgacaagatgacagagctcagcgaagctgaaggaacggaaaagacccttgaaaccctcatcaaaacaaaaggctataaagacgccttggtcaacgccgacggcgataaagtcaatattacggtgaaggcgaaggagcactcgaaagccgctgccaccgatatcatcgacatcgtcgcaaaagaagtgaaaaacttaaacgatgtggctgtaacgttcgagccagcagattaa

SEQ ID NO: 84 - protein, 212 aa, Bacillus licheniformis DSM 13
MMLKKQTVWLLTMLSLVVVLSVYYIMSPEGENVVTVDDKEQVAAEKEKPMKEEPAKDGKDDTAPAKDKTKGKDTKDKETSASEQNGEVVTEESSGDEDLFTTYRMEMDDQRSREREELTEIVRSDKATAKEKSEAYDKMTELSEAEGTEKTLETLIKTKGYKDALVNADGDKVNITVKAKEHSKAAATDIIDIVAKEVKNLNDVAVTFEPAD

SEQ ID NO: 85 - DNA, 888 bp, Bacillus licheniformis DSM 13
  gene (1)..(888): pstA; phosphate ABC transporter (permease); BLi02674
  CDS (1)..(888)
atgaacagcaaagtaacagatcgtttagcaaccggtgtttttggattgattgctgcggtaatcgccgctattcttgtcggtttgttcgcttatatcatcatcaatggtgtttcgcatatcagcctcagctttttaacttcgcgttcaagtgcgctgtcaagcggcggaggaattcgagatcagctgttcaactcgttttatattctcgtgctgacgatgctgatcacggttcccctgggagtcggcgggggaatttatatggcggaatatgcgcccgcaaacaaattgaccgattttatccgtgtctgtattgaagttttgtcatcccttccttcgatcgttatggggatgttcggcatgctgatgtttgtaaatctgaccggatgggggtacagcattatcggaggagcgcttgctttgacgattttcaacctgcctgtcatggtcagggtgacagaagatgcgatccgctccgtacctaaggagcaaaaagaggcttcccttgctttaggcgtgtccaaatggcacacgattaaaacggtattgattcctggcgcggtgccgtccatcatcacaggcgcgattcttgcgtcgggccgtgtatttggcgaagctgccgctttgctgtttacagccggactaaccacgccgagactgaattttgccgattggaatccgttttctgagacatcgccgctcaatattttcagaccggctgaaacgctggccgttcatatttggaatgtaaacacacaggggatcatccccgatgctgaagcgatcgcaaacggcgcatccgccgttctcgttctgtccgtcttgatctttaacctggcagcccgatggctcggctcttttattcataaaaagttaacggcaaacaaataa

SEQ ID NO: 86 - protein, 295 aa, Bacillus licheniformis DSM 13
MNSKVTDRLATGVFGLIAAVIAAILVGLFAYIIINGVSHISLSFLTSRSSALSSGGGIRDQLFNSFYILVLTMLITVPLGVGGGIYMAEYAPANKLTDFIRVCIEVLSSLPSIVMGMFGMLMFVNLTGWGYSIIGGALALTIFNLPVMVRVTEDAIRSVPKEQKEASLALGVSKWHTIKTVLIPGAVPSIITGAILASGRVFGEAAALLFTAGLTTPRLNFADWNPFSETSPLNIFRPAETLAVHIWNVNTQGIIPDAEAIANGASAVLVLSVLIFNLAARWLGSFIHKKLTANK

SEQ ID NO: 87 - DNA, 828 bp, Bacillus licheniformis DSM 13
  gene (1)..(828): pstBA; phosphate ABC transporter; BLi02673
  CDS (1)..(828)
atggctcaggttcagatgccggttttggaaaccgtgcgcgacaatatcggaatcgctccgttccctgaagcgaaagagcatattttagaagtgaaaaatttaaatatttactatggagaaaagcgggccgtcaacgacatttccatgcgtattgacaaacatgcggtcacggctttgatcggtccgtcaggctgcggaaaatcgacttttttaagaagcatcaaccggatgaacgatctgattccatcagccagaacatccggagaaatacggtatgaagggcttaacatccttgattccaatatcaatgtcgtcaatctgagaagggagatcggcatggtatttcagaagccgaacccctttccaaaatcgatttacagcaacattacccacgcattaaaatatgcgggcgaacggcggaaatccgtgcttgatgagatcgtggaagaaagcttgacgaaagccgcgctgtgggatgaagtgaaagacaggctgcatgagtcggctctttcgctgtcaggcgggcagcagcagcgtctctgcatcgcccggacgctcgcgatgaagcccgaagtcctgctcttggatgagcctgcttcagcgctcgacccgatttcaaacgcgaaaatagaggaattaatcgtcggtttaaaagaggaatactcgatcatcattgtcacgcataacatgcagcaagcttcaagaatttccgatcaaacggcgtttttccttaacggagatttaatcgaatacgatcgtacggaaaagatttttatgaatccttcagaaaaaaagacagaggattacatcaacggcagatttggataa

SEQ ID NO: 88 - protein, 275 aa, Bacillus licheniformis DSM 13
MAQVQMPVLETVRDNIGIAPFPEAKEHILEVKNLNIYYGEKRAVNDISMRIDKHAVTALIGPSGCGKSTFLRSINRMNDLIPSARTSGEIRYEGLNILDSNINVVNLRREIGMVFQKPNPFPKSIYSNITHALKYAGERRKSVLDEIVEESLTKAALWDEVKDRLHESALSLSGGQQQRLCIARTLAMKPEVLLLDEPASALDPISNAKIEELIVGLKEEYSIIIVTHNMQQASRISDQTAFFLNGDLIEYDRTEKIFMNPSEKKTEDYINGRFG

SEQ ID NO: 89 - DNA, 780 bp, Bacillus licheniformis DSM 13
  gene (1)..(780): pstBB; phosphate ABC transporter; BLi02672
  CDS (1)..(780)
atgagcgctgtaacagctgtaaagccagaagatgtataccagatcaacgacttgaatttatggtacggcgaacatcacgcattaaagaacattaacttgacaatacccgagtacgaaattaccgccatcattggaccgtcagggtgcggaaagtcgacattcattaaaacgctgaatctcatgatcaatatggttccgaatgtcaaaatgaccggagagatcagctacaacggaacgaacgtactcaacagcaaagtcgaccttgtcgagttacggaaaaaagtcggcatggtgtttcaaaaaggaaacccgtttccccagtcgatctttgataatgtcgcatacggaccaagaatccatggattgaaaaacaaaaaagaactgaatgagagagtggaaaaggcgcttgtggatgtagcgctgtgggatgaagtgaaagaccgtcttcattcccaggcgctcggactttccggggggcagcagcagcgcctctgcatcgcaagggcgctggccaccaatcctgacgttctcttgatggatgaaccgacatccgcccttgatcccgtttcaactttgaaaatcgaagagctgatgctcaagctgaaagagaaatatacaattgccatcgtcacgcacaacatgcagcaggcctcgagaatttccgatcaaacggcgtttttcctgatgggcgagctgattgagtgcaatgatacggtcaaaatgttttcaagtcctgacgatcatcggacgaaagattatataacaggccggttcggatga

SEQ ID NO: 90 - protein, 259 aa, Bacillus licheniformis DSM 13
MSAVTAVKPEDVYQINDLNLWYGEHHALKNINLTIPEYEITAIIGPSGCGKSTFIKTLNLMINMVPNVKMTGEISYNGTNVLNSKVDLVELRKKVGMVFQKGNPFPQSIFDNVAYGPRIHGLKNKKELNERVEKALVDVALWDEVKDRLHSQALGLSGGQQQRLCIARALATNPDVLLMDEPTSALDPVSTLKIEELMLKLKEKYTIAIVTHNMQQASRISDQTAFFLMGELIECNDTVKMFSSPDDHRTKDYITGRFG

SEQ ID NO: 91 - DNA, 930 bp, Bacillus licheniformis DSM 13
  gene (1)..(930): pstC; phosphate ABC transporter (permease); BLi02675
  CDS (1)..(930)
atggttcaaaaaatagagaaactgtccgcgaaagagcggctgatcagctccaagcataatcgcaaaatggatgaaatccgcgggaggattttggttgcgtcgtgcgcctttatcatgatggcggcagcgatttcgatcacaatttttctcggaaataaggggttgcaatcttttctggccaatggtgtaagtccaattgagttcttaaccagtctagattggaatccaacgaacgaaaaccctgaatacggagcacttccgtttatactcggatcttttgcagtgacccttcttgcatctttgattgctgcaccgctaggaataggaggtgcgattttcatgacggaaatcgcgccatcatggggcagaaaagtgctgcagccggttgtcgaattgcttgttggcattccttccgtcgtttatggattcataggccttacagtgttagtgccgttcatttcacagtttaagtcaagcggatcgggccacagccttctggcgggaacgatcgtcctggctgtcatgattttgccgacggtcacttcgatttcaacggatgcgctcatgtcgctgccgaaaaatctccgcgaaggatcatacgcattaggcgcaacccgctggcagacgatccgcaaagtgctgataccggcagcgctgccttcgcttctgacagccgtcgtcttgggaatggcgagggcgttcggcgaagcgctggccgtgcagatggtcatcggaaattcccgaaatctgtttgagagccttttagaccctgcggggacattgacaacgatcattacgttgaacatgggccatacaacatacgggagcgtggaaaacaatacgctctggtcaatggggcttgtccttctgatcatgtcgttcttgttcatcctgctcatccgctatctgtcatccaggaggaaactgtaa

SEQ ID NO: 92 - protein, 309 aa, Bacillus licheniformis DSM 13
MVQKIEKLSAKERLISSKHNRKMDEIRGRILVASCAFIMMAAAISITIFLGNKGLQSFLANGVSPIEFLTSLDWNPTNENPEYGALPFILGSFAVTLLASLIAAPLGIGGAIFMTEIAPSWGRKVLQPVVELLVGIPSVVYGFIGLTVLVPFISQFKSSGSGHSLLAGTIVLAVMILPTVTSISTDALMSLPKNLREGSYALGATRWQTIRKVLIPAALPSLLTAVVLGMARAFGEALAVQMVIGNSRNLFESLLDPAGTLTTIITLNMGHTTYGSVENNTLWSMGLVLLIMSFLFILLIRYLSSRRKL

SEQ ID NO: 93 - DNA, 456 bp, Bacillus licheniformis DSM 13
  gene (1)..(456): putative ribonuclease; BLi03719
  misc_feature (1)..(3): First codon translated as Met.
  CDS (1)..(456)
gtgaaaaaaatattatcaactcttgcgcttgggtttgttctcgcgctgggctttctggcggggaacctttttacaagctcagctgaagggccgcaggcagaaaaaggcggcaccgttcagtcaaatgaagtcatcaatacatttgaaggtgttgccgactatattgtgaaatacggcaggctgcctgacaacttcatcacaaaagccgaggcgtcaaaattaggctgggatccgcaaaaaggcaatctggcagaagtagcgcctggaaaaagcatcggcggcgatatctttcaaaacagggaaaagctgcttccagatgcatccggacgcgtatggagagaagccgacatcaactatacatcgggattccgggggtcagaccggatcgtctattctaacgacagactggtttataaaacgactgaccactataaaacgtttacgagaatgagatga

SEQ ID NO: 94 - protein, 151 aa, Bacillus licheniformis DSM 13
  misc_feature (1)..(3): First codon translated as Met.
VKKILSTLALGFVLALGFLAGNLFTSSAEGPQAEKGGTVQSNEVINTFEGVADYIVKYGRLPDNFITKAEASKLGWDPQKGNLAEVAPGKSIGGDIFQNREKLLPDASGRVWREADINYTSGFRGSDRIVYSNDRLVYKTTDHYKTFTRMR